(19) United States
(12) Patent Application Publication
(10) Pub. No.: US 2008/0032299 A1
(43) Pub. Date: Feb. 7, 2008

(54) Methods for prognosis and treatment of solid tumors

(75) Inventors: Michael E. Burczynski, Swampscott, MA (US); Natalie C. Twine, Goffstown, NH (US); William L. Trepicchio, Andover, MA (US); Andrew Strahs, Maynard, MA (US); Fred Immermann, Suffern, NY (US); Donna K. Slonim, North Andover, MA (US); Andrew J. Dorner, Lexington, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/715,606

(22) Filed: Mar. 8, 2007

Related U.S. Application Data
(63) Continuation of application No. 10/834,268, filed on Apr. 29, 2004, now abandoned.
(60) Provisional application No. 60/466,067, filed on Apr. 29, 2003.
(60) Provisional application No. 60/538,246, filed on Jan. 23, 2004.

(51) Int. Cl. C12Q 1/68; C40B 40/08; C40B 40/10; G06G 7/48

(52) U.S. Cl. 435/6; 506/17; 506/18; 703/11

(57) ABSTRACT

Solid tumor prognosis genes, and methods, systems and equipment of using these genes for the prognosis and treatment of solid tumors. Prognosis genes for a solid tumor can be identified by the present invention. The expression profiles of these genes in peripheral blood mononuclear cells (PBMCs) are correlated with clinical outcome of the solid tumor. The prognosis genes of the present invention can be used as surrogate markers for predicting clinical outcome of a solid tumor in a patient of interest. These genes can also be used to select a treatment which has a favorable prognosis for the solid tumor of the patient of interest.

### CROSS-REFERENCE TO RELATED APPLICATIONS

The present application is a continuation of U.S. application Ser. No. 10/834,268, filed on Apr. 29, 2004, which claims the benefit of U.S. Provisional Application No. 60/466,067, filed Apr. 29, 2003, and U.S. Provisional Application No. 60/538,246, filed Jan. 23, 2004, the disclosures of each of which are hereby incorporated by reference in their entireties.

### REFERENCE TO SEQUENCE LISTING AND TABLES ON COMPACT DISC

This application includes as part of the originally filed subject-matter two compact discs, labeled “Copy 1 CD Containing Sequence Listing and Large Tables” and “Copy 2 CD Containing Sequence Listing and Large Tables.” Each compact disc includes the following files: “Sequence Listing.ST25.txt” (5,454 KB, created Mar. 8, 2007), “Table 3—Spearman Correlation of Baseline Expression with Clinical Outcome.txt” (298 KB, created Jan. 19, 2004), “Table 4—Qualifiers and the Corresponding Entrez and Unigene Accession Nos.txt” (179 KB, created Jan. 20, 2004), “Table 5—Genes and Gene Titles.txt” (331 KB, created Jan. 21, 2004), and “Table 8—Cox Regression of Clinical Outcome on Baseline Gene Expression.txt” (294 KB, created Jan. 19, 2004). The contents of the compact discs labeled “Copy 1 CD Containing Sequence Listing and Large Tables” and “Copy 2 CD Containing Sequence Listing and Large Tables” are hereby incorporated by reference herein in their entireties.

### TECHNICAL FIELD

The present invention relates to solid tumor prognosis genes and methods of using these genes for the prognosis or treatment of solid tumors.

### BACKGROUND

Expression profiling studies in primary tissues have demonstrated that there exist transcriptional differences between normal and malignant tissues. See, for example, Su, et al., CANCER RES, 61:7388-7393 (2001); and Ramaswamy, et al., PROC NATL ACAD SCI U.S.A., 98:15149-15151 (2001). Recent clinical analyses have also identified expression profiles within tumors that appear to be highly correlated with certain measures of clinical outcomes. One study has demonstrated that expression profiling of primary tumor biopsies yields prognostic “signatures” that rival or may even out-perform currently accepted standard measures of risk in cancer patients. See van de Vijver, et al., N ENGL J MED, 347:1999-2009 (2002).

### SUMMARY OF THE INVENTION

The present invention provides methods, systems and equipment for prognosis or selection of treatment of solid tumors. Prognosis genes for a solid tumor can be identified by the present invention. The expression profiles of these genes in peripheral blood mononuclear cells (PBMCs) are correlated with clinical outcome of the solid tumor. These genes can be used as surrogate markers for predicting clinical outcome of the solid tumor in a patient of interest. These genes can also be used to identify or select treatments which have favorable prognoses for the patient of interest.

In one aspect, the present invention provides methods that are useful for the prognosis or selection of treatment of a solid tumor in a patient of interest. The methods include comparing an expression profile of one or more prognosis genes in a peripheral blood sample of the patient of interest to at least one reference expression profile of the prognosis genes. Each of the prognosis genes is differentially expressed in PBMCs of a first class of patients as compared to PBMCs of a second class of patients. Both classes of patients have a solid tumor, and each class of patients has a different clinical outcome. In many embodiments, the prognosis genes are substantially correlated with a class distinction between the two classes of patients.

Solid tumors amenable to the present invention include, but are not limited to, renal cell carcinoma (RCC), prostate cancer, head/neck cancer, and other tumors that do not have their origin in blood or lymph cells.

Clinical outcome can be measured by any clinical indicator. In one embodiment, clinical outcome is determined based on clinical classifications such as complete response, partial response, minor response, stable disease, progressive disease, non-progressive disease, or any combination thereof. In another embodiment, clinical outcome is measured by time to disease progression (TTP) or time to death (TTD). In still another embodiment, clinical outcome is prognosticated by using traditional risk assessment methods, such as Motzer risk classification for RCC. Other patient responses to a therapeutic treatment can also be used to measure clinical outcome. Examples of solid tumor treatments include, but are not limited to, drug therapy (e.g., CCI-779 therapy), chemotherapy, hormone therapy, radiotherapy, immunotherapy, surgery, gene therapy, anti-angiogenesis therapy, palliative therapy, or any combination thereof.

In many embodiments, the reference expression profile(s) includes an average expression profile of the prognosis genes in peripheral blood samples of reference patients. In many instances, the reference patients have the same solid tumor as the patient of interest, and the clinical outcome of the reference patients are either known or determinable.

The peripheral blood samples of the patient of interest and reference patients can be whole blood samples, or blood samples comprising enriched or purified PBMCs. Other types of blood samples can also be employed in the present invention. In one embodiment, all of the peripheral blood samples are baseline samples which are isolated from respective patients prior to a therapeutic treatment of the patients.

Any comparison method can be used to compare the expression profile of the patient of interest to the reference expression profile(s). In one embodiment, the comparison is based on the absolute or relative peripheral blood expression level of each prognosis gene. In another embodiment, the comparison is based on the ratios between expression levels of two or more prognosis genes. In yet another embodiment, the reference expression profiles include at least two distinct expression profiles, each being derived from a different class of reference patients. The comparison of the expression profile of the patient of interest to the reference expression profiles can be carried out by using methods including, but not limited to, hierarchical clustering, k-nearest-neighbors, or weighted-voting algorithm.

In still another embodiment, the methods of the present invention include selecting a treatment which has a favorable prognosis for the solid tumor in the patient of interest.

In another aspect, the present invention provides other methods useful for the prognosis or selection of treatment of a solid tumor in a patient of interest. These methods include comparing an expression profile of one or more prognosis genes in a peripheral blood sample of the patient of interest to at least one reference expression profile of the prognosis genes, where each of the prognosis genes is differentially expressed in PBMCs of a first class of patients as compared to PBMCs of a second class of patients. Each of the first and second classes is a subcluster formed by an unsupervised clustering analysis of gene expression profiles in PBMCs of patients who have the solid tumor. In one embodiment, the majority of the first class of patients has a first clinical outcome, and the majority of the second class of patients has a second clinical outcome.

In yet another aspect, the present invention further provides methods useful for the prognosis or selection of treatment of a solid tumor in a patient of interest. The methods include comparing an expression profile of one or more prognosis genes in a peripheral blood sample of the patient of interest to at least one reference expression profile of the prognosis genes, where the expression levels of each of the prognosis genes in PBMCs of patients having the solid tumor are correlated with clinical outcomes of these patients. The association between PBMC expression levels and clinical outcome can be determined by a statistical method (e.g., Spearman's rank correlation or Cox proportional hazard regression model) or a class-based correlation metric (e.g., neighborhood analysis). In one embodiment, the solid tumor is RCC, and clinical outcome is measured by patient response to a CCI-779 therapy. In another embodiment, the prognosis genes include at least one gene selected from Tables 6a, 6b, 6c, 6d, 9a, 9b, 9c, 9d, 10, 11, 12, 13, 16, 20, and 21.

The present invention also features systems useful for the prognosis or selection of treatment of a solid tumor in a patient of interest. The systems include (1) a memory or a storage medium comprising data that represent an expression profile of one or more prognosis genes in a peripheral blood sample of the patient of interest, (2) a storage medium comprising data that represent at least one reference expression profile of the prognosis genes, (3) a program capable of comparing the expression profile of the patient of interest to the reference expression profile, and (4) a processor capable of executing the program. The expression levels of the prognosis genes in PBMCs of patients having the solid tumor are correlated with clinical outcomes of the patients.

Moreover, the present invention features nucleic acid or protein arrays useful for the prognosis or selection of treatment of a solid tumor in a patient of interest. The nucleic acid or protein arrays include concentrated probes for solid tumor prognosis genes.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The drawings are provided for illustration, not limitation.

FIG. 1A depicts expression profiles of class-correlated genes identified by nearest-neighbor analysis of patients with survival of less than 150 days versus patients with survival of greater than 550 days. The relative expression levels of the class-correlated genes (rows) are indicated for each patient (columns) according to the normalized expression level scale.

FIG. 1B shows the comparison of the signal to noise (S2N) similarity metric scores for class-correlated genes identified in FIG. 1A relative to S2N scores for the top 1%, 5%, and 50% of scores for class-correlated genes resulting from randomly permuted data sets.

FIG. 1C illustrates training set cross validation results for predictor gene sets of increasing size. Each predictor set was evaluated by cross validation to identify the predictor set with the highest accuracy for classification of the samples. In these analyses, a 58 gene predictor set (77% accuracy) was the optimal classifier.

FIG. 1D demonstrates cross validation results for each sample using the 58-gene predictor identified in FIG. 1C. A leave-one-out cross validation was performed and the prediction strengths were calculated for each sample in the analysis. For the purposes of illustration, confidence scores accompanying calls of “TTD>550 days” were assigned positive values, while prediction strengths accompanying calls of “TTD<150 days” were assigned negative values.

FIG. 2A shows the relative gene expression levels of a 42-gene classifier for the comparison of patients with intermediate versus poor Motzer risk classification.

FIG. 2B shows the relative gene expression levels for an 18-gene classifier identified in the comparison of patients with progressive disease versus any other clinical response.

FIG. 2C demonstrates the relative gene expression levels for a 6-gene classifier identified in the comparison of patients in the lower versus upper quartiles of time to disease progression.

FIG. 2D shows the relative gene expression levels for a 52-gene classifier identified in the comparison of patients in the lower versus upper quartiles of survival/time to death.

FIG. 2E depicts the relative expression levels for a 12-gene classifier identified in the comparison of patients with early (time to disease progression<106 days) versus all other times to disease progression (TTP≧106 days).

FIG. 3A illustrates the dendrogram of an unsupervised hierarchical clustering of baseline PBMC profiles in 45 RCC patients using all expressed genes present in at least one sample and possessing a frequency of greater than 10 ppm in at least one sample (5,424 genes total). PBMC expression profiles in the poor prognosis cluster are indicated by subcluster “A,” where 9 out of 12 patients with PBMC profiles in this subcluster exhibited survival of less than a year. PBMC expression profiles in the good prognosis cluster are indicated by subcluster “C,” where 10 out of 12 patients with PBMC profiles in this subcluster exhibited survival of greater than a year. The median survival for patients in subclusters A, B, C, and D is 281 days, 566 days, 573 days, and 502 days, respectively.

FIG. 3B shows baseline expression profiles of selected genes in RCC patients. The dendrogram of sample relatedness is indicated.

FIG. 4A illustrates the Kaplan-Meier survival curve for patients in the poor and good prognosis subclusters segregated on the basis of gene expression pattern.

FIG. 4B illustrates the Kaplan-Meier survival curve for patients in the poor and good prognosis subclusters segregated on the basis of Motzer risk assessment.

FIG. 5A demonstrates the result of supervised identification of a gene classifier for assigning class membership to patients in the good and poor prognosis subclusters. The relative expression levels of the most class-correlated gene (rows) are indicated for each patient (columns) according to the scale described in FIG. 1A.

FIG. 5B shows cross validation results for each sample using the gene classifier of FIG. 5A. A leave-one-out cross validation was performed and the confidence scores were calculated for each sample in the analysis. Similar to FIG. 1D, for the purposes of illustration, prediction strengths accompanying calls of “survival>1 year” were assigned positive values, while prediction strengths accompanying calls of “survival<1 year” were assigned negative values. Asterisks identify the false positives in this clinical assay designed to identify short survival times, and arrowheads indicate false negatives.

FIG. 6A shows the optimal gene classifier for year-long survival identified by nearest-neighbor analysis using a more stringent filter (at least 25% present calls, and an average frequency no less than 5 ppm). A GeneCluster gene selection approach identifies genes distinguishing patients with survival less than 365 days versus patients with survival greater than 365 days in the training set. The relative expression levels of the most class-correlated genes (rows) are indicated for each of the patients in the training set (columns) according to the scale described in FIG. 1A.

FIG. 6B evaluates prediction accuracy of gene classifiers of increasing size. Accuracy of class assignment for gene classifiers containing between 2 and 60 genes in steps of 2, and 60-200 genes in steps of 10, were evaluated by leave-one-out cross validation on the training set of samples. The smallest predictive model with the highest accuracy was selected (20 gene predictor, indicated by the arrow).

FIG. 6C demonstrates the result of evaluation of the optimal predictive model of FIG. 6B on an untested set of RCC PBMC profiles. A k-nearest-neighbors algorithm using the 20 gene classifier was used to assign class membership to the remaining 14 PBMC profiles, and the prediction strengths associated with the class assignments are presented for each sample in the analysis. For the purposes of illustration, confidence scores accompanying calls of “TTD<365 days” were assigned positive values, while confidence scores accompanying calls of “TTD>365 days” were assigned negative values. The overall accuracy of the gene classifier was 72%. By defining the clinical assay as the identification of favorable outcome, eight of eight patients with favorable outcome were correctly identified as having survival greater than one year (positive predictive value of 100%).

FIG. 7A illustrates the optimal gene classifier for greater than 106 day time to progression identified by nearest-neighbor analysis using a more stringent filter (at least 25% present calls, and an average frequency no less than 5 ppm). A GeneCluster gene selection approach identifies genes distinguishing patients with TTP less than 106 days versus patients with TTP greater than 106 days in the training set. The relative expression levels of the most class-correlated genes (rows) are indicated for each of the patients in the training set (columns) according to the scale of FIG. 1A.

FIG. 7B indicates prediction accuracy of gene classifiers of increasing size. Accuracy of class assignment for gene classifiers containing between 2 and 60 genes in steps of 2, and 60-200 genes in steps of 10, were evaluated by leave-one-out cross validation on the training set of samples. The smallest predictive model with the highest accuracy was selected (30 gene predictor, indicated by the arrow).

FIG. 7C shows the result of evaluation of the optimal predictive model of FIG. 7B on an untested set of RCC PBMC profiles. A k-nearest-neighbors algorithm using the 30 gene classifier was used to assign class membership to the remaining 14 PBMC profiles, and the prediction strengths associated with the class assignments are presented for each sample in the analysis. For the purposes of illustration, confidence scores accompanying calls of “TTP<106 days” were assigned positive values, while confidence scores accompanying calls of “TTD>106 days” were assigned negative values. The overall accuracy of the gene classifier was 85%. By defining the clinical assay as the identification of favorable outcome, eight of ten patients with favorable outcome were correctly identified as having TTP greater than one 106 days (positive predictive value of 80%) and three of three patients with poor outcome were correctly predicted to have TTP less than 106 days (negative predictive value 100%).

### DETAILED DESCRIPTION

The present invention provides methods that are useful for prognosis or selection of treatment of solid tumors. These methods employ prognosis genes that are differentially expressed in peripheral blood samples of solid tumor patients who have different clinical outcomes. In many embodiments, the peripheral blood expression profiles of these prognosis genes are correlated with patients' clinical outcome or prognosis under a statistical method or a correlation model. In many other embodiments, solid tumor patients can be divided into at least two classes based on patients' clinical outcome or prognosis, and the prognosis genes are substantially correlated with a class distinction between these two classes of patients under a neighborhood analysis.

The prognosis genes of the present invention can be used as surrogate markers for the prediction of clinical outcome of solid tumors. The prognosis genes of the present invention can also be used for the identification of optimal treatments of solid tumors. Different patients may have distinct clinical responses to a therapeutic treatment due to individual heterogeneity of the molecular mechanism of the disease. The identification of gene expression patterns that correlate with patient response allows clinicians to select treatments based on predicted patient responses and thereby avoid adverse reactions. This provides improved power and safety of clinical trials and increased benefit/risk ratio for drugs and other therapeutic treatments. Peripheral blood is a tissue that can be routinely obtained from patients in a minimally invasive manner. By determining the correlation between patient outcome and gene expression profiles in peripheral blood samples, the present invention represents a significant advance in clinical pharmacogenomics and solid tumor treatment.

Various aspects of the invention are described in further detail in the following subsections. The use of subsections is not meant to limit the invention. Each subsection may apply to any aspect of the invention. In this application, the use of “or” means “and/or” unless stated otherwise.

I. General Methods for Identifying Solid Tumor Prognosis Genes

Previous studies demonstrated that baseline expression profiles in PBMCs from solid tumor patients were significantly distinct from those of disease-free subjects. See U.S. Provisional Application Ser. No. 60/459,782, filed Apr. 3, 2003, U.S. Provisional Application Ser. No. 60/427,982, filed Nov. 21, 2002, and U.S. patent application Ser. No. 10/717,597, filed Nov. 21, 2003, all of which are incorporated herein by reference. Studies also showed that gene expression profiles in PBMCs were predictive of anti-cancer drug activity in vivo. See U.S. Provisional Application Ser. No. 60/446,133, filed Feb. 11, 2003, and U.S. patent application Ser. No. 10/775,169, filed Feb. 11, 2004, both of which are incorporated herein by reference. In addition, studies indicated that PBMC baseline expression profiles were correlated with clinical outcomes of RCC or other non-blood diseases. See U.S. Provisional Application Ser. No. 60/466,067, filed Apr. 29, 2003, which is incorporated herein by reference.

The present invention further evaluates the correlation between peripheral blood gene expression and clinical outcome of solid tumors. Prognosis genes for a variety of solid tumors can be identified by the present invention. These genes are differentially expressed in peripheral blood samples of solid tumor patients who have different clinical outcomes. In many embodiments, the peripheral blood expression profiles of the prognosis genes of the present invention are correlated with patient outcome under statistical methods or correlation models. Exemplary statistical methods and correlation models include, but are not limited to, Spearman's rank correlation, Cox proportional hazard regression model, ANOVA/t test, nearest-neighbor analysis, and other rank tests, survival models or class-based correlation metrics.

Solid tumors amenable to the present invention include, without limitation, RCC, prostate cancer, head/neck cancer, ovarian cancer, testicular cancer, brain tumor, breast cancer, lung cancer, colon cancer, pancreas cancer, stomach cancer, bladder cancer, skin cancer, cervical cancer, uterine cancer, and liver cancer. In one embodiment, the solid tumors do not have their origin in blood or lymph (hematopoetic) cells. Solid tumors can be measured or evaluated using direct or indirect visualization procedures. Suitable visualization methods include, but are not limited to, scans (such as X-rays, computerized axial tomography (CT), magnetic resonance imaging (MRI), positron emission tomography (PET), or ultrasonography (U/S)), biopsy, palpation, endoscopy, laparoscopy, and other suitable means as appreciated by those skilled in the art.

Clinical outcome of solid tumors can be assessed by numerous criteria. In many embodiments, clinical outcome is assessed based on patients' response to a therapeutic treatment. Examples of clinical outcome measures include, without limitation, complete response, partial response, minor response, stable disease, progressive disease, time to disease progression (TTP), time to death (TTD or Survival), or any combination thereof. Examples of solid tumor treatments include, without limitation, drug therapy (e.g., CCI-779 therapy), chemotherapy, hormone therapy, radiotherapy, immunotherapy, surgery, gene therapy, anti-angiogenesis therapy, palliative therapy, or any combination thereof, or other conventional or non-conventional therapies.

In one embodiment, clinical outcome is evaluated based on the WHO Reporting Criteria, such as those described in WHO Publication, No. 48 (World Health Organization, Geneva, Switzerland, 1979). Under the Criteria, uni- or bidimensionally measurable lesions are measured at each assessment. When multiple lesions are present in any organ, up to 6 representative lesions can be selected, if available.

In another embodiment, clinical outcome is determined based on a classification system composed of clinical categories such as complete response, partial response, minor response, stable disease, progressive disease, or any combination thereof. “Complete response” (CR) means complete disappearance of all measurable and evaluable disease, determined by two observations not less than 4 weeks apart. There is no new lesion and no disease related symptom. “Partial response” (PR) in reference to bidimensionally measurable disease means decrease by at least about 50% of the sum of the products of the largest perpendicular diameters of all measurable lesions as determined by 2 observations not less than 4 weeks apart. “Partial response” in reference to unidimensionally measurable disease means decrease by at least about 50% in the sum of the largest diameters of all lesions as determined by 2 observations not less than 4 weeks apart. It is not necessary for all lesions to have regressed to qualify for partial response, but no lesion should have progressed and no new lesion should appear. The assessment should be objective. “Minor response” in reference to bidimensionally measurable disease means about 25% or greater decrease but less than about 50% decrease in the sum of the products of the largest perpendicular diameters of all measurable lesions. “Minor response” in reference to unidimensionally measurable disease means decrease by at least about 25% but less than about 50% in the sum of the largest diameters of all lesions.

“Stable disease” (SD) in reference to bidimensionally measurable disease means less than about 25% decrease or less than about 25% increase in the sum of the products of the largest perpendicular diameters of all measurable lesions. “Stable disease” in reference to unidimensionally measurable disease means less than about 25% decrease or less than about 25% increase in the sum of the diameters of all lesions. No new lesions should appear. “Progressive disease” (PD) refers to a greater than or equal to about a 25% increase in the size of at least one bidimensionally (product of the largest perpendicular diameters) or unidimensionally measurable lesion or appearance of a new lesion. The occurrence of pleural effusion or ascites is also considered as progressive disease if this is substantiated by positive cytology. Pathological fracture or collapse of bone is not necessarily evidence of disease progression.

In yet another embodiment, overall subject tumor response for uni- and bidimensionally measurable disease is determined according to Table 1.

TABLE 1
Overall Subject Tumor Response

| Response in | Response in | |
|---|---|---|
| Bidimensionally | Unidimensionally | Overall Subject |
| Measurable Disease | Measurable Disease | Tumor Response |
| PD | Any | PD |
| Any | PD | PD |
| SD | SD or PR | SD |
| SD | CR | PR |
| PR | SD or PR or CR | PR |
| CR | SD or PR | PR |
| CR | CR | CR |

Overall subject tumor response for non-measurable disease can be assessed, for instance, in the following situations:
a) Overall complete response: if non-measurable disease is present, it should disappear completely. Otherwise, the subject cannot be considered as an “overall complete responder.”
b) Overall progression: in case of a significant increase in the size of non-measurable disease or the appearance of a new lesion, the overall response will be progression.

Clinical outcome can also be assessed by other criteria. For instance, clinical outcome can be measured by TTP or TTD. TTP refers to the interval from the date of initiation of a therapeutic treatment until the first day of measurement of progressive disease. TTD refers to the interval from the date of initiation of a therapeutic treatment to the time of death, or censored at the last date known alive.

Moreover, clinical outcome can include prognoses based on traditional clinical risk assessment methods. In many cases, these risk assessment methods employ numerous prognostic factors to classify patients into different prognosis or risk groups. One example is Motzer risk assessment for RCC, as described in Motzer, et al., J CLIN ONCOL, 17:2530-2540 (1999). Patients in different risk groups may have different responses to a therapy.

Peripheral blood samples employed in the present invention can be isolated from solid tumor patients at any disease or treatment stage. In one embodiment, the peripheral blood samples are isolated from solid tumor patients prior to a therapeutic treatment. These blood samples are “baseline samples” with respect to the therapeutic treatment.

A variety of peripheral blood samples can be used in the present invention. In one embodiment, the peripheral blood samples are whole blood samples. In another embodiment, the peripheral blood samples comprise enriched PBMCs. By “enriched,” it means that the percentage of PBMCs in the sample is higher than that in whole blood. In some cases, the PBMC percentage in an enriched sample is at least 1, 2, 3, 4, 5 or more times higher than that in whole blood. In some other cases, the PBMC percentage in an enriched sample is at least 90%, 95%, 98%, 99%, 99.5%, or more. Blood samples containing enriched PBMCs can be prepared using any method known in the art, such as Ficoll gradients centrifugation or CPTs (cell purification tubes).

The relationship between peripheral blood gene expression profiles and patient outcome can be evaluated using global gene expression analyses. Methods suitable for this purpose include, but are not limited to, nucleic acid arrays (such as cDNA or oligonucleotide arrays), 2-dimensional SDS-polyacrylamide gel electrophoresis/mass spectrometry, and other high throughput nucleotide or polypeptide detection techniques.

Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,288,220 and 6,391,562.

The polynucleotides to be hybridized to nucleic acid arrays can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides derived from one sample, such as PBMCs from a patient in a selected outcome class, are hybridized to the probes on a nucleic acid array. Signals detected after the formation of hybridization complexes correlate to the polynucleotide levels in the sample. In the differential hybridization format, polynucleotides derived from two biological samples, such as one from a patient in a first outcome class and the other from a patient in a second outcome class, are labeled with different labeling moieties. A mixture of these differently labeled polynucleotides is added to a nucleic acid array. The nucleic acid array is then examined under conditions in which the emissions from the two different labels are individually detectable. In one embodiment, the fluorophores Cy3 and Cy5 (Amersham Pharmacia Biotech, Piscataway N.J.) are used as the labeling moieties for the differential hybridization format.

Signals gathered from nucleic acid arrays can be analyzed using commercially available software, such as those provide by Affymetrix or Agilent Technologies. Controls, such as for scan sensitivity, probe labeling and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

The gene expression data collected from nucleic acid arrays can be correlated with clinical outcome using a variety of methods. Suitable correlation methods include, but are not limited to, statistical methods (such as Spearman's rank correlation, Cox proportional hazard regression model, ANOVA/t test, or other suitable rank tests or survival models) and class-based correlation metrics (such as nearest-neighbor analysis).

In one aspect, class-based correlation metrics are used to identify the correlation between peripheral blood gene expression and clinical outcome. In one embodiment, patients with a specified solid tumor are divided into at least two classes based on their clinical stratifications. The correlation between peripheral blood gene expression (e.g., in PBMCs) and clinical outcome is analyzed by a supervised cluster algorithm. Exemplary supervised clustering algorithms include, but are not limited to, nearest-neighbor analysis, support vector machines, and SPLASH. Under the supervised cluster algorithms, clinical outcome of each class of patients is either known or determinable. Genes that are differentially expressed in peripheral blood cells (e.g., PBMCs) of one class of patients relative to the other class of patients can be identified. In many cases, the genes thus identified are substantially correlated with a class distinction between the two classes of patients. The genes thus identified can be used as surrogate markers for predicting clinical outcome of the solid tumor in a patient of interest.

In another embodiment, patients with a specified solid tumor can be divided into at least two classes based on gene expression profiles in their peripheral blood cells. Methods suitable for this purpose include unsupervised clustering algorithms, such as self-organized maps (SOMs), k-means, principal component analysis, and hierarchical clustering. A substantial number (e.g., at least 50%, 60%, 70%, 80%, 90%, or more) of patients in one class may have a first clinical outcome, and a substantial number of patients in the other class may have a second clinical outcome. Genes that are differentially expressed in the peripheral blood cells of one class of patients relative to the other class of patients can be identified. These genes are prognosis genes for the solid tumor.

In yet another embodiment, patients with a specified solid tumor can be divided into three or more classes based on their clinical stratifications or peripheral blood gene expression profiles. Multi-class correlation metrics can be employed to identify genes that are differentially expressed in these classes. Exemplary multi-class correlation metrics include, but are not limited to, GeneCluster 2 software provided by MIT Center for Genome Research at Whitehead Institute (Cambridge, Mass.).

In a further embodiment, nearest-neighbor analysis (also known as neighborhood analysis) is used to analyze gene expression data gathered from nucleic acid arrays. The algorithm for neighborhood analysis is described in Golub, et al., SCIENCE, 286: 531-537 (1999), Slonim, et al., PROCS. OF THE FOURTH ANNUAL INTERNATIONAL CONFERENCE ON COMPUTATIONAL MOLECULAR BIOLOGY, Tokyo, Japan, April 8-11, p 263-272 (2000), and U.S. Pat. No. 6,647,341, all of which are incorporated herein by reference. Under one form of the neighborhood analysis, the expression profile of each gene can be represented by an expression vector g=(e₁, e₂, e₃, . . . , eₙ), where eᵢ corresponds to the expression level of gene “g” in the ith sample. A class distinction can be represented by an idealized expression pattern c=(c₁, c₂, c₃, . . . , cₙ), where cᵢ=1 or −1, depending on whether the ith sample is isolated from class 0 or class 1. Class 0 may include patients having a first clinical outcome, and class 1 includes patients having a second clinical outcome. Other forms of class distinction can also be employed. Typically, a class distinction represents an idealized expression pattern, where the expression level of a gene is uniformly high for samples in one class and uniformly low for samples in the other class.

The correlation between gene “g” and the class distinction can be measured by a signal-to-noise score:
P(g,c)=[μ₁(g)−μ₂(g)]/[σ₁(g)+σ₂(g)]
where μ₁(g) and μ₂(g) represent the means of the log-transformed expression levels of gene “g” in class 0 and class 1, respectively, and σ₁(g) and σ₂(g) represent the standard deviation of the log-transformed expression levels of gene “g” in class 0 and class 1, respectively. A higher absolute value of a signal-to-noise score indicates that the gene is more highly expressed in one class than in the other. In one embodiment, the samples used to derive the signal-to-noise score comprise enriched or purified PBMCs. Thus, the signal-to-noise score P(g,c) can represent a correlation between the class distinction and the expression level of gene “g” in PBMCs.

The correlation between gene “g” and the class distinction can also be measured by other methods, such as by the Pearson correlation coefficient or the Euclidean distance, as appreciated by those skilled in the art.

The significance of the correlation between peripheral blood gene expression patterns and the class distinction can be evaluated using a random permutation test. An unusually high density of genes within the neighborhoods of the class distinction, as compared to random patterns, suggests that many genes have expression patterns that are significantly correlated with the class distinction. The correlation between genes and the class distinction can be diagrammatically viewed through a neighborhood analysis plot, in which the y-axis represents the number of genes within various neighborhoods around the class distinction and the x-axis indicates the size of the neighborhood (i.e., P(g,c)). Curves showing different significance levels for the number of genes within corresponding neighborhoods of randomly permuted class distinctions can also be included in the plot.

In one embodiment, the prognosis genes of the present invention are substantially correlated with a class distinction between two outcome classes. In one example, the prognosis genes are above the median significance level in the neighborhood analysis plot. This means that the correlation measure P(g,c) for each prognosis gene is such that the number of genes within the neighborhood of the class distinction having the size of P(g,c) is greater than the number of genes within the corresponding neighborhoods of randomly permuted class distinctions at the median significance level. In another example, the employed prognosis genes are above the 10%, 5%, 2%, or 1% significance level. As used herein, x % significance level means that x % of random neighborhoods contain as many genes as the real neighborhood around the class distinction.

Class predictors can be constructed using the prognosis genes of the present invention. These class predictors are useful for assigning class membership to solid tumor patients. In one embodiment, the prognosis genes in a class predictor are limited to those shown to be significantly correlated with the class distinction by the permutation test, such as those at above the 1%, 2%, 5%, 10%, 20%, 30%, 40%, or 50% significance level. In another embodiment, the expression level of each prognosis gene in a class predictor is substantially higher or substantially lower in PBMCs of one class of patients than in the other class of patients. In still another embodiment, the prognosis genes in a class predictor have top absolute values of P(g,c). In yet another embodiment, the p-value under a Student's t-test (e.g., two-tailed distribution, two sample unequal variance) for each differentially expressed prognosis gene is no more than 0.05, 0.01, 0.005, 0.001, 0.0005, 0.0001, or less.

In a further embodiment, the class predictors of the present invention have at least 50% accuracy for leave-one-out cross validation. In another embodiment, the class predictors of the present invention have at least 60%, 70%, 80%, 90%, 95%, or 99% accuracy for leave-one-out cross validation.

In another aspect, the correlation between peripheral blood gene expression profiles and clinical outcome can be evaluated by statistical methods. Clinical outcome suitable for these analyses includes, but are not limited to, TTP, TTD, and other time-associated clinical indicators. One exemplary statistical method employs Spearman's rank correlation coefficient, which has the formula of:
rₛ=SS_{UV}/(SS_{UU}SS_{VV})^{1/2}
where SS_{UV}=ΣUᵢVᵢ−[(ΣUᵢ)(ΣVᵢ)]/n, SS_{UU}=ΣVᵢ²−[(ΣVᵢ)²]/n, and SS_{VV}=ΣUᵢ²−[ΣUᵢ)²]/n. Uᵢ is the expression level ranking of a gene of interest, Vᵢ is the ranking of the clinical outcome, and n represents the number of patients. The shortcut formula for Spearman's rank correlation coefficient is rₛ=1−(6×Σdᵢ²)/[n(n²−1)], where dᵢ=Uᵢ−Vᵢ. The Spearman's rank correlation is similar to the Pearson's correlation except that it is based on ranks and is thus more suitable for data that is not normally distributed. See, for example, Snedecor and Cochran, STATISTICAL METHODS, Eight edition, Iowa State University Press, Ames, Iowa, 503 pp, 1989. The correlation coefficient is tested to assess whether it differs significantly from a value of 0 (i.e., no correlation).

The correlation coefficients for each prognosis gene identified by the Spearman's rank correlation can be either positive or negative, provided that the correlation is statistically significant. In many embodiments, the p-value for each prognosis gene thus identified is no more than 0.05, 0.01, 0.005, 0.001, 0.0005, 0.0001, or less. In many other embodiments, the Spearman correlation coefficients of the prognosis genes thus identified have absolute values of at least 0.3, 0.4, 0.5, 0.6, 0.7, 0.8, 0.9, or more.

Another exemplary statistical method is Cox proportional hazard regression model, which has the formula of:
log hᵢ(t)=α(t)+βⱼxᵢⱼ
where hᵢ(t) is the hazard function that assesses the instantaneous risk of demise at time t, conditional on survival to that time, α(t) is the baseline hazard function, and xᵢⱼ is a covariate which may represent, for example, the expression level of prognosis gene j in a peripheral blood sample. See Cox, JOURNAL OF THE ROYAL STATISTICAL SOCIETY, SERIES B 34:187 (1972). Additional covariates, such as interactions between covariates, can also be included in Cox proportional hazard model. As used herein, the terms “demise” or “survival” are not limited to real death or survival. Instead, these terms should be interpreted broadly to cover any type of time-associated events, such as TTP. In many cases, the p-values for the correlation under Cox proportional hazard regression model are no more than 0.05, 0.01, 0.005, 0.001, 0.0005, 0.0001, or less. The p-values for the prognosis genes identified under Cox proportional hazard regression model can be determined by the likelihood ratio test, Wald test, the Score test, or the log-rank test. In one embodiment, the hazard ratios for the prognosis genes thus identified are at least 1.5, 2, 3, 4, 5, or more. In another embodiment, the hazard ratios for the prognosis genes thus identified are no more than 0.67, 0.5., 0.33, 0.25., 0.2, or less.

Other rank tests, scores, measurements, or models can also be employed to identify prognosis genes whose expression profiles in peripheral blood samples are correlated with clinical outcome of solid tumors. These tests, scores, measurements, or models can be either parametric or nonparametric, and the regression may be either linear or non-linear. Many statistical methods and correlation/regression models can be carried out using commercially available programs.

Other methods capable of identifying genes differentially expressed in peripheral blood cells of one class of patients relative to another class of patients can be used. These methods include, but are not limited, RT-PCR, Northern Blot, in situ hybridization, and immunoassays such as ELISA, RIA or Western Blot. The expression levels of genes thus identified can be substantially higher or substantially lower in peripheral blood cells (e.g., PBMCs) of one class of patients than in another class of patients. In some cases, the average peripheral blood expression level of a prognosis gene in PBMCs of one class of patients can be at least 2, 3, 4, 5, 10, 20, or more folds higher or lower than that in another class of patients. In many embodiments, the p-value of an appropriate statistical significance test (e.g., Student's t-test) for the difference between average expression levels is no more than 0.05, 0.01, 0.005, 0.001, 0.0005, 0.0001, or less.

Prognosis genes for other non-blood diseases can be similarly identified according to the present invention, provided that the correlation between peripheral blood gene expression and clinical outcome of these diseases, is statistically significant. The peripheral blood expression patterns of the prognosis genes thus identified are indicative of clinical outcome of these diseases.

II. Identification of RCC Prognosis Genes

RCC comprises the majority of all cases of kidney cancer and is one of the ten most common cancers in industrialized countries, comprising 2% of adult malignancies and 2% of cancer-related deaths. Several prognostic factors and scoring indices have been developed for patients diagnosed with RCC, typified by multivariate assessments of several key indicators. As an example, one prognostic scoring system employs the five prognostic factors proposed by Motzer, et al., supra—namely, Karnofsky performance status, serum lactate dehydrognease, hemoglobin, serum calcium, and presence/absence of prior nephrectomy.

The present invention identifies numerous RCC prognosis genes whose peripheral blood expression profiles correlate with patient outcome in CCI-779 therapy. In a clinical trial, the cytostatic mTOR inhibitor CCI-779 was evaluated in RCC patients for its anti-cancer effect. PBMCs collected prior to CCI-779 therapy were analyzed on oligonucleotide arrays in order to determine whether mononuclear cells from RCC patients possessed transcriptional patterns predictive of patient outcome. The results of both supervised and unsupervised analyses indicated that transcriptional profiles in the surrogate tissue of PBMCs from RCC patients prior to treatment with CCI-779 are significantly correlated with patient outcome.

PBMCs were isolated prior to CCI-779 therapy from peripheral blood of 45 advanced RCC patients (18 females and 27 males) participating in a phase 2 clinical trial study. Written informed consent for the pharmacogenomic portion of the clinical study was received for all individuals and the project was approved by the local Institutional Review Boards at the participating clinical sites. RCC tumors of patients were classified at the clinical sites as conventional (clear cell) carcinomas (24), granular (1), papillary (3), or mixed subtypes (7). Ten tumors were classified as unknown. RCC patients were primarily of Caucasian descent (44 Caucasian, 1 African-American) and had a mean age of 58 years (range of 40-78 years). Inclusion criteria included patients with histologically confirmed advanced renal cancer who had received prior therapy for advanced disease, or who had not received prior therapy for advanced disease but were not appropriate candidates to receive high doses of IL-2 therapy. Other inclusion criteria included patients with (1) bi-dimensionally measurable evidence of disease; (2) evidence of progression of the disease prior to study entry; (3) an age of 18 years or older; (4) ANC>1500/μL, platelet>100,000/μL and hemoglobin>8.5 g/dL; (5) adequate renal function evidenced by serum creatinine<1.5× upper limit of normal; (6) adequate hepatic function evidenced by biliruubin<1.5× upper limit of normal and AST<3× upper limit of normal (or AST<5× upper limit of normal if liver metastases were present); (7) serum cholesterol<350 mg/dL, triglycerides<300 mg/dL; (8) ECOG performance status 0-1; and (9) a life expectancy of at least 12 weeks. Exclusion criteria included patients who had (1) the presence of known CNS metastases; (2) surgery or radiotherapy within 3 weeks of start of dosing; (3) chemotherapy or biologic therapy for RCC within 4 weeks of start of dosing; (4) treatment with a prior investigational agent within 4 weeks of start of dosing; (5) immunocompromised status including those known to be HIV positive, or receiving concurrent use of immunosuppressive agents including corticosteroids; (6) active infections; (7) required treatment with anticonvulsant therapy; (8) presence of unstable angina/myocardial infarction within 6 months/ongoing treatment of life-threatening arrythmia; (9) history of prior malignancy in past 3 years; (10) hypersensitivity to macrolide antibiotics; and (11) pregnancy or any other illness which would substantially increase the risk associated with participation in the study.

These advanced RCC patients were treated with one of 3 doses of CCI-779 (25 mg, 75 mg, or 250 mg) administered as a 30 minute intravenous (IV) infusion once weekly for the duration of the trial. CCI-779 is an ester analog of the immunosuppressant rapamycin and as such is a potent, selective inhibitor of the mammalian target of rapamycin. The mammalian target of rapamycin (mTOR) activates multiple signaling pathways, including phosphorylation of p70s6kinase, which results in increased translation of 5′ TOP mRNAs encoding proteins involved in translation and entry into the G1 phase of the cell cycle. By virtue of its inhibitory effects on mTOR and cell cycle control, CCI-779 functions as a cytostatic and immunosuppressive agent.

Clinical staging and size of residual, recurrent or metastatic disease were recorded prior to treatment and every 8 weeks following initiation of CCI-779 therapy. Tumor size was measured in centimeters and reported as the product of the longest diameter and its perpendicular. Measurable disease was defined as any bidimensionally measurable lesion where both diameters>1.0 cm by CT-scan, X-ray or palpation. Tumor response was determined by the sum of the products of all measurable lesions. The categories for assignment of clinical response were given by the clinical protocol definitions (i.e., progressive disease, stable disease, minor response, partial response, and complete response). The category for assignment of prognosis under the Motzer risk assessment (favorable vs intermediate vs poor) was also used. Among the 45 RCC patients, 6 were assigned a favorable risk assessment, 17 patients possessed an intermediate risk score, and 22 patients received a poor prognosis classification. In addition to the categorical classifications, overall survival and time to disease progression were also monitored as clinical endpoints.

HgU95A genechips (manufactured by Affymetrix) were used to detect baseline expression profiles in PBMCs of the RCC patients prior to the CCI-779 therapy. Each HgU95A genechip comprises over 12,600 human sequences according to the Affymetrix Expression Analysis Technical Manual. RNA transcripts were first isolated from PBMCs of the RCC patients. cRNA was then prepared and hybridized to the genechips according to protocols described in the Affymetrix's Expression Analysis Technical Manual. Hybridization signals were collected, scaled, and normalized before being subject to further analysis. In one example, the log of the expression level for each gene was normalized across the samples such that the mean is zero and the standard deviation is one.

The expression profiling analysis revealed that of the 12,626 genes on the HgU95A chip, 5,424 genes met the initial criteria (i.e., at least 1 present call across the data set and at least 1 frequency≧10 ppm). On average, 4,023 transcripts were detected as “present” in any given RCC PBMC profile.

In an initial assessment of the expression data in baseline PBMCs, pairwise correlations were calculated to assess the association between gene expression levels measured by HgU95A Affymetrix microarrays and continuous measures of clinical outcome. Correlations were run using expression levels from each of 5,424 qualifiers that passed the initial criteria. Correlations were run for two clinical measures (TTD and TTP) and for one measure of baseline expression level (log₂-transformed scaled frequency in units of ppm).

In one example, Spearman's rank correlations were computed. The p-value for the hypothesis that the correlation was equal to 0 was calculated for each pairwise correlation. For each comparison between clinical outcome and gene expression, the number of tests that were nominally significant out of the 5,424 tests performed was calculated for five Type I (i.e. false-positive) error levels. To adjust for the fact that 5,424 non-independent tests were performed, a permutation-based approach was employed to evaluate how often the observed number of significance tests would be found under the null hypothesis of no correlation.

The overall results for Spearman's rank correlation comparisons of clinical outcome with baseline expression levels (log₂-transformed scaled frequency) are summarized in Tables 2a and 2b. Each table shows alpha confidence levels (“α”), the observed numbers of transcripts that have nominally significant Spearman correlations with the clinical outcome of interest (“Observed Number”), and the percentage of permutations for which number of nominally significant Spearman correlations equals or exceeds the number observed (“%-age of Permutations”). Evidence for association between clinical outcome and baseline gene expression in PBMCs was significant for both TTD and TTP.

TABLE 2a
Spearman Correlations of Clinical Outcome with Baseline
Expression Levels in PBMCs of RCC Patients in CCI-779
Therapy (n = 45 patients)
Time to Disease Progression

| | Observed Number of | |
|---|---|---|
| | Nominally | %-age of Permutations for |
| | Significant | which Number of Nominally Significant |
| | Spearman | Spearman Correlations |
| α | Correlations* | equals or exceeds observed number |
| 0.1 | 1127 | 5.3% (53/1000) |
| 0.05 | 749 | 3.8% (38/1000) |
| 0.01 | 248 | 3.1% (31/1000) |
| 0.005 | 159 | 2.6% (26/1000) |
| 0.001 | 51 | 2.5% (25/1000) |

*based on 5,424 genes (filtered by at least one Present and at least one frequency ≧ 10 ppm)

TABLE 2b
Spearman Correlations of Clinical Outcome with Baseline
Expression Levels in PBMCs of RCC Patients in CCI-779
Therapy (n = 45 patients)
Time to Death

| | | %-age of Permutations |
|---|---|---|
| | Observed Number of | for which Number of Nominally |
| | Nominally | Significant Spearman Correlations |
| | Significant Spearman | equals or exceeds observed |
| α | Correlations* | number |
| 0.1 | 1604 | 0.1% (1/1000) |
| 0.05 | 1117 | 0.1% (1/1000) |
| 0.01 | 436 | 0.1% (1/1000) |
| 0.005 | 289 | 0.1% (1/1000) |
| 0.001 | 105 | 0.3% (3/1000) |

*based on 5,424 genes (filtered by at least one Present and at least one frequency ≧ 10 ppm)

Table 3 lists the results of the Spearman's rank correlation analyses for all of the 5,424 genes that met the initial criteria. Each gene has a corresponding qualifier on the HgU95A genechip, and each qualifier represents multiple oligonucleotide probes that are stably attached to discrete regions on the HgU95A genechip. According to the design, RNA transcripts of a gene, or the complements thereof, are expected to hybridize under nucleic acid array hybridization conditions to the corresponding qualifier on the HgU95A genechip. As used herein, a polynucleotide can hybridize to a qualifier if the polynucleotide, or the complement thereof, can hybridize to at least one oligonucleotide probe of the qualifier. In many embodiments, the polynucleotide or the complement thereof can hybridize to at least 50%, 60%, 70%, 80%, 90% or 100% of all of the oligonucleotide probes of the qualifier.

Each gene or qualifier in Table 3 may have a corresponding SEQ ID NO or Entrez accession number from which the oligonucleotide probes of the qualifier can be derived. In many instances, a polypeptide capable of hybridizing to a qualifier can also hybridize to the sequence of the corresponding SEQ ID NO or Entrez accession number, or the complement thereof. The sequence of each Entrez accession number can be obtained from the Entrez nucleotide database at the National Center of Biotechnology Information (NCBI). The Entrez nucleotide database collects sequences from several sources, including GenBank, RefSeq, and PDB. Each SEQ ID NO may be derived from the sequence of the corresponding Entrez accession number. Table 4 shows the Entrez and Unigene accession numbers for all of the qualifiers on the HgU95A genechip that met the initial criteria.

Any ambiguous residue (“n”) in a SEQ ID NO can be determined by a variety of methods. In one embodiment, the ambiguous residues in a SEQ ID NO are determined by aligning the SEQ ID NO to a corresponding genomic sequence obtained from a human genome sequence database. In another embodiment, the ambiguous residues in a SEQ ID NO are determined based on the sequence of the corresponding Entrez accession number. In yet another embodiment, the ambiguous residues are determined by re-sequencing the SEQ ID NO.

Genes associated with each qualifier on the HgU95A genechip can be identified based on the annotations provided by Affymetrix. All of the genes thus identified are listed in Tables 3 and 5. These genes can also be identified based on their corresponding Entrez or Unigene accession numbers. In addition, these genes can be determined by BLAST searching their corresponding SEQ ID NOs, or the unambiguous segments thereof, against a human genome sequence database. Suitable human genome sequence databases for this purpose include, but are not limited to, the NCBI human genome database. The NCBI provides BLAST programs, such as “blastn,” for searching its sequence databases.

In one embodiment, the BLAST search of the NCBI human genome database is carried out by using an unambiguous segment (e.g., the longest unambiguous segment) of a SEQ ID NO. Gene(s) that aligns to the unambiguous segment with significant sequence identity can be identified. In many cases, the identified gene(s) has at least 95%, 96%, 97%, 98%, 99%, or more sequence identity with the unambiguous segment.

On the basis of Spearman's rank correlation, prognosis genes that are highly correlated with TTP or TTD were identified. Table 6a lists examples of genes whose expression levels are positively correlated with TTP. Table 6b depicts examples of genes whose expression levels are negatively correlated with TTP. Table 6c provides examples of genes whose expression levels are positively correlated with TTD. Table 6d shows examples of genes whose expression levels are negatively correlated with TTD. Correlation coefficients, p-values, and the corresponding qualifiers are also indicated for each gene in Tables 6a, 6b, 6c, and 6d.

TABLE 6a
Prognosis Genes Positively Correlated with TTP

| | HgU95A | Correlation | P- | |
|---|---|---|---|---|
| SEQ ID NO: | Qualifier | Coefficient | Value | Gene Name |
| 757 | 38518_at | 0.6019 | 0.0000 | SCML2 |
| 1835 | 37343_at | 0.5932 | 0.0000 | ITPR3 |
| 318 | 41174_at | 0.5925 | 0.0000 | RANBP2L1 |
| 755 | 41669_at | 0.5908 | 0.0000 | KIAA0191 |
| 1619 | 40584_at | 0.5602 | 0.0001 | NUP88 |
| 1734 | 41767_r_at | 0.5591 | 0.0001 | KIAA0855 |
| 158 | 38256_s_at | 0.5551 | 0.0001 | DKFZP564O092 |
| 1122 | 39829_at | 0.5508 | 0.0001 | ARL7 |
| 1275 | 35802_at | 0.5475 | 0.0001 | KIAA1014 |
| 452 | 32169_at | 0.5407 | 0.0001 | KIAA0875 |
| 263 | 41562_at | 0.5272 | 0.0002 | BMI1 |
| 39 | 35753_at | 0.5226 | 0.0002 | PRP8 |
| 238 | 40905_s_at | 0.5223 | 0.0002 | DKFZP566J153 |
| 373 | 41547_at | 0.5189 | 0.0003 | BUB3 |
| 1372 | 37416_at | 0.5177 | 0.0003 | ARHH |
| 237 | 37585_at | 0.5157 | 0.0003 | SNRPA1 |
| 483 | 34716_at | 0.5143 | 0.0003 | TASR |
| 603 | 32183_at | 0.5034 | 0.0004 | SFRS11 |
| 768 | 39426_at | 0.4977 | 0.0005 | CA150 |
| 1633 | 35815_at | 0.4975 | 0.0005 | HYPB |
| 1253 | 36403_s_at | 0.4972 | 0.0005 | UNK_AI434146 |
| 225 | 40828_at | 0.4963 | 0.0005 | P85SPR |
| 1119 | 35364_at | 0.4947 | 0.0006 | APPBP1 |
| 423 | 33861_at | 0.4931 | 0.0006 | UNK_AI123426 |
| 231 | 36474_at | 0.4927 | 0.0006 | KIAA0776 |
| 346 | 35764_at | 0.4908 | 0.0006 | CXORF5 |
| 840 | 39129_at | 0.4904 | 0.0006 | UNK_AF052134 |
| 248 | 32508_at | 0.4893 | 0.0006 | KIAA1096 |
| 935 | 35842_at | 0.4862 | 0.0007 | UNK_AL049265 |
| 278 | 41737_at | 0.4862 | 0.0007 | SRM160 |
| 120 | 36303_f_at | 0.4833 | 0.0008 | ZNF85 |
| 223 | 34256_at | 0.4829 | 0.0008 | SIAT9 |
| 79 | 33845_at | 0.4828 | 0.0008 | HNRPH1 |
| 808 | 40048_at | 0.4822 | 0.0008 | UNK_D43951 |
| 77 | 37625_at | 0.4801 | 0.0008 | IRF4 |
| 535 | 33234_at | 0.4779 | 0.0009 | UNK_AA887480 |
| 1689 | 2000_at | 0.4777 | 0.0009 | ATM |
| 820 | 37078_at | 0.4760 | 0.0010 | CD3Z |
| 427 | 38778_at | 0.4744 | 0.0010 | KIAA1046 |

TABLE 6b
Prognosis Genes Negatively Correlated with TTP

| | HgU95A | Correlation | P- | |
|---|---|---|---|---|
| SEQ ID NO: | Qualifier | Coefficient | Value | Gene Name |
| 5299 | 935_at | −0.6319 | 0.0000 | CAP |
| 4559 | 34498_at | −0.5385 | 0.0001 | VNN2 |
| 5296 | 37023_at | −0.5292 | 0.0002 | LCP1 |
| 5303 | 286_at | −0.5189 | 0.0003 | H2AFO |
| 5200 | 38831_f_at | −0.5152 | 0.0003 | UNK_AF053356 |
| 4988 | 268_at | −0.5126 | 0.0003 | PECAM1 |
| 5059 | 38893_at | −0.5006 | 0.0005 | NCF4 |
| 4509 | 34319_at | −0.4950 | 0.0005 | S100P |
| 4899 | 37328_at | −0.4931 | 0.0006 | PLEK |
| 4664 | 181_g_at | −0.4925 | 0.0006 | UNK_S82470 |
| 4939 | 38894_g_at | −0.4852 | 0.0007 | NCF4 |
| 5208 | 32736_at | −0.4805 | 0.0008 | UNK_W68830 |

TABLE 6c
Prognosis Genes Positively Correlated with TTD

| | HgU95A | Correlation | P- | |
|---|---|---|---|---|
| SEQ ID NO: | Qualifier | Coefficient | Value | Gene Name |
| 16 | 37385_at | 0.6524 | 0.0000 | CYP |
| 1 | 41606_at | 0.6155 | 0.0000 | DRG1 |
| 271 | 33420_g_at | 0.6043 | 0.0000 | API5 |
| 5 | 35353_at | 0.5969 | 0.0000 | PSMC2 |
| 614 | 38017_at | 0.5942 | 0.0000 | CD79A |
| 213 | 31851_at | 0.5854 | 0.0000 | RFP2 |
| 27 | 35319_at | 0.5817 | 0.0000 | CTCF |
| 11 | 38702_at | 0.5702 | 0.0000 | UNK_AF070640 |
| 231 | 36474_at | 0.5654 | 0.0001 | KIAA0776 |
| 223 | 34256_at | 0.5649 | 0.0001 | SIAT9 |
| 201 | 34763_at | 0.5575 | 0.0001 | CSPG6 |
| 129 | 33831_at | 0.5561 | 0.0001 | CREBBP |
| 33 | 229_at | 0.5499 | 0.0001 | CBF2 |
| 43 | 37381_g_at | 0.5478 | 0.0001 | GTF2B |
| 1261 | 40092_at | 0.5436 | 0.0001 | BAZ2A |
| 145 | 39746_at | 0.5428 | 0.0001 | POLR2B |
| 318 | 41174_at | 0.5424 | 0.0001 | RANBP2L1 |
| 248 | 32508_at | 0.5397 | 0.0001 | KIAA1096 |
| 90 | 33403_at | 0.5390 | 0.0001 | DKFZP547E1010 |
| 110 | 39809_at | 0.5381 | 0.0001 | HBP1 |
| 303 | 34829_at | 0.5373 | 0.0001 | DKC1 |
| 77 | 37625_at | 0.5350 | 0.0002 | IRF4 |
| 38 | 35656_at | 0.5336 | 0.0002 | RNF6 |
| 450 | 39509_at | 0.5328 | 0.0002 | UNK_AI692348 |
| 53 | 33543_s_at | 0.5324 | 0.0002 | PNN |
| 398 | 38082_at | 0.5318 | 0.0002 | KIAA0650 |
| 120 | 36303_f_at | 0.5311 | 0.0002 | ZNF85 |
| 154 | 1885_at | 0.5300 | 0.0002 | ERCC3 |
| 284 | 32194_at | 0.5285 | 0.0002 | CBF2 |
| 605 | 41621_i_at | 0.5264 | 0.0002 | ZNF266 |
| 76 | 33151_s_at | 0.5239 | 0.0002 | UNK_W25932 |
| 452 | 32169_at | 0.5212 | 0.0002 | KIAA0875 |
| 32 | 36845_at | 0.5203 | 0.0002 | KIAA0136 |
| 771 | 36231_at | 0.5197 | 0.0003 | UNK_AC002073 |
| 85 | 35163_at | 0.5172 | 0.0003 | KIAA1041 |
| 238 | 40905_s_at | 0.5170 | 0.0003 | DKFZP566J153 |
| 202 | 39431_at | 0.5164 | 0.0003 | NPEPPS |
| 755 | 41669_at | 0.5160 | 0.0003 | KIAA0191 |
| 61 | 35294_at | 0.5150 | 0.0003 | SSA2 |
| 1195 | 39401_at | 0.5139 | 0.0003 | UNK_W28264 |
| 483 | 34716_at | 0.5137 | 0.0003 | TASR |
| 82 | 40563_at | 0.5136 | 0.0003 | DKFZP564A043 |
| 255 | 38667_at | 0.5124 | 0.0003 | UNK_AA189161 |
| 197 | 38122_at | 0.5107 | 0.0003 | SLC23A1 |
| 237 | 37585_at | 0.5096 | 0.0004 | SNRPA1 |
| 603 | 32183_at | 0.5079 | 0.0004 | SFRS11 |
| 114 | 40816_at | 0.5074 | 0.0004 | PWP1 |
| 13 | 33818_at | 0.5055 | 0.0004 | UNK_AC004472 |
| 193 | 37703_at | 0.5042 | 0.0004 | RABGGTB |
| 2 | 38016_at | 0.5039 | 0.0004 | HNRPD |
| 56 | 37737_at | 0.4997 | 0.0005 | PCMT1 |
| 24 | 36872_at | 0.4976 | 0.0005 | ARPP-19 |
| 12 | 39415_at | 0.4975 | 0.0005 | HNRPK |
| 7 | 40252_g_at | 0.4970 | 0.0005 | HRB2 |
| 48 | 39727_at | 0.4966 | 0.0005 | DUSP11 |
| 6 | 1728_at | 0.4966 | 0.0005 | BMI1 |
| 296 | 34967_at | 0.4956 | 0.0005 | UNK_AF001549 |
| 69 | 39864_at | 0.4949 | 0.0005 | CIRBP |
| 22 | 32758_g_at | 0.4947 | 0.0006 | RAE1 |
| 39 | 35753_at | 0.4943 | 0.0006 | PRP8 |
| 188 | 1857_at | 0.4916 | 0.0006 | MADH7 |
| 346 | 35764_at | 0.4915 | 0.0006 | CXORF5 |
| 1045 | 32372_at | 0.4911 | 0.0006 | CTSB |
| 17 | 33485_at | 0.4892 | 0.0006 | RPL4 |
| 10 | 34647_at | 0.4887 | 0.0007 | DDX5 |
| 468 | 1442_at | 0.4886 | 0.0007 | ESR2 |
| 97 | 41506_at | 0.4875 | 0.0007 | MAPKAPK5 |
| 118 | 34879_at | 0.4873 | 0.0007 | DPM1 |
| 1795 | 39512_s_at | 0.4869 | 0.0007 | UNK_AA457029 |
| 66 | 36783_f_at | 0.4865 | 0.0007 | H-PLK |
| 485 | 35479_at | 0.4860 | 0.0007 | ADAM28 |
| 151 | 40308_at | 0.4858 | 0.0007 | UNK_AI830496 |
| 442 | 38462_at | 0.4852 | 0.0007 | NDUFA5 |
| 347 | 781_at | 0.4851 | 0.0007 | RABGGTB |
| 60 | 38102_at | 0.4850 | 0.0007 | UNK_W28575 |
| 158 | 38256_s_at | 0.4829 | 0.0008 | DKFZP564O092 |
| 332 | 32850_at | 0.4817 | 0.0008 | NUP153 |
| 94 | 35286_r_at | 0.4815 | 0.0008 | RY1 |
| 49 | 36456_at | 0.4815 | 0.0008 | DKFZP564I052 |
| 362 | 38924_s_at | 0.4813 | 0.0008 | SSH3BP1 |
| 190 | 35805_at | 0.4809 | 0.0008 | DKFZP434D156 |
| 195 | 40086_at | 0.4805 | 0.0008 | KIAA0261 |
| 847 | 34274_at | 0.4801 | 0.0008 | KIAA1116 |
| 322 | 39897_at | 0.4793 | 0.0009 | DDX16 |
| 405 | 41665_at | 0.4792 | 0.0009 | KIAA0824 |
| 52 | 38114_at | 0.4785 | 0.0009 | RAD21 |
| 1490 | 41166_at | 0.4782 | 0.0009 | IGHM |
| 182 | 41569_at | 0.4781 | 0.0009 | KIAA0974 |
| 517 | 33440_at | 0.4774 | 0.0009 | TCF8 |
| 582 | 36459_at | 0.4767 | 0.0009 | KIAA0879 |
| 414 | 216_at | 0.4765 | 0.0009 | PTGDS |
| 389 | 41199_s_at | 0.4760 | 0.0009 | SFPQ |
| 1694 | 40051_at | 0.4756 | 0.0010 | KIAA0057 |
| 578 | 38019_at | 0.4754 | 0.0010 | CSNK1E |
| 565 | 36690_at | 0.4746 | 0.0010 | NR3C1 |
| 373 | 41547_at | 0.4742 | 0.0010 | BUB3 |
| 269 | 38105_at | 0.4734 | 0.0010 | UNK_W26521 |
| 225 | 40828_at | 0.4732 | 0.0010 | P85SPR |
| 509 | 41809_at | 0.4729 | 0.0010 | UNK_AI656421 |
| 8 | 36210_g_at | 0.4727 | 0.0010 | FSRG1 |

TABLE 6d
Prognosis Genes Negatively Correlated with TTD

| | HgU95A | Correlation | | |
|---|---|---|---|---|
| SEQ ID NO: | Qualifier | Coefficient | P-Value | Gene Name |
| 5303 | 286_at | −0.5871 | 0.0000 | H2AFO |
| 5279 | 32609_at | −0.5841 | 0.0000 | H2AFO |
| 5312 | 38483_at | −0.5464 | 0.0001 | HSA011916 |
| 5281 | 769_s_at | −0.5036 | 0.0004 | ANXA2 |
| 5286 | 1131_at | −0.4876 | 0.0007 | MAP2K2 |
| 5024 | 32378_at | −0.4818 | 0.0008 | PKM2 |
| | 956_at | −0.4770 | 0.0009 | TUBB |
| 5185 | 37311_at | −0.4760 | 0.0010 | TALDO1 |
| 5220 | 37148_at | −0.4744 | 0.0010 | LILRB3 |
| 5272 | 36199_at | −0.4725 | 0.0010 | DAP |

In addition to the specific genes described herein, the present invention contemplates the use of any other gene that can hybridize under stringent or nucleic acid array hybridization conditions to a qualifier identified in the present invention. These genes may include hypothetical or putative genes that are supported by EST or mRNA data. The expression profiles of these genes may correlate with patient clinical outcome. As used herein, a gene can hybridize to a qualifier if an RNA transcript of the gene can hybridize to at least one oligonucleotide probe of the qualifier. In many cases, an RNA transcript of the gene can hybridize to at least 50%, 60%, 70%, 80%, 90%, or more oligonucleotide probes of the qualifier.

The oligonucleotide probe sequences of each qualifier on HgU95A genechips may be obtained from Affymetrix or from the sequence files maintained at Affymetrix website “www.affymetrix.com/support/technical/byproduct.affx?product=hgu95sequence.” For instance, the oligonucleotide probe sequences can be found in the sequence file “HG_U95A Probe Sequences, FASTA” at the website. This sequence file is incorporated herein by reference in its entirety.

In another example, a Cox proportional hazard regression model was employed to assess the correlation between baseline PBMC gene expression levels and clinical outcome. Cox model can take into account the effects of censoring on correlations of gene expression with TTD (or Survival as of last known date alive) and TTP (or progression-free status as of last known date alive). Of the 45 RCC patients with baseline PBMC expression levels, 4 had censored data for TTP and 15 had censored data for TTD. Similar to the Spearman's assessment of the data, Cox regression can identify genes significantly correlated with survival and disease progression for any given α-confidence level. A similar permutation strategy can be used to affirm any correlation between baseline expression profiles and clinical outcome.

In one embodiment, models were fit using expression levels from each of the 5,424 qualifiers that passed the initial filtering criteria in the 45 baseline samples. TTP and TTD were tested for their association with log2-transformed scaled frequency at baseline. A SAS program was used to generate the estimates in Tables 7a and 7b. Tables 7a and 7b demonstrate a strong correlation between TTP/TTD and baseline gene expression.

TABLE 7a
Cox Regressions of Clinical Outcome on Baseline Expression
Levels in PBMCs of RCC Patients in CCI-779 Therapy
(n = 45 patients)
Time to Progression

| | | Percentage of Permutations for |
|---|---|---|
| | | which Number of Nominally |
| | Observed Number of | Significant Cox Regressions |
| | Nominally Significant | Equals or Exceeds Observed |
| ∀ | Cox Regressions* | Number** |
| 0.1 | 1439 | 0.8% (4/500) |
| 0.05 | 950 | 0.8% (3/500) |
| 0.01 | 342 | 0.8% (4/500) |
| 0.005 | 217 | 0.8% (4/500) |
| 0.001 | 53 | 1.0% (5/500) |

*for 5,424 genes (filtered by at least one Present call and at least one frequency ≧ 10 ppm)
**based on 500 random permutations

TABLE 7b
Cox Regressions of Clinical Outcome on Baseline Expression
Levels in PBMCs of RCC Patients in CCI-779 Therapy
(n = 45 patients)
Time to Death

| | | Percentage of Permutations for |
|---|---|---|
| | | which Number of Nominally |
| | Observed Number of | Significant Cox Regressions |
| | Nominally Significant | Equals or Exceeds Observed |
| ∀ | Cox Regressions* | Number** |
| 0.1 | 1948 | <0.2% (0/500) |
| 0.05 | 1383 | <0.2% (0/500) |
| 0.01 | 602 | <0.2% (0/500) |
| 0.005 | 404 | <0.2% (0/500) |
| 0.001 | 142 | <0.2% (0/500) |

*for 5,424 genes (filtered by at least one Present call and at least one frequency ≧ 10 ppm)
**based on 500 random permutations

Table 8 lists the results of Cox proportional hazard modeling for all of the 5,424 genes that met the initial criteria. Hazard ratios and p-values (for the hypothesis that the risk coefficient was equal to 1, i.e., no risk) are indicated for each gene. Examples of genes that are indicative of high risk for TTP or TTD are shown in Tables 9a or 9c, respectively. These genes have hazard ratios of at least 3. Examples of genes that are indicative of low risk for TTP or TTD are described in Tables 9b or 9d, respectively. These genes have hazard ratios of no more than 0.333.

TABLE 9a
Prognosis Genes Indicative of High Risk for TTP

| | | Hazard | | |
|---|---|---|---|---|
| SEQ ID NO: | HgU95A Qualifier | Ratio | P-Value | Gene Name |
| 5296 | 37023_at | 6.1066 | 0.0001 | LCP1 |
| 5299 | 935_at | 5.8829 | 0.0000 | CAP |
| 5315 | 40771_at | 4.9503 | 0.0586 | MSN |
| 5313 | 37298_at | 4.6595 | 0.0046 | GABARAP |
| 5235 | 31820_at | 4.2099 | 0.0061 | HCLS1 |
| 5248 | 676_g_at | 4.1051 | 0.0016 | IFITM1 |
| 4920 | 33906_at | 3.9750 | 0.0106 | SSSCA1 |
| 5208 | 32736_at | 3.8093 | 0.0013 | UNK_W68830 |
| 4958 | 40169_at | 3.5692 | 0.0243 | TIP47 |
| 5314 | 39811_at | 3.4197 | 0.1074 | UNK_AA402538 |
| 5275 | 1309_at | 3.3680 | 0.0053 | PSMB3 |
| 4524 | 39814_s_at | 3.2703 | 0.0029 | UNK_AI052724 |
| 5153 | 38605_at | 3.1625 | 0.0592 | NDUFB1 |
| 5200 | 38831_f_at | 3.0853 | 0.0092 | UNK_AF053356 |

TABLE 9b
Prognosis Genes Indicative of Low Risk for TTP

| | | Hazard | | |
|---|---|---|---|---|
| SEQ ID NO: | HgU95A Qualifier | Ratio | P-Value | Gene Name |
| 12 | 39415_at | 0.0818 | 0.0002 | HNRPK |
| 39 | 35753_at | 0.1608 | 0.0001 | PRP8 |
| 282 | 33667_at | 0.1650 | 0.0890 | PPIA |
| 79 | 33845_at | 0.1657 | 0.0024 | HNRPH1 |
| 26 | 36186_at | 0.1661 | 0.0040 | RNPS1 |
| 241 | 1420_s_at | 0.1662 | 0.0009 | EIF4A2 |
| 127 | 31950_at | 0.1724 | 0.0071 | PABPC1 |
| 10 | 34647_at | 0.1831 | 0.0010 | DDX5 |
| 459 | 36515_at | 0.2094 | 0.0002 | GNE |
| 230 | 36111_s_at | 0.2147 | 0.0031 | SFRS2 |
| 1287 | 39180_at | 0.2154 | 0.0009 | FUS |
| 22 | 32758_g_at | 0.2186 | 0.0010 | RAE1 |
| 15 | 31952_at | 0.2211 | 0.0076 | RPL6 |
| 81 | 38527_at | 0.2258 | 0.0016 | NONO |
| 232 | 32831_at | 0.2298 | 0.0006 | TIM17 |
| 19 | 37609_at | 0.2321 | 0.0016 | NUBP1 |
| 18 | 34695_at | 0.2330 | 0.0035 | GA17 |
| 157 | 39730_at | 0.2331 | 0.0005 | ABL1 |
| 689 | 35808_at | 0.2385 | 0.0037 | SFRS6 |
| 246 | 32751_at | 0.2386 | 0.0013 | UNK_AF007140 |
| 278 | 41737_at | 0.2393 | 0.0023 | SRM160 |
| 338 | 32205_at | 0.2431 | 0.0009 | PRKRA |
| 7 | 40252_g_at | 0.2473 | 0.0033 | HRB2 |
| 54 | 35325_at | 0.2540 | 0.0030 | UNK_AF052113 |
| 1823 | 41292_at | 0.2549 | 0.0014 | HNRPH1 |
| 2174 | 32658_at | 0.2553 | 0.0010 | UNK_AL031228 |
| 242 | 33307_at | 0.2569 | 0.0008 | UNK_AL022316 |
| 35 | 40426_at | 0.2587 | 0.0306 | BCL7B |
| 263 | 41562_at | 0.2595 | 0.0010 | BMI1 |
| 9 | 34315_at | 0.2638 | 0.0149 | AFG3L2 |
| 5046 | 33920_at | 0.2665 | 0.0549 | DIAPH1 |
| 105 | 33706_at | 0.2698 | 0.0114 | SART1 |
| 3048 | 35170_at | 0.2706 | 0.0053 | MAN2C1 |
| 33 | 229_at | 0.2715 | 0.0064 | CBF2 |
| 17 | 33485_at | 0.2724 | 0.0169 | RPL4 |
| 6 | 1728_at | 0.2736 | 0.0103 | BMI1 |
| 269 | 38105_at | 0.2748 | 0.0017 | UNK_W26521 |
| 58 | 1361_at | 0.2801 | 0.0059 | TERF1 |
| 528 | 32171_at | 0.2831 | 0.0040 | EIF5 |
| 49 | 36456_at | 0.2834 | 0.0015 | DKFZP564I052 |
| 2420 | 838_s_at | 0.2841 | 0.0616 | UBE2I |
| 1527 | 1706_at | 0.2852 | 0.0144 | ARAF1 |
| 427 | 38778_at | 0.2882 | 0.0012 | KIAA1046 |
| 93 | 39378_at | 0.2896 | 0.1463 | BECN1 |
| 162 | 34225_at | 0.2911 | 0.0126 | UNK_AF101434 |
| 1029 | 32833_at | 0.2918 | 0.0016 | CLK1 |
| 898 | 34285_at | 0.2938 | 0.0021 | KIAA0795 |
| 205 | 35743_at | 0.2968 | 0.0133 | NAR |
| 1735 | 39165_at | 0.2971 | 0.0086 | NIFU |
| 361 | 36685_at | 0.2979 | 0.0045 | AMD1 |
| 285 | 37557_at | 0.2985 | 0.0038 | SLC4A2 |
| 120 | 36303_f_at | 0.2987 | 0.0018 | ZNF85 |
| 3896 | 33392_at | 0.3019 | 0.0030 | DKFZP434J154 |
| 335 | 40160_at | 0.3031 | 0.0038 | DKFZP586P2220 |
| 606 | 34337_s_at | 0.3047 | 0.0009 | M96 |
| 585 | 37506_at | 0.3053 | 0.0006 | UNK_Z78308 |
| 158 | 38256_s_at | 0.3053 | 0.0002 | DKFZP564O092 |
| 324 | 37690_at | 0.3053 | 0.0120 | ILVBL |
| 59 | 1020_s_at | 0.3060 | 0.0069 | SIP2-28 |
| 1606 | 36862_at | 0.3066 | 0.0147 | KIAA1115 |
| 176 | 39141_at | 0.3069 | 0.0074 | ABCF1 |
| 167 | 32592_at | 0.3071 | 0.0280 | KIAA0323 |
| 340 | 39044_s_at | 0.3076 | 0.0141 | DGKD |
| 133 | 40596_at | 0.3076 | 0.0058 | TCOF1 |
| 1186 | 34369_at | 0.3078 | 0.0454 | KIAA0214 |
| 1189 | 33188_at | 0.3090 | 0.0006 | PPIL2 |
| 1536 | 41220_at | 0.3110 | 0.0404 | MSF |
| 844 | 38445_at | 0.3125 | 0.0057 | ARHGEF1 |
| 66 | 36783_f_at | 0.3125 | 0.0064 | H-PLK |
| 166 | 37717_at | 0.3126 | 0.0130 | NAGR1 |
| 174 | 36198_at | 0.3167 | 0.0058 | KIAA0016 |
| 42 | 35125_at | 0.3171 | 0.0540 | RPS6 |
| 1227 | 32438_at | 0.3172 | 0.0557 | RPS20 |
| 236 | 37030_at | 0.3181 | 0.0006 | KIAA0887 |
| 193 | 37703_at | 0.3183 | 0.0011 | RABGGTB |
| 1577 | 1711_at | 0.3199 | 0.0463 | TP53BP1 |
| 461 | 41691_at | 0.3216 | 0.0006 | KIAA0794 |
| 587 | 32079_at | 0.3219 | 0.0037 | KIAA0639 |
| 725 | 39865_at | 0.3230 | 0.0151 | UNK_AI890903 |
| 160 | 34326_at | 0.3232 | 0.0025 | COPB |
| 1157 | 34808_at | 0.3244 | 0.0188 | KIAA0999 |
| 981 | 36129_at | 0.3244 | 0.0014 | UNK_AB007857 |
| 50 | 37672_at | 0.3249 | 0.0077 | USP7 |
| 71 | 32208_at | 0.3257 | 0.0098 | KIAA0355 |
| 215 | 35298_at | 0.3266 | 0.0973 | EIF3S7 |
| 290 | 36982_at | 0.3267 | 0.0018 | USP14 |
| 1308 | 31573_at | 0.3292 | 0.0566 | RPS25 |
| 307 | 36603_at | 0.3292 | 0.0015 | GCN1L1 |
| 4 | 36189_at | 0.3310 | 0.0661 | ILF2 |
| 68 | 39155_at | 0.3325 | 0.0433 | PSMD3 |

TABLE 9c
Prognosis Genes Indicative of High Risk for TTD

| SEQ ID NO: | HgU95A Qualifier | Hazard Ratio | P-Value | Gene Name |
|---|---|---|---|---|
| 5315 | 40771_at | 9.6763 | 0.0122 | MSN |
| 5314 | 39811_at | 8.0370 | 0.0149 | UNK_AA402538 |
| 5313 | 37298_at | 7.6453 | 0.0021 | GABARAP |
| 5312 | 38483_at | 6.7764 | 0.0001 | HSA011916 |
| 5311 | 1878_g_at | 6.1122 | 0.0004 | ERCC1 |
| 5310 | 33994_g_at | 4.9451 | 0.0009 | MYL6 |
| 5309 | 32318_s_at | 4.9169 | 0.0027 | ACTB |
| 5308 | 37012_at | 4.8396 | 0.0057 | CAPZB |
| 5307 | 1199_at | 4.7016 | 0.0103 | EIF4A1 |
| 5306 | 36641_at | 4.5981 | 0.0042 | CAPZA2 |
| 5305 | 34160_at | 4.5693 | 0.0086 | ACTG1 |
| 5304 | 34091_s_at | 4.4114 | 0.0158 | VIM |
| 5303 | 286_at | 4.2492 | 0.0000 | H2AFO |
| 5302 | 35770_at | 4.1617 | 0.0083 | ATP6S1 |
| 5301 | 33341_at | 4.0632 | 0.0102 | GNB1 |
| 5300 | 33659_at | 4.0505 | 0.0074 | CFL1 |
| 5299 | 935_at | 4.0159 | 0.0016 | CAP |
| 5298 | 40134_at | 3.8316 | 0.0043 | ATP5J2 |
| 5297 | 37346_at | 3.8205 | 0.0126 | ARF5 |
| 5296 | 37023_at | 3.8170 | 0.0059 | LCP1 |
| 5295 | 38451_at | 3.8077 | 0.0034 | UQCR |
| 5294 | 34836_at | 3.7786 | 0.0080 | RABL |
| 5293 | 35263_at | 3.6729 | 0.0558 | EIF4EBP2 |
| 5292 | 41724_at | 3.6595 | 0.0026 | DXS1357E |
| 5291 | 33679_f_at | 3.5643 | 0.0134 | TUBB2 |
| 5290 | 33121_g_at | 3.5151 | 0.0007 | RGS10 |
| 5289 | 40872_at | 3.4884 | 0.0013 | COX6B |
| 5288 | 1315_at | 3.4428 | 0.0026 | UNK_D78361 |
| 5287 | 36574_at | 3.4083 | 0.1032 | IDH3G |
| 5286 | 1131_at | 3.3872 | 0.0002 | MAP2K2 |
| 5285 | 31444_s_at | 3.3199 | 0.0016 | ANXA2P2 |
| 5284 | 36963_at | 3.3124 | 0.0060 | PGD |
| 5283 | 35083_at | 3.2546 | 0.0517 | UNK_AL031670 |
| 5282 | 32145_at | 3.2308 | 0.0012 | ADD1 |
| | AFFX- | 3.1377 | 0.0060 | BACTIN3_Hs_AFFX |
| | HSAC07/X00351_3_at | | | |
| 5281 | 769_s_at | 3.1358 | 0.0006 | ANXA2 |
| 5280 | 35783_at | 3.0738 | 0.0592 | UNK_H93123 |
| 5279 | 32609_at | 3.0361 | 0.0000 | H2AFO |
| 5278 | 1695_at | 3.0329 | 0.0225 | NEDD8 |

TABLE 9d
Prognosis Genes Indicative of Low Risk for TTD

| SEQ | | Hazard | | |
|---|---|---|---|---|
| ID NO: | HgU95A Qualifier | Ratio | P-Value | Gene Name |
| 1 | 41606_at | 0.0322 | 0.0000 | DRG1 |
| 2 | 38016_at | 0.0547 | 0.0003 | HNRPD |
| 3 | 39274_at | 0.1030 | 0.0004 | NUP62 |
| 4 | 36189_at | 0.1100 | 0.0029 | ILF2 |
| 5 | 35353_at | 0.1140 | 0.0000 | PSMC2 |
| 6 | 1728_at | 0.1250 | 0.0001 | BMI1 |
| 7 | 40252_g_at | 0.1265 | 0.0003 | HRB2 |
| 8 | 36210_g_at | 0.1287 | 0.0003 | FSRG1 |
| 9 | 34315_at | 0.1288 | 0.0028 | AFG3L2 |
| 10 | 34647_at | 0.1295 | 0.0001 | DDX5 |
| 11 | 38702_at | 0.1333 | 0.0000 | UNK_AF070640 |
| 12 | 39415_at | 0.1428 | 0.0019 | HNRPK |
| 13 | 33818_at | 0.1433 | 0.0011 | UNK_AC004472 |
| 14 | 37509_at | 0.1447 | 0.0001 | UNK_AF046059 |
| 15 | 31952_at | 0.1466 | 0.0025 | RPL6 |
| 16 | 37385_at | 0.1538 | 0.0000 | CYP |
| 17 | 33485_at | 0.1591 | 0.0010 | RPL4 |
| 18 | 34695_at | 0.1620 | 0.0013 | GA17 |
| 19 | 37609_at | 0.1625 | 0.0004 | NUBP1 |
| 20 | 32807_at | 0.1675 | 0.0012 | DKFZP566C134 |
| 21 | 33614_at | 0.1694 | 0.0017 | RPL18A |
| 22 | 32758_g_at | 0.1727 | 0.0010 | RAE1 |
| 23 | 32766_at | 0.1742 | 0.0056 | G22P1 |
| 24 | 36872_at | 0.1763 | 0.0001 | ARPP-19 |
| 25 | 34401_at | 0.1764 | 0.0095 | UQCRFS1 |
| 26 | 36186_at | 0.1791 | 0.0047 | RNPS1 |
| 27 | 35319_at | 0.1792 | 0.0000 | CTCF |
| 28 | 755_at | 0.1796 | 0.0023 | ITPR1 |
| 29 | 40370_f_at | 0.1809 | 0.0104 | HLA-G |
| 30 | 37353_g_at | 0.1824 | 0.0013 | SP100 |
| 31 | 41295_at | 0.1825 | 0.0005 | GPX3 |
| 32 | 36845_at | 0.1886 | 0.0001 | KIAA0136 |
| 33 | 229_at | 0.1887 | 0.0008 | CBF2 |
| 34 | 39766_r_at | 0.1906 | 0.0016 | POLR2K |
| 35 | 40426_at | 0.1909 | 0.0183 | BCL7B |
| 36 | 38456_s_at | 0.1912 | 0.0240 | UNK_AL049650 |
| 37 | 35595_at | 0.1945 | 0.0000 | CGRP-RCP |
| 38 | 35656_at | 0.1945 | 0.0001 | RNF6 |
| 39 | 35753_at | 0.1955 | 0.0014 | PRP8 |
| 40 | 37367_at | 0.1965 | 0.0429 | ATP6E |
| 41 | 38590_r_at | 0.1981 | 0.0171 | PTMA |
| 42 | 35125_at | 0.2004 | 0.0120 | RPS6 |
| 43 | 37381_g_at | 0.2014 | 0.0003 | GTF2B |
| 44 | 36946_at | 0.2024 | 0.0004 | DYRK1A |
| 45 | 38068_at | 0.2027 | 0.0010 | AMFR |
| 46 | 32175_at | 0.2049 | 0.0156 | CDC10 |
| 47 | 31538_at | 0.2057 | 0.0031 | RPLP0 |
| 48 | 39727_at | 0.2079 | 0.0003 | DUSP11 |
| 49 | 36456_at | 0.2120 | 0.0003 | DKFZP564I052 |
| 50 | 37672_at | 0.2121 | 0.0013 | USP7 |
| 51 | 41288_at | 0.2154 | 0.0060 | CALM1 |
| 52 | 38114_at | 0.2167 | 0.0036 | RAD21 |
| 53 | 33543_s_at | 0.2190 | 0.0002 | PNN |
| 54 | 35325_at | 0.2193 | 0.0043 | UNK_AF052113 |
| 55 | 39562_at | 0.2197 | 0.0018 | CGGBP1 |
| 56 | 37737_at | 0.2226 | 0.0004 | PCMT1 |
| 57 | 33740_at | 0.2241 | 0.0061 | UNK_AF023268 |
| 58 | 1361_at | 0.2250 | 0.0030 | TERF1 |
| 59 | 1020_s_at | 0.2250 | 0.0020 | SIP2-28 |
| 60 | 38102_at | 0.2281 | 0.0001 | UNK_W28575 |
| 61 | 35294_at | 0.2308 | 0.0003 | SSA2 |
| 62 | 40700_at | 0.2309 | 0.0022 | SP140 |
| 63 | 39020_at | 0.2310 | 0.0067 | SIVA |
| 64 | 1449_at | 0.2311 | 0.0025 | PSMA4 |
| 65 | 34821_at | 0.2319 | 0.0007 | DKFZP586D0623 |
| 66 | 36783_f_at | 0.2319 | 0.0010 | H-PLK |
| 67 | 39740_g_at | 0.2329 | 0.0085 | NACA |
| 68 | 39155_at | 0.2333 | 0.0138 | PSMD3 |
| 69 | 39864_at | 0.2344 | 0.0002 | CIRBP |
| 70 | 39099_at | 0.2361 | 0.0011 | SEC23A |
| 71 | 32208_at | 0.2365 | 0.0036 | KIAA0355 |
| 72 | 39027_at | 0.2377 | 0.0174 | COX4 |
| 73 | 39774_at | 0.2390 | 0.0207 | OXA1L |
| 74 | 40449_at | 0.2391 | 0.0006 | RFC1 |
| 75 | 40369_f_at | 0.2395 | 0.0154 | UNK_AL022723 |
| 76 | 33151_s_at | 0.2407 | 0.0002 | UNK_W25932 |
| 77 | 37625_at | 0.2410 | 0.0000 | IRF4 |
| 78 | 35055_at | 0.2415 | 0.0223 | BTF3 |
| 79 | 33845_at | 0.2416 | 0.0065 | HNRPH1 |
| 80 | 33451_s_at | 0.2418 | 0.0128 | RPL22 |
| 81 | 38527_at | 0.2425 | 0.0064 | NONO |
| 82 | 40563_at | 0.2425 | 0.0001 | DKFZP564A043 |
| 83 | 36975_at | 0.2427 | 0.0037 | UNK_W26659 |
| 84 | 38854_at | 0.2445 | 0.0037 | KIAA0635 |
| 85 | 35163_at | 0.2485 | 0.0001 | KIAA1041 |
| 86 | 38817_at | 0.2492 | 0.0087 | SPAG7 |
| 87 | 41787_at | 0.2502 | 0.0004 | KIAA0669 |
| 88 | 649_s_at | 0.2504 | 0.0001 | CXCR4 |
| 89 | 37715_at | 0.2510 | 0.0002 | SNW1 |
| 90 | 33403_at | 0.2511 | 0.0000 | DKFZP547E1010 |
| 91 | 34172_s_at | 0.2512 | 0.0013 | UNK_M99578 |
| 92 | 32576_at | 0.2522 | 0.0151 | EIF3S5 |
| 93 | 39378_at | 0.2550 | 0.1231 | BECN1 |
| 94 | 35286_r_at | 0.2554 | 0.0009 | RY1 |
| 95 | 37350_at | 0.2559 | 0.0102 | UNK_AL031177 |
| 96 | 38123_at | 0.2559 | 0.0025 | D123 |
| 97 | 41506_at | 0.2559 | 0.0001 | MAPKAPK5 |
| 98 | 40140_at | 0.2559 | 0.0004 | ZFP103 |
| 99 | 38073_at | 0.2561 | 0.0018 | RNMT |
| 100 | 31872_at | 0.2563 | 0.0029 | SSXT |
| 101 | 34349_at | 0.2564 | 0.0035 | SEC63L |
| 102 | 39792_at | 0.2568 | 0.0002 | HNRPR |
| 103 | 35187_at | 0.2578 | 0.0061 | UNK_AL080216 |
| 104 | 1220_g_at | 0.2578 | 0.0003 | IRF2 |
| 105 | 33706_at | 0.2584 | 0.0209 | SART1 |
| 106 | 34809_at | 0.2588 | 0.0102 | KIAA0999 |
| 107 | 39342_at | 0.2588 | 0.0499 | MARS |
| 108 | 40874_at | 0.2593 | 0.0541 | EDF1 |
| 109 | 40814_at | 0.2597 | 0.0009 | IDS |
| 110 | 39809_at | 0.2597 | 0.0000 | HBP1 |
| 111 | 37226_at | 0.2599 | 0.0014 | BNIP1 |
| 112 | 34370_at | 0.2604 | 0.0020 | ARCN1 |
| 113 | 40651_s_at | 0.2604 | 0.0010 | CRHR1 |
| 114 | 40816_at | 0.2607 | 0.0004 | PWP1 |
| 115 | 35195_at | 0.2613 | 0.0051 | RPC |
| 116 | 40110_at | 0.2621 | 0.0108 | IDH3B |
| 117 | 33886_at | 0.2625 | 0.0019 | SSH3BP1 |
| 118 | 34879_at | 0.2639 | 0.0015 | DPM1 |
| 119 | 36968_s_at | 0.2660 | 0.0019 | OIP2 |
| 120 | 36303_f_at | 0.2669 | 0.0006 | ZNF85 |
| 121 | 40219_at | 0.2670 | 0.0103 | HIS1 |
| 122 | 38942_r_at | 0.2670 | 0.0105 | UNK_W28610 |
| 123 | 32487_s_at | 0.2672 | 0.0061 | KPNA4 |
| 124 | 36754_at | 0.2675 | 0.0001 | ADCYAP1 |
| 125 | 39739_at | 0.2683 | 0.0496 | MYH9 |
| 126 | 33443_at | 0.2687 | 0.0004 | UNK_Z99129 |
| 127 | 31950_at | 0.2687 | 0.0321 | PABPC1 |
| 128 | 39059_at | 0.2689 | 0.0145 | DHCR7 |
| 129 | 33831_at | 0.2702 | 0.0001 | CREBBP |
| 130 | 35368_at | 0.2703 | 0.0006 | ZNF207 |
| 131 | 35227_at | 0.2706 | 0.0057 | RBBP8 |
| 132 | 41296_s_at | 0.2713 | 0.0009 | GPX3 |
| 133 | 40596_at | 0.2717 | 0.0047 | TCOF1 |
| 134 | 35910_f_at | 0.2720 | 0.0113 | MMPL1 |
| 135 | 34018_at | 0.2722 | 0.0014 | COL19A1 |
| 136 | 36949_at | 0.2722 | 0.0033 | CSNK1D |
| 137 | 33394_at | 0.2730 | 0.0011 | DDX19 |
| 138 | 34231_at | 0.2734 | 0.0036 | UNK_AF074606 |
| 139 | 32288_r_at | 0.2738 | 0.0014 | KLRC3 |
| 140 | 38903_at | 0.2742 | 0.0007 | GJB5 |
| 141 | 38040_at | 0.2743 | 0.0093 | SPF30 |
| 142 | 39126_at | 0.2749 | 0.0043 | UNK_AL080101 |
| 143 | 35321_at | 0.2752 | 0.0034 | TLK2 |
| 144 | 36546_r_at | 0.2755 | 0.0142 | UNK_AB011114 |
| 145 | 39746_at | 0.2755 | 0.0000 | POLR2B |
| 146 | 41256_at | 0.2762 | 0.0054 | EEF1D |
| 147 | 41789_r_at | 0.2781 | 0.0012 | KIAA0669 |
| 148 | 35630_at | 0.2784 | 0.0025 | LLGL2 |
| 149 | 40984_at | 0.2789 | 0.0384 | UNK_W28255 |
| 150 | 35199_at | 0.2789 | 0.0035 | KIAA0982 |
| 151 | 40308_at | 0.2791 | 0.0003 | UNK_AI830496 |
| 152 | 40803_at | 0.2793 | 0.0014 | UNK_AL050161 |
| 153 | 322_at | 0.2801 | 0.0045 | PIK3R3 |
| 154 | 1885_at | 0.2804 | 0.0008 | ERCC3 |
| 155 | 193_at | 0.2814 | 0.0330 | TAF2G |
| 156 | 38668_at | 0.2819 | 0.0141 | KIAA0553 |
| 157 | 39730_at | 0.2819 | 0.0088 | ABL1 |
| 158 | 38256_s_at | 0.2821 | 0.0009 | DKFZP564O092 |
| 159 | 39290_f_at | 0.2832 | 0.0013 | DKFZP564M2423 |
| 160 | 34326_at | 0.2833 | 0.0020 | COPB |
| 161 | 38923_at | 0.2838 | 0.0075 | FRG1 |
| 162 | 34225_at | 0.2845 | 0.0092 | UNK_AF101434 |
| 163 | 35258_f_at | 0.2846 | 0.0023 | SFRS2IP |
| 164 | 31546_at | 0.2847 | 0.0090 | RPL18 |
| 165 | 37659_at | 0.2855 | 0.0180 | IMMT |
| 166 | 37717_at | 0.2861 | 0.0090 | NAGR1 |
| 167 | 32592_at | 0.2862 | 0.0215 | KIAA0323 |
| 168 | 35978_at | 0.2871 | 0.0215 | UNK_AF009242 |
| 169 | 31330_at | 0.2873 | 0.0243 | RPS19 |
| 170 | 33388_at | 0.2881 | 0.0289 | UNK_AL080223 |
| 171 | 40036_at | 0.2883 | 0.0041 | MAGOH |
| 172 | 41808_at | 0.2888 | 0.0023 | UNK_AF052102 |
| 173 | 1683_at | 0.2891 | 0.0021 | WIT-1 |
| 174 | 36198_at | 0.2895 | 0.0014 | KIAA0016 |
| 175 | 38689_at | 0.2897 | 0.0146 | DJ149A16.6 |
| 176 | 39141_at | 0.2904 | 0.0053 | ABCF1 |
| 177 | 32593_at | 0.2904 | 0.0090 | KIAA0084 |
| 178 | 32801_at | 0.2914 | 0.0052 | KIAA0317 |
| 179 | 37894_at | 0.2919 | 0.0054 | CUL2 |
| 180 | 38443_at | 0.2921 | 0.0015 | UNK_U79291 |
| 181 | 493_at | 0.2924 | 0.0026 | CSNK1D |
| 182 | 41569_at | 0.2925 | 0.0022 | KIAA0974 |
| 183 | 38455_at | 0.2928 | 0.0066 | UNK_AL049650 |
| 184 | 1660_at | 0.2932 | 0.0010 | UBE2N |
| 185 | 1981_s_at | 0.2932 | 0.0017 | MAX |
| 186 | 31879_at | 0.2942 | 0.0014 | FUBP3 |
| 187 | 38612_at | 0.2944 | 0.0011 | TSPAN-3 |
| 188 | 1857_at | 0.2950 | 0.0002 | MADH7 |
| 189 | 39047_at | 0.2957 | 0.0010 | KIAA0156 |
| 190 | 35805_at | 0.2962 | 0.0028 | DKFZP434D156 |
| 191 | 160_at | 0.2964 | 0.0027 | STAM |
| | 1627_at | 0.2969 | 0.0101 | UNK_Z25437 |
| 192 | 38106_at | 0.2972 | 0.0009 | YR-29 |
| 193 | 37703_at | 0.2973 | 0.0008 | RABGGTB |
| 194 | 35748_at | 0.2982 | 0.0103 | EEF1B2 |
| 195 | 40086_at | 0.2983 | 0.0016 | KIAA0261 |
| 196 | 40103_at | 0.2985 | 0.0053 | VIL2 |
| 197 | 38122_at | 0.2997 | 0.0008 | SLC23A1 |
| 198 | 32590_at | 0.2999 | 0.0113 | NCL |
| 199 | 35254_at | 0.3009 | 0.0040 | FLN29 |
| 200 | 33660_at | 0.3013 | 0.0292 | RPL5 |
| 201 | 34763_at | 0.3015 | 0.0001 | CSPG6 |
| 202 | 39431_at | 0.3016 | 0.0001 | NPEPPS |
| 203 | 41097_at | 0.3019 | 0.0257 | TERF2 |
| 204 | 32352_at | 0.3022 | 0.0045 | PNMT |
| 205 | 35743_at | 0.3029 | 0.0183 | NAR |
| 206 | 39471_at | 0.3036 | 0.0070 | M11S1 |
| 207 | 41413_at | 0.3044 | 0.0131 | CLPTM1 |
| 208 | 1110_at | 0.3048 | 0.0020 | TRD@ |
| 209 | 34600_s_at | 0.3056 | 0.0011 | TUB |
| 210 | 38014_at | 0.3059 | 0.0113 | ADAR |
| 211 | 34215_at | 0.3059 | 0.0131 | DXYS155E |
| 212 | 1017_at | 0.3067 | 0.0048 | MSH6 |
| 213 | 31851_at | 0.3068 | 0.0000 | RFP2 |
| 214 | 34745_at | 0.3071 | 0.1447 | UNK_AF070570 |
| 215 | 35298_at | 0.3073 | 0.1084 | EIF3S7 |
| 216 | 31894_at | 0.3080 | 0.0015 | CENPC1 |
| 217 | 39923_at | 0.3090 | 0.0079 | UNK_AI935420 |
| 218 | 35939_s_at | 0.3097 | 0.0023 | POU4F1 |
| 219 | 1240_at | 0.3098 | 0.0003 | CASP2 |
| 220 | 33661_at | 0.3102 | 0.0017 | RPL5 |
| 221 | 41514_s_at | 0.3105 | 0.0039 | UNK_W26628 |
| 222 | 35186_at | 0.3115 | 0.0016 | PAF65B |
| 223 | 34256_at | 0.3121 | 0.0001 | SIAT9 |
| 224 | 37986_at | 0.3124 | 0.0163 | EPOR |
| 225 | 40828_at | 0.3136 | 0.0010 | P85SPR |
| 226 | 40515_at | 0.3137 | 0.0178 | EIF2B2 |
| 227 | 40277_at | 0.3140 | 0.0022 | KIAA1080 |
| 228 | 1228_s_at | 0.3143 | 0.0070 | MGEA6 |
| 229 | 39917_at | 0.3146 | 0.0341 | GCP2 |
| 230 | 36111_s_at | 0.3146 | 0.0655 | SFRS2 |
| 231 | 36474_at | 0.3157 | 0.0006 | KIAA0776 |
| 232 | 32831_at | 0.3160 | 0.0095 | TIM17 |
| 233 | 1512_at | 0.3161 | 0.0348 | DYRK1A |
| 234 | 38478_at | 0.3162 | 0.0107 | SFRS8 |
| 235 | 38450_at | 0.3167 | 0.0096 | SSB |
| 236 | 37030_at | 0.3170 | 0.0018 | KIAA0887 |
| 237 | 37585_at | 0.3170 | 0.0000 | SNRPA1 |
| 238 | 40905_s_at | 0.3174 | 0.0001 | DKFZP566J153 |
| 239 | 35431_g_at | 0.3177 | 0.0004 | MED6 |
| 240 | 40054_at | 0.3180 | 0.0043 | KIAA0082 |
| 241 | 1420_s_at | 0.3186 | 0.0283 | EIF4A2 |
| 242 | 33307_at | 0.3194 | 0.0073 | UNK_AL022316 |
| 243 | 37984_s_at | 0.3204 | 0.0236 | ARF6 |
| 244 | 41601_at | 0.3205 | 0.0015 | UNK_AA142964 |
| 245 | 38492_at | 0.3206 | 0.0026 | KYNU |
| 246 | 32751_at | 0.3208 | 0.0181 | UNK_AF007140 |
| 247 | 38075_at | 0.3211 | 0.0018 | SYPL |
| 248 | 32508_at | 0.3214 | 0.0008 | KIAA1096 |
| 249 | 38426_at | 0.3220 | 0.0073 | TAF2I |
| 250 | 35327_at | 0.3230 | 0.0203 | EIF3S3 |
| 251 | 1102_s_at | 0.3233 | 0.0037 | NR3C1 |
| 252 | 31463_s_at | 0.3235 | 0.0168 | UNK_AL022097 |
| 253 | 31722_at | 0.3236 | 0.0236 | RPL3 |
| 254 | 1009_at | 0.3237 | 0.0110 | HINT |
| 255 | 38667_at | 0.3239 | 0.0002 | UNK_AA189161 |
| 256 | 36375_at | 0.3244 | 0.0095 | ODF1 |
| 257 | 1793_at | 0.3252 | 0.0049 | CDC2L5 |
| 258 | 41235_at | 0.3256 | 0.1646 | ATF4 |
| 259 | 38816_at | 0.3262 | 0.0006 | TACC2 |
| 260 | 36239_at | 0.3265 | 0.0143 | POU2AF1 |
| 261 | 31951_s_at | 0.3270 | 0.0280 | PABPC1 |
| 262 | 38424_at | 0.3271 | 0.0057 | KIAA0747 |
| 263 | 41562_at | 0.3273 | 0.0033 | BMI1 |
| 264 | 1920_s_at | 0.3277 | 0.0055 | CCNG1 |
| 265 | 35175_f_at | 0.3288 | 0.0125 | EEF1A2 |
| 266 | 40980_at | 0.3288 | 0.0016 | UNK_W26477 |
| 267 | 40833_r_at | 0.3289 | 0.0084 | DKFZP586G011 |
| | 1151_at | 0.3290 | 0.0176 | RPL22 |
| 268 | 32150_at | 0.3294 | 0.0074 | GOLGA4 |
| 269 | 38105_at | 0.3294 | 0.0104 | UNK_W26521 |
| 270 | 32394_s_at | 0.3294 | 0.0249 | RPL23 |
| 271 | 33420_g_at | 0.3297 | 0.0003 | API5 |
| 272 | 39742_at | 0.3298 | 0.0007 | TANK |
| 273 | 32854_at | 0.3303 | 0.0074 | KIAA0696 |
| 274 | 41337_at | 0.3311 | 0.0088 | AES |
| 275 | 35471_g_at | 0.3316 | 0.0113 | HTR2A |
| 276 | 1796_s_at | 0.3322 | 0.0161 | BCL3 |
| 277 | 32541_at | 0.3323 | 0.0013 | PPP3CC |

In another effort, nearest-neighbor analysis was employed to identify multivariate expression patterns in PBMCs of patients that were correlated with clinical responses. This approach included nearest-neighbor-based identification of transcripts most correlated with the class distinction of interest, random permutation of the sample labels to determine the significance of the discovered gene classifiers, and evaluation of the accuracy of various predictive models containing different numbers of genes by leave-one-out cross validation.

In one embodiment, nearest-neighbor analysis and supervised class prediction were performed using Genecluster version 2.0 which has been described by Golub, et al., supra, and is available at www.genome.wi.mit.edu/cancer/software/genecluster2.html. For the analysis, all raw expression data were log transformed and normalized to have a mean value of zero and a variance of one. Class prediction was carried out using a k-nearest-neighbors algorithm as described in Armstrong, et al., NATURE GENETICS, 30:41-47 (2002), which is incorporated herein by reference. This algorithm assigns a test sample to a class by identifying the k-nearest samples in the training set and then choosing the most common class among these k-nearest-neighbors. See Armstrong, et al., supra. For this purpose, distances can be defined by a Euclidean metric on the basis of the expression levels of a specified number of genes.

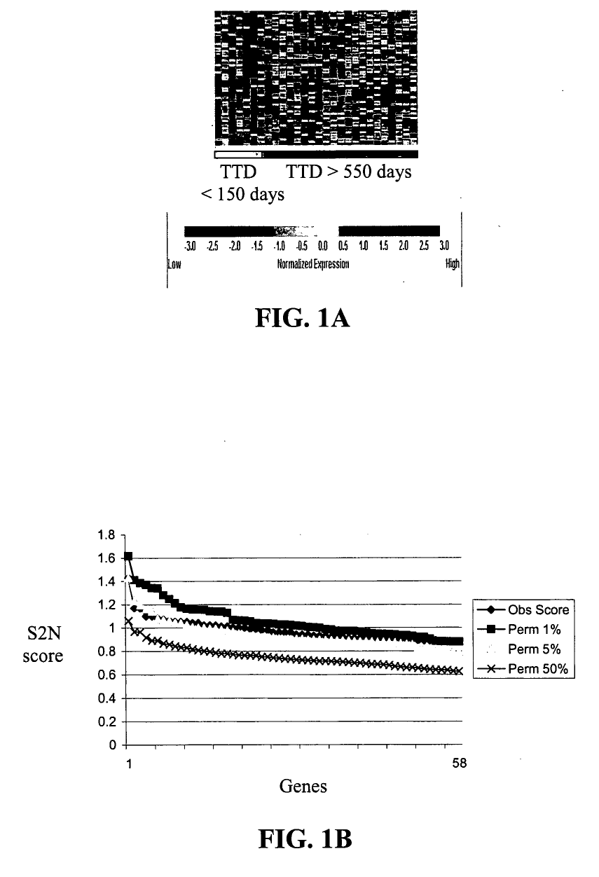

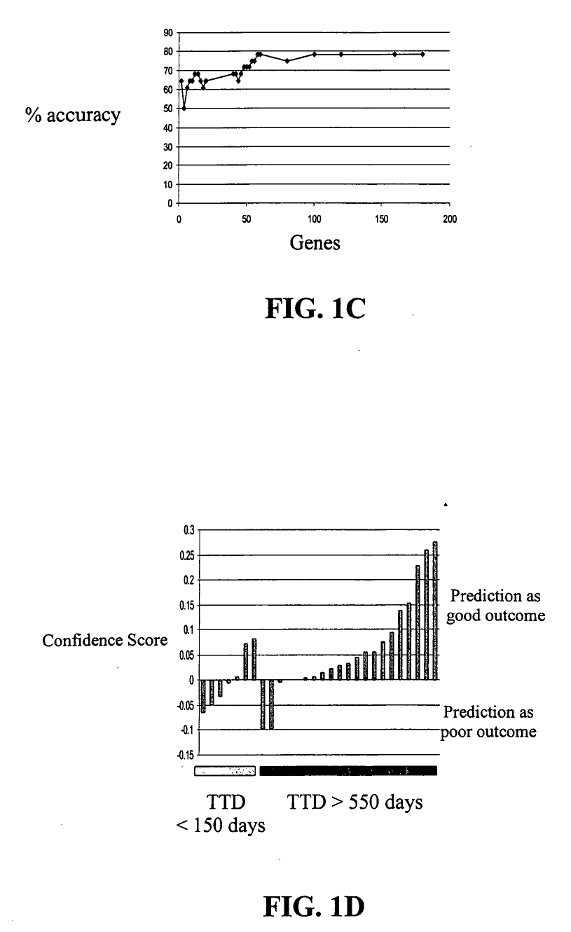

FIGS. 1A-1D illustrate the comparison of short and long term survivors. The class distinction is between RCC patients who had TTD of less than 150 days (the “shorter” class) and RCC patients who had TTD of greater than 550 days (the “longer” class). The relative expression levels of the class-correlated gene (rows in FIG. 1A) were indicated for each patient (columns in FIG. 1A) according to the normalized expression level scale. FIG. 1B depicts the comparison of the signal to noise similarity metric scores (S2N, i.e., |P(g,c)|) for class-correlated genes identified in this clinical stratification relative to S2N scores for the top 1%, 5% and 50% of scores for class-correlated genes resulting from randomly permuted data sets. Examples of the genes that are significantly correlated with the shorter survival-longer survival class distinction are demonstrated in Table 10. Each gene depicted in Table 10 is a prognosis gene and can be used to assign a survival class membership to an RCC patient. Table 10 also shows the HgU95A qualifier for each gene (“Qualifier”), the rank of each gene (“Rank #”), the class within which the gene is more highly expressed (“Class”), the S2N score (“Score”), the S2N score under a random permutation analysis at the 1% significance level (“Perm 1%”), the S2N score under a random permutation analysis at the 5% significance level (“Perm 5%”), and the S2N score under a random permutation analysis at the median significance level (“Perm (user)”). The genes are ranked based on their respective S2N scores. Genes more highly expressed in PBMCs of patients in the “shorter” survival class are ranked from 1 to 29, and genes more highly expressed in PBMCs of patients in the “longer” survival class are ranked from 30 to 58.

TABLE 10
Genes for Predicting Shorter versus Longer Survival

| SEQ ID NO: | Qualifier | Gene Name | Rank # | Class | Score | Perm 1% | Perm 5% | Perm (user) |
|---|---|---|---|---|---|---|---|---|
| 59 | 1020_s_at | SIP2-28 | 35 | Longer | 1.08 | 1.1401024 | 1.0009979 | 0.7793364 |
| | 1665_s_at | ECGF1 | 12 | Shorter | 0.98 | 1.1285181 | 0.9662982 | 0.7793773 |
| 698 | 1815_g_at | TGFBR2 | 38 | Longer | 1.04 | 1.0241055 | 0.9226947 | 0.7515544 |
| 5311 | 1878_g_at | ERCC1 | 27 | Shorter | 0.88 | 0.9426583 | 0.881932 | 0.7000415 |
| 4033 | 214_at | MSX1 | 1 | Shorter | 1.07 | 1.6155937 | 1.4316087 | 1.0612979 |
| 4871 | 31432_g_at | FCGRT | 19 | Shorter | 0.91 | 1.0264453 | 0.9054481 | 0.7332006 |
| 5219 | 32166_at | KIAA1027 | 22 | Shorter | 0.9 | 0.9880754 | 0.8991979 | 0.7198438 |
| 4951 | 32193_at | PLXNC1 | 7 | Shorter | 1 | 1.1596018 | 1.0244524 | 0.834095 |
| 5309 | 32318_s_at | ACTB | 11 | Shorter | 0.98 | 1.1415896 | 0.9838351 | 0.7869063 |
| 4302 | 32475_at | UNK_AF025529 | 10 | Shorter | 0.99 | 1.1436108 | 0.9918097 | 0.7958006 |
| 989 | 32569_at | PAFAH1B1 | 39 | Longer | 1.02 | 1.0132701 | 0.9045167 | 0.7348747 |
| 177 | 32593_at | KIAA0084 | 50 | Longer | 0.91 | 0.9281602 | 0.8635805 | 0.6594012 |
| 20 | 32807_at | DKFZP566C134 | 47 | Longer | 0.92 | 0.9647906 | 0.8758416 | 0.6699242 |
| 76 | 33151_s_at | UNK_W25932 | 46 | Longer | 0.93 | 0.9712016 | 0.8771132 | 0.6791526 |
| 333 | 33354_at | UNK_AA630312 | 56 | Longer | 0.9 | 0.8798124 | 0.794554 | 0.6361411 |
| 126 | 33443_at | UNK_Z99129 | 44 | Longer | 0.94 | 0.9718646 | 0.8817559 | 0.6883464 |
| 5291 | 33679_f_at | TUBB2 | 24 | Shorter | 0.89 | 0.9583792 | 0.8932177 | 0.7133438 |
| 5007 | 33777_at | TBXAS1 | 29 | Shorter | 0.88 | 0.9330735 | 0.8570948 | 0.6878592 |
| 5265 | 33908_at | CAPN1 | 18 | Shorter | 0.93 | 1.0345246 | 0.9114115 | 0.7411601 |
| 4887 | 34033_s_at | LILRA2 | 6 | Shorter | 1.01 | 1.1651943 | 1.0473512 | 0.8420641 |
| 223 | 34256_at | SIAT9 | 53 | Longer | 0.91 | 0.9039352 | 0.7969334 | 0.6420804 |
| 4992 | 34774_at | PPT | 16 | Shorter | 0.94 | 1.0374199 | 0.9192994 | 0.7528306 |
| 357 | 34786_at | KIAA0742 | 32 | Longer | 1.17 | 1.2469592 | 1.0692165 | 0.8567256 |
| 5079 | 34891_at | PIN | 23 | Shorter | 0.9 | 0.9736318 | 0.8943665 | 0.7149921 |
| 1278 | 35268_at | UNK_AL050171 | 49 | Longer | 0.92 | 0.933529 | 0.8717929 | 0.6601154 |
| 5128 | 36091_at | SKAP-HOM | 4 | Shorter | 1.05 | 1.3414925 | 1.0789346 | 0.8906151 |
| 771 | 36231_at | UNK_AC002073 | 31 | Longer | 1.17 | 1.2800804 | 1.1628039 | 0.890024 |
| 1253 | 36403_s_at | UNK_AI434146 | 51 | Longer | 0.91 | 0.9177859 | 0.8269876 | 0.6537137 |
| 571 | 36650_at | CCND2 | 40 | Longer | 1.02 | 1.0060078 | 0.8974235 | 0.7254431 |
| 4885 | 36780_at | CLU | 3 | Shorter | 1.05 | 1.3704714 | 1.1416388 | 0.9158475 |
| 5284 | 36963_at | PGD | 9 | Shorter | 1 | 1.1566645 | 0.9935466 | 0.8085569 |
| 5308 | 37012_at | CAPZB | 21 | Shorter | 0.9 | 1.0171863 | 0.9049488 | 0.7224556 |
| 4860 | 37215_at | PYGL | 25 | Shorter | 0.89 | 0.9504848 | 0.8895108 | 0.711156 |
| 5125 | 37307_at | GNAI2 | 15 | Shorter | 0.96 | 1.0398792 | 0.9262021 | 0.7620184 |
| 43 | 37381_g_at | GTF2B | 57 | Longer | 0.89 | 0.8785508 | 0.7906994 | 0.6284431 |
| 4105 | 37397_at | PECAM1 | 2 | Shorter | 1.06 | 1.4123416 | 1.195739 | 0.9664123 |
| 77 | 37625_at | IRF4 | 33 | Longer | 1.1 | 1.2122538 | 1.0414076 | 0.8297089 |
| 4694 | 37647_at | AOAH | 26 | Shorter | 0.89 | 0.9455904 | 0.8832746 | 0.704616 |
| 4843 | 38397_at | UNK_U09196 | 20 | Shorter | 0.9 | 1.0259999 | 0.9053201 | 0.7286741 |
| 442 | 38462_at | NDUFA5 | 58 | Longer | 0.88 | 0.8780158 | 0.7896803 | 0.6253915 |
| 5238 | 38475_at | DCTN-50 | 13 | Shorter | 0.96 | 1.0638589 | 0.9525263 | 0.7732195 |
| 5312 | 38483_at | HSA011916 | 8 | Shorter | 1 | 1.1577479 | 1.0015978 | 0.8165922 |
| 757 | 38518_at | SCML2 | 45 | Longer | 0.93 | 0.9717825 | 0.8807355 | 0.6834326 |
| 924 | 38589_i_at | PTMA | 52 | Longer | 0.91 | 0.9170299 | 0.8153701 | 0.6481305 |
| 5200 | 38831_f_at | UNK_AF053356 | 5 | Shorter | 1.02 | 1.3394433 | 1.0626743 | 0.864975 |
| 189 | 39047_at | KIAA0156 | 41 | Longer | 1.01 | 1.0031965 | 0.8962379 | 0.7150707 |
| 4971 | 39062_at | PPGB | 17 | Shorter | 0.94 | 1.0372473 | 0.9187932 | 0.7441102 |
| 110 | 39809_at | HBP1 | 36 | Longer | 1.05 | 1.0694007 | 0.9784921 | 0.7662489 |
| 399 | 40610_at | UNK_AI743507 | 42 | Longer | 0.99 | 0.9986351 | 0.8919035 | 0.7074118 |
| 1629 | 40861_at | KIAA0026 | 48 | Longer | 0.92 | 0.9440813 | 0.8742373 | 0.6670547 |
| 5187 | 41045_at | SECTM1 | 28 | Shorter | 0.88 | 0.939004 | 0.8613926 | 0.6939691 |
| 1490 | 41166_at | IGHM | 37 | Longer | 1.04 | 1.0626456 | 0.9303607 | 0.764905 |
| 51 | 41288_at | CALM1 | 43 | Longer | 0.96 | 0.9838136 | 0.8910337 | 0.6987405 |
| 4956 | 41471_at | S100A9 | 14 | Shorter | 0.96 | 1.0545503 | 0.9338488 | 0.7635493 |
| 755 | 41669_at | KIAA0191 | 34 | Longer | 1.1 | 1.1760652 | 1.0059531 | 0.8003741 |
| 2276 | 432_s_at | TRA@ | 55 | Longer | 0.9 | 0.8808494 | 0.7956162 | 0.6383929 |
| 88 | 649_s_at | CXCR4 | 30 | Longer | 1.43 | 1.385432 | 1.2324574 | 0.9647334 |
| 462 | 760_at | DYRK2 | 54 | Longer | 0.9 | 0.8822472 | 0.7956202 | 0.6396517 |

The genes that are significantly correlated with the shorter-longer survival class distinction were used to construct gene classifiers for predicting the survival class membership of an RCC patient. Each predictor set was evaluated by cross validation to identify the predictor set with the highest accuracy for classification of the samples. In these analyses, a 58 gene predictor set (77% accuracy) was identified as the optimal classifier, as shown in FIG. 1C. Table 10 describes these 58 genes. FIG. 1D demonstrates the cross validation results for each sample using the 58-gene predictor. A leave-one-out cross validation was performed and the prediction strengths (PS) were calculated for each sample in the analysis. For the purposes of illustration, confidence scores accompanying calls of “TTD>550 days” were assigned positive values, while prediction strengths accompanying calls of “TTD<150 days” were assigned negative values.

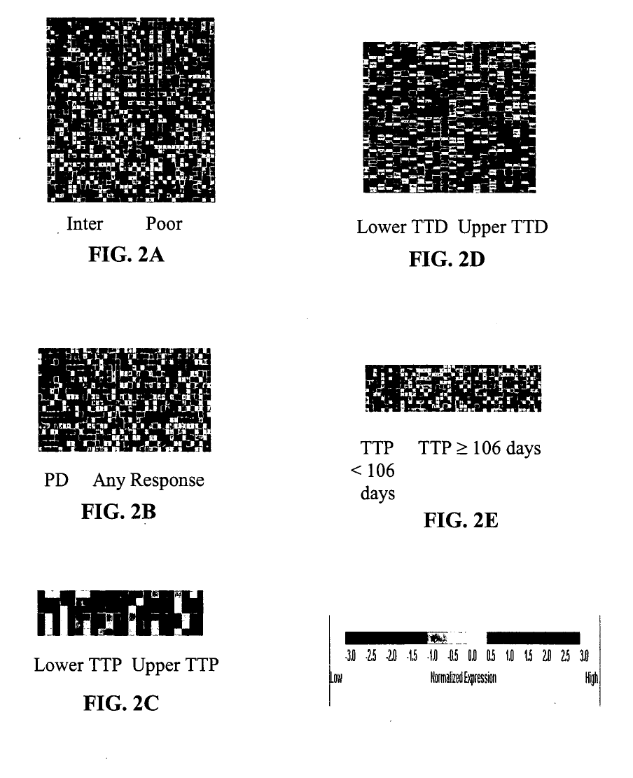

A variety of other clinically relevant stratifications were also performed and relative expression levels of the optimally-sized gene classifiers in each analysis are summarized in FIGS. 2A-2E. The relative expression levels of the genes (rows) in each classifier are indicated for each patient (columns) according to the scale of FIG. 1A. FIG. 2A shows the relative gene expression levels of a 42-gene classifier for the comparison of patients with intermediate versus poor Motzer risk classification. Genes in this classifier are described in Table 11. The baseline expression levels of these genes in PBMCs of RCC patients are predictive of a patient's classification under Motzer risk assessment. FIG. 2B shows the relative gene expression levels for an 18-gene classifier identified in the comparison of patients with progressive disease versus any other clinical response. FIG. 2C demonstrates the relative gene expression levels for a 6-gene classifier identified in the comparison of patients in the lower versus upper quartiles of time to disease progression. Genes in this classifier are illustrated in Table 12. FIG. 2D shows the relative gene expression levels for a 52-gene classifier identified in the comparison of patients in the lower versus upper quartiles of survival/time to death. Finally, FIG. 2E depicts the relative expression levels for a 12-gene classifier identified in the comparison of patients with early (time to disease progression<106 days) versus all other times to disease progression (TTP≧106 days). Genes in this classifier are described in Table 13.

TABLE 11
Prognosis Genes for Intermediate Versus Poor Prognosis Motzer Risk

| SEQ ID NO: | Qualifier | Gene Name | Rank # | Class | Score | Perm 1% | Perm 5% | Perm (user) |
|---|---|---|---|---|---|---|---|---|
| 4924 | 1158_s_at | CALM3 | 23 | Poor | 0.66 | 0.8522128 | 0.8104463 | 0.6502731 |
| 3553 | 31620_at | TBX10 | 39 | Poor | 0.49 | 0.6641291 | 0.6259432 | 0.5179407 |
| 4455 | 31979_at | PFKFB4 | 27 | Poor | 0.62 | 0.7544583 | 0.7037743 | 0.584796 |
| 794 | 31982_at | KIAA0894 | 11 | Intermediate | 0.69 | 0.7164902 | 0.6715787 | 0.5530081 |
| 5083 | 32153_s_at | UNK_U49869 | 42 | Poor | 0.49 | 0.6595597 | 0.6149676 | 0.5025353 |
| 4883 | 32274_r_at | UNK_AF052148 | 35 | Poor | 0.53 | 0.6744095 | 0.6432421 | 0.5315566 |
| 759 | 32530_at | YWHAQ | 6 | Intermediate | 0.74 | 0.7697572 | 0.7312037 | 0.5964533 |
| 92 | 32576_at | EIF3S5 | 17 | Intermediate | 0.67 | 0.6919704 | 0.624558 | 0.5205478 |
| 464 | 32621_at | DR1 | 9 | Intermediate | 0.72 | 0.7232364 | 0.6892603 | 0.5680586 |
| 23 | 32766_at | G22P1 | 18 | Intermediate | 0.67 | 0.6909188 | 0.6235876 | 0.5156429 |
| 3833 | 33178_at | JAG1 | 31 | Poor | 0.54 | 0.716195 | 0.6647701 | 0.554687 |
| 3565 | 33361_at | GNG3LG | 38 | Poor | 0.51 | 0.6721476 | 0.6284547 | 0.5196677 |
| 126 | 33443_at | UNK_Z99129 | 10 | Intermediate | 0.69 | 0.7216778 | 0.680077 | 0.5610381 |
| 4437 | 34430_at | GPT | 25 | Poor | 0.65 | 0.8082772 | 0.7274678 | 0.6092486 |
| 5199 | 34787_at | NRD1 | 37 | Poor | 0.52 | 0.6737965 | 0.6314609 | 0.5246186 |
| 4515 | 35256_at | UNK_AL096737 | 29 | Poor | 0.59 | 0.7415469 | 0.6820045 | 0.5739685 |
| 4688 | 35299_at | MKNK1 | 24 | Poor | 0.65 | 0.8203746 | 0.757703 | 0.6259301 |
| 27 | 35319_at | CTCF | 8 | Intermediate | 0.72 | 0.7329379 | 0.7102606 | 0.5762622 |
| 250 | 35327_at | EIF3S3 | 12 | Intermediate | 0.69 | 0.7115967 | 0.671292 | 0.5470585 |
| 3087 | 36019_at | STK19 | 40 | Poor | 0.49 | 0.6610853 | 0.6217781 | 0.5113894 |
| 4 | 36189_at | ILF2 | 16 | Intermediate | 0.67 | 0.6935341 | 0.6311355 | 0.524226 |
| 5017 | 36391_at | CCNT1 | 32 | Poor | 0.53 | 0.6823648 | 0.6549823 | 0.548012 |
| 4733 | 36956_at | SLC20A2 | 33 | Poor | 0.53 | 0.6811736 | 0.6523389 | 0.5410793 |
| 77 | 37625_at | IRF4 | 21 | Intermediate | 0.65 | 0.6670918 | 0.6195184 | 0.5060937 |
| 2564 | 38064_at | LRP | 41 | Poor | 0.49 | 0.6599081 | 0.6185175 | 0.5034915 |
| 247 | 38075_at | SYPL | 2 | Intermediate | 0.87 | 0.8830003 | 0.8203846 | 0.6704754 |
| 5073 | 38188_s_at | MAN2A2 | 28 | Poor | 0.6 | 0.7427558 | 0.6900191 | 0.5792173 |
| 5123 | 38233_at | HOMER-3 | 30 | Poor | 0.55 | 0.7166691 | 0.6707653 | 0.5600951 |
| 4710 | 38449_at | UNK_W28931 | 36 | Poor | 0.52 | 0.6744089 | 0.635525 | 0.5289256 |
| 183 | 38455_at | UNK_AL049650 | 4 | Intermediate | 0.81 | 0.7940041 | 0.7523503 | 0.6209757 |
| 36 | 38456_s_at | UNK_AL049650 | 5 | Intermediate | 0.75 | 0.7851316 | 0.7383793 | 0.6078528 |
| 5312 | 38483_at | HSA011916 | 22 | Poor | 0.71 | 0.9953936 | 0.8946025 | 0.7231015 |
| 363 | 38738_at | SMT3H1 | 14 | Intermediate | 0.68 | 0.7003638 | 0.6569433 | 0.5350646 |
| 354 | 39057_at | KNS2 | 19 | Intermediate | 0.66 | 0.6841608 | 0.6235478 | 0.5114179 |
| 2976 | 40071_at | CYP1B1 | 7 | Intermediate | 0.73 | 0.7407701 | 0.717859 | 0.5875649 |
| 1918 | 40122_at | NSAP1 | 20 | Intermediate | 0.66 | 0.6713382 | 0.6201956 | 0.5080141 |
| 5255 | 40130_at | FSTL1 | 34 | Poor | 0.53 | 0.6744496 | 0.6458221 | 0.5366854 |
| 411 | 40189_at | SET | 15 | Intermediate | 0.67 | 0.69604 | 0.6381373 | 0.5306426 |
| 415 | 40494_at | DEDD | 13 | Intermediate | 0.68 | 0.7072377 | 0.6653894 | 0.5396373 |
| 399 | 40610_at | UNK_AI743507 | 3 | Intermediate | 0.82 | 0.8709571 | 0.7766898 | 0.6476374 |
| | 727_at | OATL3 | 26 | Poor | 0.63 | 0.7856346 | 0.7178927 | 0.5941055 |
| 2869 | 859_at | CYP1B1 | 1 | Intermediate | 0.88 | 1.0227921 | 0.8774775 | 0.7251933 |

TABLE 12
Prognosis Genes for Lower versus Upper Quartiles of TTP

| SEQ ID NO: | Qualifier | Gene Name | Rank # | Class | Score | Perm 1% | Perm 5% | Perm (user) |
|---|---|---|---|---|---|---|---|---|
| 692 | 32635_at | KIAA1113 | 6 | Upper | 1.16 | 1.3744625 | 1.0978256 | 0.871069 |
| 5007 | 33777_at | TBXAS1 | 3 | Lower | 0.92 | 1.4119021 | 1.1079456 | 0.8730354 |
| 1835 | 37343_at | ITPR3 | 5 | Upper | 1.17 | 1.4312017 | 1.1718279 | 0.9049279 |
| 4829 | 39593_at | FGL2 | 2 | Lower | 0.95 | 1.4426517 | 1.2094518 | 0.9016392 |
| 954 | 41634_at | UNK_D87445 | 4 | Upper | 1.17 | 1.4784068 | 1.2896696 | 0.9924999 |
| 5299 | 935_at | CAP | 1 | Lower | 0.98 | 1.5250124 | 1.2581625 | 0.9758878 |

TABLE 13
Prognosis Genes for Longer (≧106 days) versus Shorter (<106 days) TTP

| SEQ ID NO: | Qualifier | Gene Name | Rank # | Class | Score | Perm 1% | Perm 5% | Perm (user) |
|---|---|---|---|---|---|---|---|---|
| 379 | 1653_at | RPS3A | 12 | Longer | 0.67 | 0.8055016 | 0.7561978 | 0.6425947 |
| | 1665_s_at | ECGF1 | 1 | Shorter | 0.85 | 1.0884173 | 1.014112 | 0.8190228 |
| 698 | 1815_g_at | TGFBR2 | 9 | Longer | 0.7 | 0.9029855 | 0.8274894 | 0.6774455 |
| 5165 | 31675_s_at | PTENP1 | 2 | Shorter | 0.85 | 0.98265 | 0.8774547 | 0.7430871 |
| 643 | 31993_f_at | UNK_U80764 | 7 | Longer | 0.77 | 1.0337092 | 0.970009 | 0.7476342 |
| 989 | 32569_at | PAFAH1B1 | 11 | Longer | 0.7 | 0.8284972 | 0.7577868 | 0.647478 |
| 200 | 33660_at | RPL5 | 10 | Longer | 0.7 | 0.8362634 | 0.782186 | 0.6625283 |
| 5220 | 37148_at | LILRB3 | 4 | Shorter | 0.77 | 0.9059746 | 0.8105006 | 0.6940544 |
| 1835 | 37343_at | ITPR3 | 8 | Longer | 0.76 | 0.9370008 | 0.8503211 | 0.7099578 |
| 4843 | 38397_at | UNK_U09196 | 3 | Shorter | 0.84 | 0.961974 | 0.841938 | 0.710196 |
| 4808 | 40607_at | DPYSL2 | 5 | Shorter | 0.75 | 0.8795726 | 0.7939292 | 0.6816332 |
| 5187 | 41045_at | SECTM1 | 6 | Shorter | 0.74 | 0.8546471 | 0.791536 | 0.6672204 |

Leave-one-out cross validation using the above-described gene classifiers for the clinical stratifications of intermediate versus poor prognosis Motzer risk, early progressors (TTP<106 days) versus all other patients, lower quartile TTP versus upper quartile TTP, and short term (survival<150 days) versus long term survivors (survival>550 days) yielded 74.4%, 77.8%, 77.3% and 79% overall accuracy for class assignment, respectively. Performance characteristics of the above-described classifiers are summarized in Table 14. The accuracy, sensitivity, and specificity for class assignment under each classifier using leave-one-out cross validation are demonstrated in the table. The k-nearest-neighbors algorithm as described in Armstrong, et al., supra, was employed for all evaluations.

TABLE 14
Performance Characteristics of Gene Classifiers from
Supervised Approaches

| | Size of | | | |
|---|---|---|---|---|
| | Optimal | | | |
| | Gene | Accuracy | Sensitivity | Specificity |
| Classification | Classifier | (%) | (%) | (%) |
| Motzer risk Poor vs | 42 | 74.4 | 72.7 | 76.5 |
| Intermediate | | | | |
| Progressive disease vs | 18 | 66.7 | 22.2 | 78.7 |
| any clinical response | | | | |
| Lowest quartile | 52 | 63.6 | 54.5 | 72.7 |
| survival vs | | | | |
| highest quartile | | | | |
| survival | | | | |
| Lowest quartile TTP vs | 6 | 77.3 | 81.8 | 72.7 |
| highest quartile TTP | | | | |
| Short term survival | 58 | 79.0 | 57.4 | 85.7 |
| (TTD < 150 days) | | | | |
| vs long term survival | | | | |
| (survival >550 days) | | | | |
| Early progression | 12 | 77.8 | 45.5 | 88.2 |
| TTP < 106 days | | | | |
| vs all other patients | | | | |

“Sensitivity” as used herein refers to the ratio of correct positive calls over the total of true positive calls plus false negative calls. “Specificity” refers to the ratio of correct negative calls over the total of true negative calls plus false positive calls. The genes identified in FIGS. 1A and 2A-2E and Tables 10-13, or the classifiers derived therefrom, can be used to assign an RCC patient to a respective clinical class selected from Table 14.

In yet another approach, unsupervised clustering was employed to identify genes that are correlated with survival. One of the primary endpoints of a clinical trial or a therapeutic treatment is survival. The above-described gene classifiers do not predict short term survival with supreme sensitivity and specificity (such as over 90%, 95%, or more). This might be due to heterogeneity in PBMC expression patterns from patients binned arbitrarily into different survival categories that precludes highly accurate prediction using forced-type supervised approaches. A pharmacogenomic assay capable of identifying short-term and long-term survivors in a significant fraction of the intended treatment population would still have obvious benefit, in terms of clinical prognosis. In an attempt to identify a more limited subset of patients with similar clinical outcomes for which class assignment would be more robust, an unsupervised hierarchical clustering approach using all genes passing the initial criteria (5,424 genes total) was employed.

The unsupervised hierarchical clustering was performed according to the procedure described in Eisen, et al., PROC NATL ACAD SCI U.S.A., 95:14863-14868 (1998). For hierarchical clustering, data were log transformed and normalized to have a mean value of zero and a variance of one. Hierarchical clustering results were generated using average linkage clustering and an uncentered correlation similarity metric.

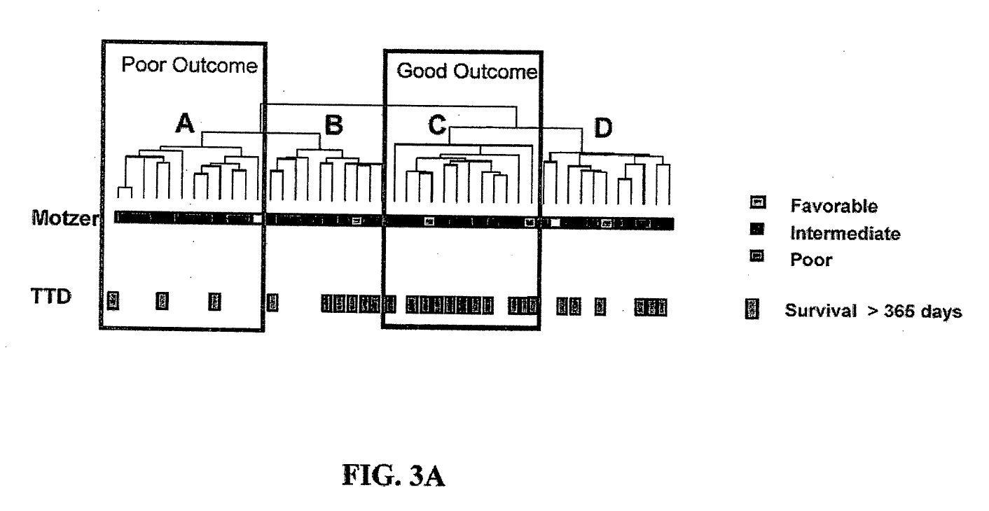

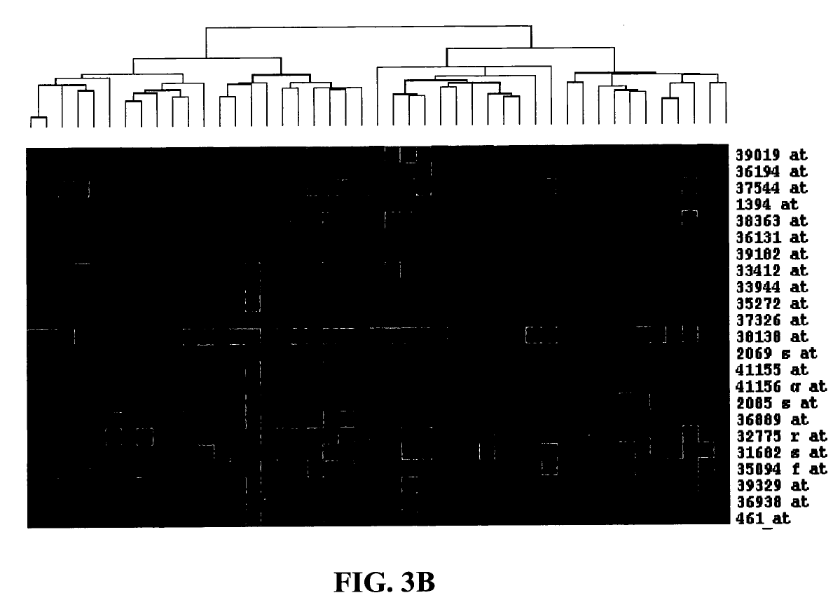

The dendrogram in FIG. 3A shows that sample relationships grouped the RCC PBMCs (n=45) into four roughly equivalent sized subclusters designated A through D. The majority of patients in cluster A possessed significantly shorter survival than the majority of patients in cluster C, suggesting that expression differences in these two subclusters of patients could be predictive of survival in the majority of patients in these subpopulations. RCC patient PBMC expression profiles in the poor prognosis cluster (“A”) are indicated by the box around subcluster “A” in which 9 out of 12 patients exhibited survival of less than 365 days. RCC patient PBMC expression profiles in the good prognosis cluster (“C”) are indicated by the box around subcluster “C” in which 10 out of 12 patients exhibited survival of 365 or more days. In addition, prognostic Motzer scores were distinct between subclusters A and C, as indicated in FIG. 3A.

FIG. 3B shows the baseline expression patterns of a group of selected genes in subclusters A-D. Elevated or decreased expression values relative to the average expression value across all experiments are indicated according to the scale of FIG. 1A.

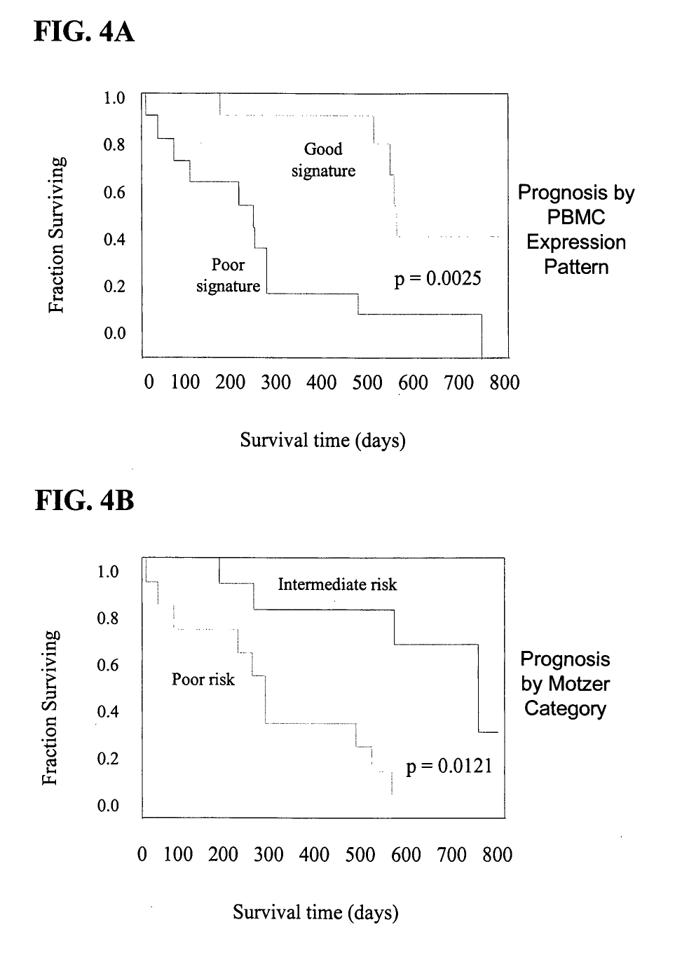

Kaplan-Meier analysis demonstrated that patients in the four subclusters possessed significant differences in survival (p=0.021, Wilcoxon test). Kaplan-Meier analysis showed that prognosis by PBMC gene expression signature in subgroups A (“Poor signature”) and C (“Good Signature”) yielded more significant differences in survival (p=0.0025, Wilcoxon test) than prognosis by the Motzer risk assessment (p=0.0125, Wilcoxon test). See FIG. 4A and FIG. 4B.

The above finding suggests that there exist biologically distinct differences in expression patterns of PBMCs that are predictive of survival in patients with RCC. Because it was possible that the observed differences in expression were driven by differences in patient demographics or even by technical differences in the samples, technical and demographical characteristics between these two subclusters (cluster “A” versus cluster “C”) were compared in Table 15 Comparison of technical and demographic parameters indicated no significant difference between these subgroups of patients, and the only significant differences between these groups appear to be the prognostic Motzer risk classification and the primary clinical endpoint of survival. Values for the individual parameters associated with profiles in each of the clusters were tested for differences (p-value).

TABLE 15
Significance Testing of Technical, Demographic, Prognostic and
Clinical Parameters Observed in Patients and PBMC profiles in Good
versus Poor Prognosis Clusters

| | | | |
|---|---|---|---|
| | Poor Prognosis | Good Prognosis | |
| Parameter | (Cluster “A”) | (Cluster “C”) | p-value |

Technical

| | | | |
|---|---|---|---|
| Raw Q | 2.34 | 2.45 | 0.5200 |
| GAPDH 5′/3′ ratio | 0.95 | 0.93 | 0.6600 |
| Scale factor | 2.94 | 2.69 | 0.5800 |
| Average frequency | 16.8 | 19.6 | 0.2000 |
| (ppm) | | | |
| Present calls | 4178 | 4194 | 0.9400 |

Demographical

| | | | |
|---|---|---|---|
| Sex | 9 male/3 female | 9 male/3 female | 1.000 |
| Age (years) | 59.3 | 53.8 | 0.0870 |
| Ethnicity | 100% Caucasian | 100% Caucasian | 1.000 |

Prognostic
assessment

| | | | |
|---|---|---|---|
| Motzer | 8 poor, 4 | 3 poor, 7 | N/A |
| classification | intermediate | intermediate, 2 | |
| | | favorable | |

Clinical endpoint

| | | | |
|---|---|---|---|
| Median survival | 281 | 573 | 0.0025 |
| time (days) | | | |
| Average TTP (days) | 117 | 240 | 0.1812^{b} |

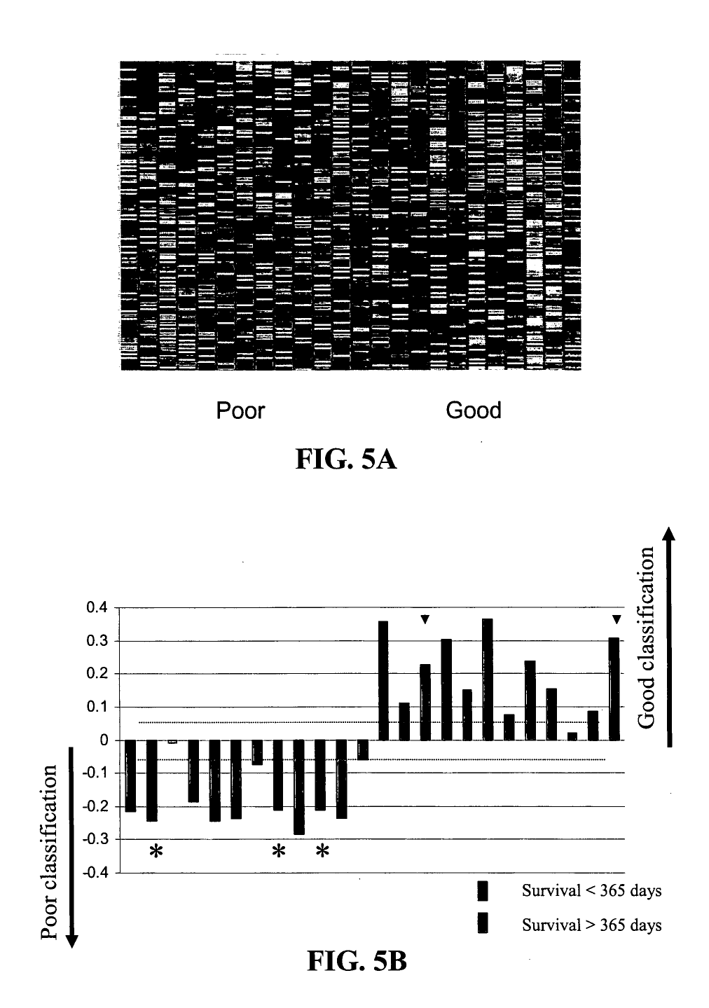

Given the robust differences in median survival times between PBMC profiles in the poor and good prognosis clusters, a nearest-neighbor algorithm was employed to identify the transcripts in the subsets of PBMCs that are significantly correlated with good and poor prognosis signatures. The relative expression levels of an optimally-sized gene classifier derived from this analysis are shown in FIG. 5A. The gene classifier was composed of 158 genes. Because the good prognosis and poor prognosis clusters were identified based upon their differences in gene expression, random permutation of this nearest-neighbor analyses showed the genes in the classifier to be significantly correlated as expected (p<0.01). The relative expression levels of each gene (rows) are indicated for each patient (columns) according to the scale depicted in FIG. 1A. Each gene in the classifier and its respective expression level in each class (poor versus good prognosis cluster) are summarized in Table 16.

TABLE 16
Prognosis Genes for Assigning Class Membership to Patients
in the Good and Poor Prognosis Subclusters

| SEQ ID NO: | Qualifier | Gene Name | Rank # | Class | Score | Perm 1% | Perm 5% | Perm (user) |
|---|---|---|---|---|---|---|---|---|
| 306 | 1034_at | TIMP3 | 90 | Good | 1.57 | 1.0445594 | 0.9665145 | 0.7096911 |
| 2737 | 1097_s_at | CCR7 | 155 | Good | 1.23 | 0.8934941 | 0.7093403 | 0.5209759 |
| 4924 | 1158_s_at | CALM3 | 71 | Poor | 0.98 | 1.0384812 | 0.7927625 | 0.5121112 |
| 1485 | 1267_at | PRKCH | 104 | Good | 1.46 | 0.9849667 | 0.8875619 | 0.6371682 |
| 5288 | 1315_at | UNK_D78361 | 11 | Poor | 1.51 | 1.1908811 | 0.9882026 | 0.6823562 |
| 5152 | 1323_at | UNK_X04803 | 76 | Poor | 0.96 | 1.0239922 | 0.7720025 | 0.5026828 |
| 5002 | 1424_s_at | YWHAH | 8 | Poor | 1.56 | 1.2260145 | 0.9882028 | 0.712902 |
| 2253 | 1479_g_at | ITK | 158 | Good | 1.22 | 0.8877654 | 0.7093402 | 0.5143056 |
| 987 | 1717_s_at | API2 | 85 | Good | 1.68 | 1.1154871 | 1.0003265 | 0.7644543 |
| 1179 | 202_at | HSF2 | 103 | Good | 1.5 | 0.9849667 | 0.900169 | 0.6398714 |
| 5124 | 2069_s_at | CTNNA1 | 9 | Poor | 1.55 | 1.205555 | 0.9882026 | 0.7047761 |
| 4681 | 2085_s_at | CTNNA1 | 40 | Poor | 1.16 | 1.1177368 | 0.8824167 | 0.5698908 |
| 4231 | 2090_i_at | UNK_H12458 | 62 | Poor | 1.01 | 1.0607328 | 0.8190967 | 0.525894 |
| 4988 | 268_at | PECAM1 | 34 | Poor | 1.25 | 1.1177368 | 0.9106545 | 0.5847529 |
| 5064 | 283_at | UQCRC1 | 27 | Poor | 1.32 | 1.1177368 | 0.9440462 | 0.6078221 |
| 5303 | 286_at | H2AFO | 55 | Poor | 1.06 | 1.07645 | 0.8355755 | 0.534318 |
| 4239 | 307_at | ALOX5 | 75 | Poor | 0.96 | 1.0283809 | 0.7769105 | 0.506168 |
| 5285 | 31444_s_at | ANXA2P2 | 2 | Poor | 1.67 | 1.3424762 | 1.1425713 | 0.8610321 |
| 5261 | 31504_at | HDLBP | 54 | Poor | 1.07 | 1.0793227 | 0.8362562 | 0.5385964 |
| 4398 | 31682_s_at | CSPG2 | 20 | Poor | 1.35 | 1.1213673 | 0.9803211 | 0.6337798 |
| 1232 | 32087_at | HSF2 | 146 | Good | 1.26 | 0.9003578 | 0.7252433 | 0.5342414 |
| 2181 | 32097_at | PCNT | 107 | Good | 1.44 | 0.9849666 | 0.8821047 | 0.6232104 |
| 5083 | 32153_s_at | UNK_U49869 | 46 | Poor | 1.13 | 1.1102691 | 0.8593918 | 0.5556471 |
| 603 | 32183_at | SFRS11 | 108 | Good | 1.43 | 0.9849666 | 0.8821047 | 0.6206105 |
| 277 | 32541_at | PPP3CC | 93 | Good | 1.56 | 1.0293367 | 0.9353749 | 0.6922912 |
| 393 | 32680_at | KIAA0551 | 157 | Good | 1.22 | 0.8893883 | 0.7093402 | 0.5186095 |
| 5270 | 32749_s_at | FLNA | 61 | Poor | 1.02 | 1.0607328 | 0.8224345 | 0.5291181 |
| 3956 | 32775_r_at | PLSCR1 | 78 | Poor | 0.95 | 1.0217315 | 0.7712451 | 0.4984867 |
| 5009 | 32800_at | RXRA | 79 | Poor | 0.95 | 1.0212312 | 0.7709695 | 0.4976646 |
| 638 | 32804_at | UNK_AF091263 | 142 | Good | 1.27 | 0.9081621 | 0.7369459 | 0.5426338 |
| 5004 | 32806_at | BZRP | 53 | Poor | 1.08 | 1.0906383 | 0.8376178 | 0.5398285 |
| 1331 | 33134_at | ADCY3 | 101 | Good | 1.52 | 1.0293367 | 0.9001691 | 0.6478866 |
| 1061 | 33267_at | UNK_AF035315 | 140 | Good | 1.28 | 0.9149016 | 0.7390382 | 0.5443108 |
| 5041 | 33371_s_at | RAB31 | 31 | Poor | 1.28 | 1.1177368 | 0.9281045 | 0.5929822 |
| 1715 | 33521_at | ATP4A | 111 | Good | 1.42 | 0.9608656 | 0.8507274 | 0.6137387 |
| 5300 | 33659_at | CFL1 | 77 | Poor | 0.96 | 1.0239921 | 0.7719817 | 0.5007594 |
| 4589 | 33733_at | ABCG2 | 67 | Poor | 0.99 | 1.054551 | 0.7991308 | 0.517656 |
| 5007 | 33777_at | TBXAS1 | 50 | Poor | 1.11 | 1.0997332 | 0.843028 | 0.5445145 |
| 895 | 33788_at | LAP70 | 94 | Good | 1.55 | 1.0293367 | 0.9353749 | 0.685765 |
| 1040 | 33797_at | MPHOSPH10 | 127 | Good | 1.33 | 0.9353749 | 0.7733641 | 0.571226 |
| 1231 | 33819_at | LDHB | 97 | Good | 1.54 | 1.0293367 | 0.9353749 | 0.674343 |
| 374 | 33847_s_at | UNK_AI304854 | 125 | Good | 1.34 | 0.9353749 | 0.804844 | 0.5768012 |
| 5262 | 33956_at | MD-2 | 3 | Poor | 1.62 | 1.2851958 | 1.1000433 | 0.8143725 |
| 4887 | 34033_s_at | LILRA2 | 49 | Poor | 1.11 | 1.1055416 | 0.8472178 | 0.5492646 |
| 223 | 34256_at | SIAT9 | 133 | Good | 1.31 | 0.9240009 | 0.7478784 | 0.5555079 |
| 4729 | 34268_at | GAIP | 52 | Poor | 1.1 | 1.0969372 | 0.840719 | 0.5409537 |
| 4709 | 34311_at | GLRX | 66 | Poor | 0.99 | 1.0546845 | 0.8039182 | 0.52042 |
| 5195 | 34400_at | QP-C | 72 | Poor | 0.98 | 1.0359778 | 0.7868937 | 0.5083646 |
| 1035 | 34654_at | MTMR1 | 100 | Good | 1.53 | 1.0293367 | 0.9353749 | 0.6529696 |
| 4652 | 34660_at | RNASE6 | 29 | Poor | 1.3 | 1.1177368 | 0.9303353 | 0.5994623 |
| 3748 | 34665_g_at | FCGR2B | 4 | Poor | 1.6 | 1.2845235 | 1.0609695 | 0.7795188 |
| 4579 | 34768_at | DKFZP564E1962 | 26 | Poor | 1.32 | 1.1177368 | 0.9440462 | 0.6101461 |
| 5199 | 34787_at | NRD1 | 37 | Poor | 1.18 | 1.1177368 | 0.8910962 | 0.5777738 |
| 303 | 34829_at | DKC1 | 115 | Good | 1.41 | 0.9353749 | 0.8407055 | 0.601603 |
| 1805 | 34983_at | CYP26A1 | 147 | Good | 1.26 | 0.9001691 | 0.7248857 | 0.531943 |
| 763 | 35238_at | TRAF5 | 92 | Good | 1.56 | 1.0293367 | 0.9353749 | 0.6963615 |
| 94 | 35286_r_at | RY1 | 135 | Good | 1.29 | 0.9199694 | 0.7444252 | 0.5536141 |
| 27 | 35319_at | CTCF | 89 | Good | 1.61 | 1.047387 | 0.9665145 | 0.7147587 |
| 194 | 35748_at | EEF1B2 | 141 | Good | 1.28 | 0.9107076 | 0.7378222 | 0.5433901 |
| 39 | 35753_at | PRP8 | 80 | Good | 1.79 | 1.2117286 | 1.1793184 | 0.9261844 |
| 4316 | 35773_i_at | NDUFB7 | 38 | Poor | 1.17 | 1.1177368 | 0.8840246 | 0.574061 |
| 1275 | 35802_at | KIAA1014 | 114 | Good | 1.41 | 0.9515032 | 0.8442041 | 0.6024917 |
| 5230 | 35810_at | ARPC3 | 13 | Poor | 1.49 | 1.185541 | 0.9882026 | 0.677634 |
| 3081 | 35853_at | PRKCABP | 126 | Good | 1.34 | 0.9353749 | 0.7811259 | 0.5762339 |
| 4826 | 35869_at | MD-1 | 22 | Poor | 1.34 | 1.1197696 | 0.9543627 | 0.6252207 |
| 3136 | 36021_at | UNK_AL049409 | 131 | Good | 1.31 | 0.9256013 | 0.7528632 | 0.560786 |
| 2558 | 36094_at | TNNT3 | 154 | Good | 1.23 | 0.8941191 | 0.7129431 | 0.5212274 |
| 5181 | 36130_f_at | MT1E | 69 | Poor | 0.98 | 1.0426595 | 0.7957169 | 0.5151819 |
| 682 | 36155_at | KIAA0275 | 134 | Good | 1.31 | 0.9219777 | 0.7474341 | 0.5539118 |
| 5272 | 36199_at | DAP | 32 | Poor | 1.27 | 1.1177368 | 0.9266225 | 0.5910927 |
| 771 | 36231_at | UNK_AC002073 | 102 | Good | 1.51 | 0.9849667 | 0.900169 | 0.6418348 |
| 1253 | 36403_s_at | UNK_AI434146 | 87 | Good | 1.65 | 1.0968254 | 0.9748203 | 0.735724 |
| 49 | 36456_at | DKFZP4564I052 | 105 | Good | 1.45 | 0.9849667 | 0.8821048 | 0.6290045 |
| 4836 | 36488_at | EGFL5 | 45 | Poor | 1.14 | 1.1102691 | 0.8690057 | 0.5559479 |
| 2214 | 36545_s_at | UNK_AB011114 | 153 | Good | 1.24 | 0.894684 | 0.7135342 | 0.5253994 |
| 5264 | 36675_r_at | PFN1 | 43 | Poor | 1.16 | 1.1110159 | 0.8790239 | 0.5610438 |
| 5268 | 36753_at | LILRB4 | 64 | Poor | 1 | 1.0607327 | 0.8089606 | 0.5225678 |
| 4885 | 36780_at | CLU | 41 | Poor | 1.16 | 1.1177368 | 0.8801252 | 0.5655208 |
| 446 | 36786_at | UNK_AL022721 | 148 | Good | 1.25 | 0.9001691 | 0.7246513 | 0.5306757 |
| 4926 | 36889_at | FCER1G | 16 | Poor | 1.4 | 1.1736697 | 0.9882026 | 0.6554383 |
| 136 | 36949_at | CSNK1D | 128 | Good | 1.33 | 0.9343908 | 0.7698004 | 0.5710414 |
| 5284 | 36963_at | PGD | 63 | Poor | 1 | 1.0607327 | 0.8144836 | 0.5233427 |
| 652 | 37005_at | NBL1 | 123 | Good | 1.35 | 0.9353749 | 0.8079842 | 0.58034 |
| 5053 | 37021_at | CTSH | 12 | Poor | 1.5 | 1.1857843 | 0.9882026 | 0.6823562 |
| 820 | 37078_at | CD3Z | 109 | Good | 1.43 | 0.9660364 | 0.8821047 | 0.6199971 |
| 5220 | 37148_at | LILRB3 | 5 | Poor | 1.59 | 1.2723099 | 1.0463293 | 0.766221 |
| 5116 | 37220_at | FCGR1A | 6 | Poor | 1.58 | 1.2682304 | 1.0439228 | 0.7437066 |
| 5185 | 37311_at | TALDO1 | 24 | Poor | 1.33 | 1.1197696 | 0.9471594 | 0.6192882 |
| 1835 | 37343_at | ITPR3 | 143 | Good | 1.27 | 0.908162 | 0.7334376 | 0.5423645 |
| 2386 | 37462_i_at | SF3A2 | 144 | Good | 1.27 | 0.9061325 | 0.732057 | 0.5407514 |
| 4694 | 37647_at | AOAH | 48 | Poor | 1.11 | 1.1081125 | 0.8492031 | 0.5507001 |
| 3961 | 37689_s_at | FCGR2A | 33 | Poor | 1.26 | 1.1177368 | 0.9106545 | 0.5880215 |
| 714 | 37727_i_at | RCN2 | 86 | Good | 1.67 | 1.1081127 | 0.994302 | 0.7482241 |
| 578 | 38019_at | CSNK1E | 95 | Good | 1.55 | 1.0293367 | 0.9353749 | 0.6819632 |
| 1604 | 38030_at | KIAA0332 | 124 | Good | 1.34 | 0.9353749 | 0.8055808 | 0.5792956 |
| 5085 | 38081_at | LTA4H | 10 | Poor | 1.54 | 1.1932396 | 0.9882026 | 0.6909306 |
| 5010 | 38111_at | CSPG2 | 21 | Poor | 1.34 | 1.1197697 | 0.9688478 | 0.6295477 |
| 4828 | 38112_g_at | CSPG2 | 14 | Poor | 1.46 | 1.180172 | 0.9882026 | 0.6708109 |
| 788 | 38113_at | KIAA0796 | 145 | Good | 1.27 | 0.9005588 | 0.7278896 | 0.5385186 |
| 690 | 38148_at | CRY1 | 112 | Good | 1.42 | 0.9515032 | 0.8507274 | 0.6115539 |
| 5186 | 38363_at | TYROBP | 23 | Poor | 1.34 | 1.1197696 | 0.9526655 | 0.6241006 |
| 4984 | 384_at | PSMB10 | 65 | Poor | 0.99 | 1.0564462 | 0.8055046 | 0.521621 |
| 5312 | 38483_at | HSA011916 | 47 | Poor | 1.12 | 1.1081127 | 0.8492386 | 0.5539569 |
| 81 | 38527_at | NONO | 139 | Good | 1.28 | 0.9153488 | 0.7403588 | 0.5454356 |
| 482 | 38542_at | NPM1 | 152 | Good | 1.24 | 0.8994522 | 0.7153279 | 0.5264516 |
| 4791 | 38621_at | UNK_AJ012008 | 70 | Poor | 0.98 | 1.040839 | 0.7946492 | 0.5128855 |
| 11 | 38702_at | UNK_AF070640 | 113 | Good | 1.42 | 0.9515032 | 0.8507274 | 0.6056142 |
| 886 | 38843_at | HMG2L1 | 110 | Good | 1.42 | 0.9609948 | 0.851209 | 0.6164021 |
| 5192 | 39043_at | ARPC1B | 42 | Poor | 1.16 | 1.1148714 | 0.8795826 | 0.5652992 |
| 189 | 39047_at | KIAA0156 | 121 | Good | 1.36 | 0.9353749 | 0.8101286 | 0.586782 |
| 4357 | 39320_at | CASP1 | 51 | Poor | 1.1 | 1.0991173 | 0.842422 | 0.5426413 |
| 5169 | 39329_at | ACTN1 | 57 | Poor | 1.06 | 1.0681722 | 0.829216 | 0.5313845 |
| 5240 | 39347_at | CLAPS2 | 39 | Poor | 1.17 | 1.1177368 | 0.8826484 | 0.5715545 |
| 5161 | 39360_at | SNX3 | 19 | Poor | 1.35 | 1.1216959 | 0.9803817 | 0.6441603 |
| 450 | 39509_at | UNK_AI692348 | 129 | Good | 1.33 | 0.9292568 | 0.7677588 | 0.5698953 |
| 48 | 39727_at | DUSP11 | 136 | Good | 1.29 | 0.9195961 | 0.7429254 | 0.5511671 |
| 5157 | 39749_at | PSMD4 | 18 | Poor | 1.36 | 1.1250532 | 0.9814645 | 0.6473147 |
| 69 | 39864_at | CIRBP | 106 | Good | 1.44 | 0.9849667 | 0.8821048 | 0.6261963 |
| 4745 | 39971_at | LYL1 | 73 | Poor | 0.97 | 1.034686 | 0.782038 | 0.5071201 |
| 4886 | 39997_at | PFC | 58 | Poor | 1.05 | 1.0657651 | 0.8253264 | 0.530821 |
| 2159 | 40016_g_at | KIAA0303 | 151 | Good | 1.24 | 0.9001691 | 0.7179127 | 0.5294558 |
| 808 | 40048_at | UNK_D43951 | 130 | Good | 1.31 | 0.9281045 | 0.7539371 | 0.5626611 |
| 1261 | 40092_at | BAZ2A | 132 | Good | 1.31 | 0.9250832 | 0.7514259 | 0.558431 |
| 121 | 40219_at | HIS1 | 138 | Good | 1.28 | 0.9159876 | 0.741164 | 0.5472672 |
| 151 | 40308_at | UNK_AI830496 | 82 | Good | 1.72 | 1.1483467 | 1.0505675 | 0.8307809 |
| 5196 | 40432_at | UNK_AA522891 | 35 | Poor | 1.2 | 1.1177368 | 0.8967329 | 0.5814439 |
| 521 | 40442_f_at | DKFZP564M2423 | 98 | Good | 1.54 | 1.0293367 | 0.9353749 | 0.6680597 |
| 1632 | 40511_at | GATA3 | 118 | Good | 1.39 | 0.9353749 | 0.839215 | 0.5919577 |
| 4808 | 40607_at | DPYSL2 | 59 | Poor | 1.02 | 1.0607328 | 0.8238136 | 0.5300378 |
| 2130 | 40667_at | CD6 | 96 | Good | 1.54 | 1.0293367 | 0.9353749 | 0.6789862 |
| 1787 | 40775_at | ITM2A | 149 | Good | 1.25 | 0.9001691 | 0.7227228 | 0.530193 |
| 152 | 40803_at | UNK_AL050161 | 150 | Good | 1.25 | 0.9001691 | 0.7224947 | 0.5295897 |
| 279 | 40868_at | UNK_AA442799 | 120 | Good | 1.38 | 0.9353749 | 0.8153356 | 0.5874232 |
| 1608 | 40896_at | POU2F1 | 156 | Good | 1.23 | 0.8902482 | 0.7093403 | 0.518687 |
| 5187 | 41045_at | SECTM1 | 30 | Poor | 1.28 | 1.1177368 | 0.9303353 | 0.5970426 |
| 5244 | 41136_s_at | APP | 68 | Poor | 0.99 | 1.0522225 | 0.7964097 | 0.5154617 |
| 4972 | 41153_f_at | CTNNA1 | 7 | Poor | 1.57 | 1.2448796 | 1.0065852 | 0.7184531 |
| 4911 | 41155_at | CTNNA1 | 17 | Poor | 1.38 | 1.1483397 | 0.986167 | 0.6532167 |
| 5111 | 41156_g_at | CTNNA1 | 15 | Poor | 1.42 | 1.1749569 | 0.9882026 | 0.6594592 |
| 495 | 41224_at | KIAA0788 | 88 | Good | 1.62 | 1.065765 | 0.9665145 | 0.7224365 |
| 146 | 41256_at | EEF1D | 122 | Good | 1.35 | 0.9353749 | 0.8079842 | 0.5821422 |
| 51 | 41288_at | CALM1 | 119 | Good | 1.39 | 0.9353749 | 0.8157417 | 0.5898756 |
| 4145 | 41300_s_at | ITM2B | 56 | Poor | 1.06 | 1.0734221 | 0.8317348 | 0.5335996 |
| 274 | 41337_at | AES | 117 | Good | 1.4 | 0.9353749 | 0.8396499 | 0.5959157 |
| 724 | 41338_at | AES | 83 | Good | 1.71 | 1.1325878 | 1.0314 | 0.8103616 |
| 182 | 41569_at | KIAA0974 | 99 | Good | 1.53 | 1.0293367 | 0.9353749 | 0.6633855 |
| 1748 | 41577_at | KIAA0823 | 91 | Good | 1.57 | 1.0398163 | 0.9665145 | 0.7029273 |
| 755 | 41669_at | KIAA0191 | 116 | Good | 1.41 | 0.9353749 | 0.8399643 | 0.5969042 |
| 5167 | 41745_at | IFITM3 | 74 | Poor | 0.97 | 1.0346859 | 0.7782155 | 0.5068782 |
| 5175 | 430_at | NP | 25 | Poor | 1.33 | 1.1177368 | 0.9440463 | 0.6164243 |
| 4686 | 574_s_at | CASP1 | 36 | Poor | 1.2 | 1.1177368 | 0.8938736 | 0.5782988 |
| 1283 | 663_at | EIF1A | 137 | Good | 1.29 | 0.9186698 | 0.741367 | 0.5490503 |
| 5281 | 769_s_at | ANXA2 | 1 | Poor | 1.77 | 1.4823041 | 1.2688332 | 0.9412579 |
| 4412 | 777_at | GDI2 | 44 | Poor | 1.15 | 1.1102691 | 0.8734871 | 0.5567811 |
| 919 | 840_at | ZNF220 | 81 | Good | 1.77 | 1.1495084 | 1.0703588 | 0.8762291 |
| 5207 | 880_at | FKBP1A | 28 | Poor | 1.31 | 1.1177368 | 0.9303353 | 0.6 |
| 1899 | 906_at | STAT4 | 84 | Good | 1.7 | 1.118592 | 1.0010654 | 0.7911333 |
| | AFFX- | BACTINM_Hs_AFFX | 60 | Poor | 1.02 | 1.0607328 | 0.8230627 | 0.5292476 |
| | HSAC07/X00351_M_at | | | | | | | |

Leave-one-out cross validation using the 158-gene classifier for predicting good versus poor prognosis gene signature yielded 100% overall accuracy for class assignment. However, three of the patients in the poor prognosis cluster actually possessed substantially longer survival times, and two of the patients whose PBMC profiles segregated with the good prognosis cluster actually possessed shorter survival times. To estimate the accuracy, sensitivity and specificity of this gene classifier with respect to true clinical outcome, a poor outcome was arbitrarily defined as <365 days survival and a good outcome was defined as ≧365 days. We took into account the incorrect assignment of the outlier profiles in the clusters and defined the objective of the clinical assay as the identification of patients with short (less than 1 year) survival times. Using these criteria the performance of the 158-gene classifier (by leave-one-out cross validation) demonstrated 79% overall accuracy, correctly identifying 9 of 11 patients with short survival times (less than 1 year, 82% sensitivity) and 10 of 13 patients with long term survival times (greater than 1 year, 77% specificity). See FIG. 5B. In FIG. 5B, the confidence scores were calculated for each sample in the analysis. For the purposes of illustration, prediction strengths accompanying calls of “survival≧1 year” were assigned positive values, and prediction strengths accompanying calls of “survival<1 year” were assigned negative values. Asterisks identify the false positives in this clinical assay designed to identify short survival times, and arrowheads indicate false negatives.

As appreciated by one of ordinary skill in the art, prognosis genes for other solid tumors can be similarly identified according to the present invention. These genes are differentially expressed in peripheral blood cells of solid tumor patients having different clinical outcomes.

III. Prognosis and Selection of Treatment of RCC and Other Solid Tumors

The prognosis genes of the present invention can be used as surrogate markers for the prognosis of solid tumors. The prognosis genes of the present invention can also be used to select optimal treatments of solid tumors. For instance, clinical outcomes of different treatments for a solid tumor can be analyzed by using peripheral blood expression profiling. Treatments with favorable prognoses are selected for patients of interest.

Any solid tumor, treatment, or clinical outcome can be assessed by the present invention. As described above, clinical outcome can be measured by TTP (e.g., less than or greater than a specified period), TTD (e.g., less than or greater than a specified period), progressive disease, non-progressive disease, stable disease, complete response, partial response, minor response, or a combination thereof. Clinical outcome can also be prognosticated based on clinical classifications under traditional risk assessment methods (such as Motzer risk assessment for RCC, as described in Motzer, et al., supra). In addition, non-responsiveness to a therapeutic treatment is also considered a measurable outcome.

To predict clinical outcome of a patient of interest, the peripheral blood expression profile of one or more prognosis genes in the patient of interest is compared to at least one reference expression profile. Any number of prognosis genes can be used. In many embodiments, the PBMC expression profiles of the prognosis genes are correlated with patient outcome under a class-based correlation metric (such as nearest-neighbor analysis) or a statistical method (such as Spearman's rank correlation or Cox proportional hazard regression model). In one example, the prognosis genes are differentially expressed in PBMCs of one class of patients as compared to another class of patients. Both classes of patients have a solid tumor, and each class of patients has a different clinical outcome. In another example, the PBMC expression level of each prognosis gene is substantially higher or substantially lower in PBMCs of one class of patients than that in another class of patients. In still another example, the prognosis genes are substantially correlated with a class distinction between two classes of patients, where the two classes of patients have the same disease as the patient of interest, and each class of patients has a different clinical outcome. In many cases, the prognosis genes are correlated with the class distinction at above the 50%, 25%, 10%, 5%, or 1% significance level under random permutation tests.

One or more reference expression profiles can be used. The reference expression profile(s) can be determined concurrently with the expression profile of the patient of interest. The reference expression profile(s) can also be predetermined or prerecorded in an electronic or another storage medium. In one embodiment, the reference expression profile(s) is an average expression profile of the prognosis genes in peripheral blood samples of reference patients. Any averaging algorithm can be used to prepare the reference expression profile(s). In many cases, the reference patients have the same solid tumor as the patient of interest, and the clinical outcome of the reference patients is either known or determinable. In another embodiment, the reference patients can be divided into at least two classes, each class having a different respective clinical outcome. The peripheral blood expression profile of the prognosis genes in each class of the reference patients constitutes a separate reference profile.

The expression profile of the patient of interest and the reference expression profile(s) can be in any form. In one embodiment, the expression profiles comprise the expression level of each prognosis gene used in the comparison. The expression levels can have absolute, normalized, or relative values. Suitable normalization procedures include, but are not limited to, those used in nucleic acid array gene expression analyses or those described in Hill, et al., GENOME BIOL, 2:research0055.1-0055.13 (2001). In one example, the expression levels are normalized such that the mean is zero and the standard deviation is one. In another example, the expression levels are normalized based on internal or external controls, as appreciated by those skilled in the art. In still another example, the expression levels are normalized against one or more control transcripts with known abundances in blood samples. In many cases, the expression profile of the patient of interest and the reference expression profile(s) are constructed using the same or comparable methodology.

In another embodiment, the expression profiles comprise one or more ratios between the expression levels of different prognosis genes. The expression profiles can also include other measures that are capable of representing gene expression patterns.

The peripheral blood samples used in the present invention can be either whole blood samples, or samples comprising enriched PBMCs. In one example, the peripheral blood samples from the reference patients comprise enriched or purified PBMCs, and the peripheral blood sample from the patient of interest is a whole blood sample. In another example, all of the peripheral blood samples employed in the analysis comprise enriched or purified PBMCs. In many cases, the peripheral blood samples are prepared from the patient of interest and the reference patients by using the same or comparable procedures.

Other types of blood samples can also be employed in the present invention, provided that a statistically significant correlation exists between patient outcome and the gene expression profile in these blood samples.

The peripheral blood samples used in the present invention can be isolated from respective patients at any disease or treatment stage, provided that the correlation between the gene expression patterns in these peripheral blood samples and clinical outcome is statistically significant. In one embodiment, clinical outcome is measured by patients' response to a therapeutic treatment, and all of the blood samples used in the analysis are isolated prior to the therapeutic treatment. The expression profiles derived from these blood samples are baseline expression profiles for the therapeutic treatment.

Construction of the expression profiles typically involves detection of the expression level of each prognosis gene used in the comparison. Numerous methods are available for this purpose. For instance, the expression level of a gene can be determined by measuring the level of the RNA transcript(s) of the gene. Suitable methods include, but are not limited to, quantitative RT-PCR, Northern blot, in situ hybridization, slot-blotting, nuclease protection assay, and nucleic acid array (including bead array). The expression level of a gene can also be determined by measuring the level of the polypeptide(s) encoded by the gene. Suitable methods include, but are not limited to, immunoassays (such as ELISA, RIA, FACS, or Western blot), 2-dimensional gel electrophoresis, mass spectrometry, or protein arrays.

In one aspect, the expression level of a prognosis gene is determined by measuring the RNA transcript level of the gene in a peripheral blood sample. RNA can be isolated from the peripheral blood sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR (RT-PCR), isothermal amplification, ligase chain reaction, and Qbeta replicase.

In one embodiment, the amplification protocol employs reverse transcriptase. The isolated mRNA can be reverse transcribed into cDNA using a reverse transcriptase, and a primer consisting of oligo d(T) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TaqMan, ABI) is used for detecting or comparing the RNA transcript level of a prognosis gene of interest. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a prognosis gene of interest. The nucleic acid arrays can be commercial oligonucleotide or cDNA arrays. They can also be custom arrays comprising concentrated probes for the prognosis genes of the present invention. In many examples, at least 15%, 20%, 25%, 30%, 35%, 40%, 45%, 50%, or more of the total probes on a custom array of the present invention are probes for solid tumor prognosis genes. These probes can hybridize under stringent or nucleic acid array hybridization conditions to the RNA transcripts, or the complements thereof, of the corresponding prognosis genes.

As used herein, “stringent conditions” are at least as stringent as, for example, conditions G-L shown in Table 17. “Highly stringent conditions” are at least as stringent as conditions A-F shown in Table 17. As used in Table 1, hybridization is carried out under the hybridization conditions (Hybridization Temperature and Buffer) for about four hours, followed by two 20-minute washes under the corresponding wash conditions (Wash Temp. and Buffer).

TABLE 17
Stringency Conditions

| Stringency | Polynucleotide | Hybrid | Hybridization | Wash Temp. |
|---|---|---|---|---|
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1x SSPE is 0.15M NaCl, 10 mM NaH₂PO₄, and 1.25 mM EDTA, pH 7.4) can be substituted for SSC (1x SSC is 0.15M NaCl and 15 mM sodium citrate) in the hybridization and wash buffers. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81. | | | | |

In one example, a nucleic acid array of the present invention includes at least 2, 5, 10, 15, 20, 25, 30, 35, 40, 45, 50, 60, 70, 80, 90, 100, 150, 200, 250, or more different probes. Each of these probes is capable of hybridizing under stringent or nucleic acid array hybridization conditions to a different respective prognosis gene of the present invention. Multiple probes for the same prognosis gene can be used on the same nucleic acid array. The probe density on the array can be in any range. For instance, the density can be at least (or no more than) 5, 10, 25, 50, 100, 200, 300, 400, or 500, 1,000, 2,000, 3,000, 4,000, 5,000, or more probes/cm².

The probes can be DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the prognosis genes can be stably attached to discrete regions on the nucleic acid array. By “stably attached,” it means that a probe maintains its position relative to the attached discrete region during hybridization and signal detection. The position of each discrete region on the nucleic acid array can be either known or determinable. All of the methods known in the art can be used to make the nucleic acid arrays of the present invention.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

Hybridization probes or amplification primers for the prognosis genes of the present invention can be prepared by using any method known in the art. For prognosis genes whose genomic locations have not been determined or whose identities are solely based on EST or mRNA data, the probes/primers for these genes can be derived from the corresponding SEQ ID NOs, Entrez accession numbers, or EST or mRNA sequences.

In one embodiment, the probes/primers for each prognosis gene significantly diverge from the sequences of other prognosis genes. This can be achieved by checking potential probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always>0) and N (penalty score for mismatching residues; always<0). The BLAST algorithm parameters W, T, and X determine the sensitivity and speed of the alignment. These parameters can be adjusted for different purposes, as appreciated by those skilled in the art.

In another aspect, the expression levels of the prognosis genes of the present invention are determined by measuring the levels of polypeptides encoded by the prognosis genes. Methods suitable for this purpose include, but are not limited to, immunoassays such as ELISA, RIA, FACS, dot blot, Western Blot, immunohistochemistry, and antibody-based radioimaging. In addition, high-throughput protein sequencing, 2-dimensional SDS-polyacrylamide gel electrophoresis, mass spectrometry, or protein arrays can be used.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can be detected. Detection can be achieved by the addition of a second antibody which is specific for the target proteins and is linked to a detectable label. Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying ELISA, the samples suspected of containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing these antibodies are well known in the art. In one embodiment, the antibodies of the present invention can bind to the corresponding prognosis gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the prognosis genes. Methods for making protein arrays or biochips are well known in the art. In many embodiments, a substantial portion of probes on a protein array of the present invention are antibodies specific for the prognosis gene products. For instance, at least 10%, 20%, 30%, 40%, 50%, or more probes on the protein array can be antibodies specific for the prognosis gene products.

In yet another aspect, the expression levels of the prognosis genes of are determined by measuring the biological functions or activities of these genes. Where a biological function or activity of a gene is known, suitable in vitro or in vivo assays can be developed to evaluate the function or activity. These assays can be subsequently used to assess the level of expression of the prognosis gene.

With the expression level of each prognosis gene determined, numerous approaches can be employed to compare expression profiles. Comparison between the expression profile of a patient of interest and the reference expression profile(s) can be conducted manually or electronically. In one example, comparison is carried out by comparing each component in one expression to the corresponding component in another expression profile. The component can be the expression level of a prognosis gene, a ratio between the expression levels of two prognosis genes, or another measure capable of representing gene expression patterns. The expression level of a gene can have an absolute or a normalized or relative value. The difference between two corresponding components can be assessed by fold changes, absolute differences, or other suitable means.

Comparison between expression profiles can also be conducted using pattern recognition or comparison programs, such as the k-nearest-neighbors algorithm as described in Armstrong, et al., supra, or the weighted voting algorithm as described below. In addition, the serial analysis of gene expression (SAGE) technology, the GEMTOOLS gene expression analysis program (Incyte Pharmaceuticals), the GeneCalling and Quantitative Expression Analysis technology (Curagen), and other suitable methods, programs or systems can be used to compare expression profiles.

Multiple prognosis genes can be used in the comparison of expression profiles. For instance, 2, 4, 6, 8, 10, 12, 14, 16, 18, 20, 30, 40, 50, or more prognosis genes can be used. In addition, the prognosis gene(s) used in the comparison can be selected to have relatively small p-values (e.g., two-sided p-values). In one example, the p-values indicate the statistical significance of the difference between gene expression levels in different classes of patients. In another example, the p-values suggest the statistical significance of the correlation between gene expression patterns and clinical outcome. In one embodiment, the prognosis genes used in the comparison have p-values of no greater than 0.05, 0.01, 0.001, 0.0005, 0.0001, or less. Prognosis genes with p-values of greater than 0.05 can also be used. These genes may be identified, for instance, by using a relatively small number of blood samples.

Similarity or difference between the expression profile of a patient of interest and the reference expression profile(s) is indicative of the class membership of the patient of interest. Similarity or difference can be determined by any suitable means.

In one example, a component in a reference profile is a mean value, and the corresponding component in the expression profile of the patient of interest falls within the standard deviation of the mean value. In such a case, the expression profile of the patient of interest may be considered similar to the reference profile with respect to that particular component. Other criteria, such as a multiple or fraction of the standard deviation or a certain degree of percentage increase or decrease, can be used to measure similarity.

In another example, at least 50% (e.g., at least 60%, 70%, 80%, 90%, or more) of the components in the expression profile of the patient of interest are considered similar to the corresponding components in a reference profile. Under these circumstances, the expression profile of the patient of interest may be considered similar to the reference profile. Different components in the expression profile may have different weights for the comparison. In some cases, lower percentage thresholds (e.g., less than 50% of the total components) are used to determine similarity.

The prognosis gene(s) and the similarity criteria can be selected such that the accuracy of outcome prediction (the ratio of correct calls over the total of correct and incorrect calls) is relatively high. For instance, the accuracy of prediction can be at least 50%, 60%, 70%, 80%, 90%, or more. Prognosis genes with prediction accuracy of less than 50% can also be used, provided that the prediction is statistically significant.

The effectiveness of outcome prediction can also be assessed by sensitivity and specificity. The prognosis genes and the comparison criteria can be selected such that both the sensitivity and specificity of outcome prediction are relatively high. For instance, the sensitivity and specificity can be at least 50%, 60%, 70%, 80%, 90%, 95%, or more. Prognosis genes having lower sensitivity or specificity can be used as long as the prediction is statistically significant.

Moreover, gene expression-based outcome prediction can be combined with other clinical evidence or prognostic methods to improve the effectiveness or accuracy of outcome prediction.

In one embodiment, the expression profile of a patient of interest is compared to at least two reference expression profiles. The first reference expression profile can be prepared from peripheral blood samples of patients in a first outcome class, and the second reference expression profile is prepared from peripheral blood samples of patients in a second outcome class. The fact that the expression profile of the patient of interest is more similar to the first reference profile than to the second reference profile suggests that the patient of interest is more likely to belong to the first outcome class, as opposed to the second outcome class.

Comparison between the expression profile of a patient of interest and two or more reference expression profiles can be performed by any suitable means. In one embodiment, the k-nearest-neighbors algorithm, as described in Armstrong, et al., supra, is used. The k-nearest-neighbors algorithm can effectively assign a patient to a clinical class. By “effectively,” it means that the assignment is statistically significant. For instance, the sensitivity and specificity of the assignment can be at least 50%, 60%, 70%, 80%, 90%, 95%, or more. In one example, the effectiveness of assignment is evaluated based on leave-one-out cross validation. The accuracy for leave-one-out cross validation can be, for instance, at least 50%, 60%, 70%, 80%, 90%, 95%, or more. Prognosis genes or class predictors with low assignment sensitivity/specificity or leave-one-out cross validation accuracy, such as less than 50%, can also be used in the present invention.

In another embodiment, a weighted voting algorithm is used. In this method, the expression level of each gene in the classifier set contributes to an overall vote on the classification of the sample. See Slonim, et al., supra. The prediction strength is a combined variable that indicates the support for one class or the other, and can vary between 0 (narrow margin of victory) and 1 (wide margin of victory) in favor of the predicted class. See Golub, et al., supra, and Slonim, et al., supra. Software programs suitable for the weight voting analysis include, but are not limited to, GeneCluster 2 software. GeneCluster 2 software is available from MIT Center for Genome Research at Whitehead Institute (e.g., www-genome.wi.mit.edu/cancer/software/genecluster2/gc2.html).

Under one form of the weighted voting algorithm, a set of prognosis genes are selected to create a class predictor (classifier). Each gene in the class predictor casts a weighted vote for one of the two classes (class 0 and class 1). The vote of gene “g” can be defined as v_{g}=a_{g}(x_{g}−b_{g}), wherein a_{g} equals to P(g,c) and reflects the correlation between the expression level of gene “g” and the class distinction between the two classes, b_{g} is calculated as b_{g}=[x0(g)+x1(g)]/2 and represents the average of the mean logs of the expression levels of gene “g” in class 0 and class 1, and x_{g} is the normalized log of the expression level of gene “g” in the sample of interest. A positive v_{g} indicates a vote for class 0, and a negative v_{g} indicates a vote for class 1. V0 denotes the sum of all positive votes, and V1 denotes the absolute value of the sum of all negative votes. A prediction strength PS is defined as PS=(V0−V1)/(V0+V1).

Cross-validation can be used to evaluate the accuracy of the class predictor created under the k-nearest-neighbors or weighted voting algorithm. Briefly, one sample which has been used to identify the prognosis genes under the neighborhood analysis is withheld. A class predictor is then created based on the remaining samples and used to predict the class of the sample withheld. This process can be repeated for each sample that has been used in the neighborhood analysis. Different class predictors can be evaluated using the cross-validation process, and the best class predictor with the most accurate predication can be identified.

Suitable prediction strength (PS) thresholds can be assessed by plotting the cumulative cross-validation error rate against the prediction strength. In one embodiment, a positive predication is made if the absolute value of PS for the sample of interest is no less than 0.3. Other PS thresholds, such as no less than 0.1, 0.2, 0.4 or 0.5, can also be used. In many embodiments, a threshold is selected such as the accuracy of prediction is optimized and the incidence of both false positive and false negative results is minimized.

In one example, the class predictor includes n prognosis genes identified under the neighborhood analysis. A half of these prognosis genes has the largest P(g,c) scores, and the other half has the largest −P(g,c) scores. The number n therefore is the only free parameter in defining the class predictor.

The prognosis genes or class predictors of the present invention can be used to assign a solid tumor patient of interest to an outcome class. In one embodiment, patients having the solid tumor can be divided into at least two classes. The first class of patients has a first specified TTD (e.g., TTD of less than 150 days from initiation of a therapeutic treatment of the solid tumor), and the second class of patients has a second specified TTD (e.g., TTD of more than 550 days from initiation of the therapeutic treatment). Genes that are substantially correlated with the class distinction between these two classes of patients can be identified and used to assign the patient of interest to one of these two outcome classes. In one example, all of the expression profiles used in the comparison are baseline profiles which are prepared from baseline peripheral blood samples isolated prior to a therapeutic treatment. In another example, the solid tumor to be prognosed is RCC, and the therapeutic treatment is a CCI-779 therapy. The prognosis gene(s) used for outcome prediction can be selected from, for instance, Table 10.

In another embodiment, the first class of patients has a specified TTP (e.g., TTP of no less than 106 days from initiation of a therapeutic treatment), and the second class of patients has another specified TTP (e.g., TTP of less than 106 days from initiation of the therapeutic treatment). The solid tumor can be RCC, and the therapeutic treatment can be a CCI-779 therapy. The prognosis gene(s) can be selected from, for instance, Table 13.

In yet another embodiment, the first class of patients includes or consists of patients having the lowest quartile of TTP among a population of patients who have the same solid tumor and are subject to the same therapeutic treatment. The second class of patients includes or consists of patients having the highest quartile of TTP among the population of patients. The solid tumor can be RCC, and the therapeutic treatment can be a CCI-779 therapy. The prognosis gene(s) can be selected from, for instance, Table 12.

In still yet another embodiment, the first class of patients includes or consists of patients having the lowest quartile of TTD among a population of patients who have the same solid tumor and are subject to the same therapeutic treatment, and the second class of patients includes or consists of patients having the highest quartile of TTD among the population of patients. The solid tumor can be RCC, and the therapeutic treatment can be a CCI-779 therapy.

In a further embodiment, the first class of patients has a prognosis determined by a risk assessment method, and the second class of patients has another prognosis determined by the same risk assessment method. In one example, both classes of patients have RCC, and the risk assessment method is based on Motzer risk classification. Under Motzer risk classification, RCC patients can have poor, intermediate, or favorable prognoses. In another example, one class of RCC patients has poor prognosis, and the other class of RCC patients has intermediate prognosis. The prognosis gene(s) can be selected from, for instance, Table 11.

In yet another embodiment, the first class of patients has progressive disease after a specified time of treatment, and the second class of patients has non-progressive disease (such as complete response, partial response, minor response, or stable disease) after the same specified time of treatment.

In still yet another embodiment, patients having the solid tumor can be clustered into at least two classes based on their gene expression profiles in PBMCs. Suitable algorithms for this purpose include, but are not limited to, unsupervised clustering analyses. Each of the two classes can be associated with a different respective clinical outcome. For instance, the majority of one class of patients can have a specified TTD (e.g., TTD of less than 365 days), while the majority of the other class of patients can have another specified TTD (e.g., TTD of no less than 365 days). Genes that are substantially correlated with the class distinction between these two classes can be identified. These genes, or the class predictors derived therefrom, can be used to predict the class membership of a patient of interest. In one example, the solid tumor is RCC, and the therapeutic treatment is a CCI-779 therapy. The prognosis gene(s) can be selected from, for instance, Table 16.

Prognosis genes or class predictors that are capable of distinguishing three or more different outcome classes can also be employed in the present invention. These prognosis genes can be identified using multi-class correlation metrics. Suitable programs for carrying out multi-class correlation analysis include, but are not limited to, GeneCluster 2 software (MIT Center for Genome Research at Whitehead Institute, Cambridge, Mass.). Under the analysis, patients having the solid tumor can be divided into at least three classes, and each class has a different respective clinical outcome. The prognosis genes identified under multi-class correlation analysis are differentially expressed in PBMCs of one class of patients relative to PBMCs of other classes of patients. In one embodiment, the identified prognosis genes are substantially correlated with a class distinction between the multiple classes. For instance, the prognosis genes can be selected from those above the 1%, 5%, 10%, 25%, or 50% significance level under a permutation test.

In accordance with another aspect of the present invention, the expression profile of the prognosis gene(s) used in the comparison is correlated with clinical outcome of reference patients under a statistical method. Suitable statistical methods for this purpose include, but are not limited to, Spearman's rank correlation, Cox proportional hazard regression model, or other rank tests or survival models. The reference patients have the same solid tumor as the patient of interest, and the clinical outcome of the reference patients is either known or determinable.

By comparing the expression profile of the prognosis gene(s) in a peripheral blood sample of the patient of interest to the reference expression profile of the same prognosis gene(s) in the reference patients, clinical outcome of the patient of interest can be predicted. For instance, if the expression profile of the patient of interest is more similar to the expression profile of one particular reference patient as compared to other reference patients, clinical outcome of that particular reference patient can be indicative of clinical outcome of the patient of interest.

Any number of prognosis genes can be used for outcome prediction based on statistical methods. In one embodiment, one prognosis gene is used. The reference patient whose expression profile is most similar to that of the patient of interest can be identified. A prediction that clinical outcome of the patient of interest is most analogous to that of the reference patient can therefore be made.

In another embodiment, two or more prognosis genes are used. The expression profile of the patient of interest and the reference expression profile can be compared by a pattern recognition or comparison algorithm. In one example, the Euclidean distance is used to measure the similarity between two different expression profiles.

Any time-associated clinical outcome indicator can be evaluated based on statistical methods. Examples of time-associated clinical outcomes include, but are not limited to, TTP and TTD.

In one embodiment, outcome prediction is based on Spearman's correlation test. The patient of interest and the reference patients have RCC and are being treated by a CCI-779 therapy. In one example, clinical outcome is measured by TTP, and the prognosis gene(s) is selected from Tables 6a and 6b. In another example, clinical outcome is measured by TTD, and the prognosis gene(s) is selected from Tables 6c and 6d. In yet another example, the relative risk for TTD or TTP can be qualitatively assessed based on the peripheral blood expression level of a prognosis gene in the patient of interest, in conjunction with the correlation coefficient of the prognosis gene.

In another embodiment, outcome prediction is based on Cox proportional hazard regression model. The patient of interest and the reference patients have RCC and are being treated by a CCI-779 therapy. In one example, clinical outcome is measured by TTP, and the prognosis gene(s) is selected from Tables 9a and 9b. In another example, clinical outcome is measured by TTD, and the prognosis gene(s) is selected from Tables 9c and 9d. In yet another example, the relative risk for TTD or TTP can be qualitatively assessed based on the peripheral blood expression level of a prognosis gene in the patient of interest, in light of the hazard ratio of the prognosis gene.

In yet another aspect, the present invention provides electronic systems useful for the prognosis or selection of treatment of RCC and other solid tumors. These systems include input or communication devices for receiving the expression profile of the patient of interest as well as the reference expression profile(s). The reference expression profile(s) can be stored in a database or another medium. In one embodiment, the reference expression profile(s) is readily retrievable or modifiable. The comparison between expression profiles can be conduced electronically, such as through a processor or a computer. The processor or computer can execute one or more programs to compare the expression profile of the patient of interest to the reference expression profile(s). The program(s) can be stored in a memory or downloaded from another source, such as an internet server. In one example, the program(s) includes a k-nearest-neighbors or weighted voting algorithm. In another example, the electronic system is coupled to a nucleic acid array and can receive or process expression data generated by the nucleic acid array.

In still another aspect, the present invention provides kits useful for the prognosis or selection of treatment of solid tumors. In one embodiment, the kits of the present invention include probes/primers for detecting expression patterns of one or more solid tumor prognosis genes. Each prognosis gene is differentially expressed in PBMCs of patients who have different clinical outcomes. In many cases, the probe/primers can hybridize under stringent or nucleic acid array hybridization conditions to the RNA transcripts, or the complements thereof, of the corresponding prognosis genes. Hybridization or amplification agents can be included in the kits.

The kits of the present invention can include any number of probes/primers. In one example, each kit includes at least 2, 3, 4, 5, 6, 7, 8, 9, 10, 15, 20, or more different probes/primers, and each of these different probes/primers can hybridize under stringent conditions or nucleic acid array hybridization conditions to a different respective solid tumor prognosis gene. The solid tumor to be prognosed can be RCC, and the prognosis genes can be selected from Tables 6a, 6b, 6c, 6d, 9a, 9b, 9c, 9d, 10, 11, 12, 13, 16, 20 and 21.

In another embodiment, the kits of the present invention include one or more antibodies capable of binding to the polypeptides encoded by respective solid tumor prognosis genes. The antibodies can be, without limitation, polyclonal, monoclonal, single-chain, or humanized. In one example, the antibodies can bind to the respective polypeptide products with affinities of at least 10⁵ M⁻¹, 10⁶ M⁻¹, 10⁷ M⁻¹, or more. In another example, the kits of the present invention include at least 2, 3, 4, 5, 10, 15, 20, or more different antibodies, and each of these different antibodies is capable of binding to a polypeptide encoded by a different respective RCC prognosis gene. The kits of the present invention can also include immunoassay reagents, such as secondary antibodies, controls, or enzyme substrates.

The probes or antibodies of the present invention can be either labeled or unlabeled. Labeled antibodies can be detectable by spectroscopic, photochemical, biochemical, bioelectronic, immunochemical, electrical, optical, chemical, or other suitable means. Exemplary labeling moieties for an antibody include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

The probes or antibodies of the present invention can be enclosed in a vial, a tube, a bottle, a box, or another holding means. In one example, the probes or antibodies are stably attached to one or more substrate supports. Nucleic acid hybridization or immunoassays can be directly carried out on the substrate support(s). Suitable substrate supports include, but are not limited to, glasses, silica, ceramics, nylons, quartz wafers, gels, metals, papers, beads, tubes, fibers, films, membranes, column matrixes, or microtiter plate wells.

IV. Selection of Treatment of RCC and Other Solid Tumors

The present invention allows for personalized treatment of RCC or other solid tumors. Numerous treatment options or regimes can be analyzed by the present invention. Prognosis genes for each treatment can be determined. The peripheral blood expression profiles of these prognosis genes in a patient of interest can be analyzed to identify treatments that have favorable prognoses for the patient of interest. As used herein, a “favorable” prognosis is a prognosis which is better than the average prognosis for all available treatments of the solid tumor.

Any type of cancer treatment can be evaluated by the present invention. For instance, RCC can be treated by drug therapies. Suitable drugs include cytokines, such as interferon or interleukin 2, and chemotherapy drugs, such as CCI-779, AN-238, vinblastine, floxuridine, 5-fluorouracil, or tamoxifen. AN238 is a cytotoxic agent which has 2-pyrrolinodoxorubicin linked to a somatostatin (SST) carrier octapeptide. AN238 can be targeted to SST receptors on the surface of RCC tumor cells. Chemotherapy drugs can be used individually or in combination with other drugs, cytokines, or therapies. In addition, monoclonal antibodies, antiangiogenesis drugs, or anti-growth factor drugs can be employed to treat RCC.

RCC treatment can also be surgical. Suitable surgical choices include, but are not limited to, radical nephrectomy, partial nephrectomy, removal of metastases, arterial embolization, laparoscopic nephrectomy, cryoablation, and nephron-sparing surgery. Moreover, radiation, gene therapy, immunotherapy, adoptive immunotherapy, or any other conventional or experimental therapy can be used.

Treatment options for prostate cancer, head/neck cancer, and other solid tumors are known in the art. For instance, prostate cancer treatments include, but are not limited to, radiation therapy, hormonal therapy, and cryotherapy. The present invention contemplates any novel or experimental treatment of solid tumors.

Prognosis genes or class predictors for each treatment of a solid tumor can be identified according to the present invention. Treatments with favorable prognoses for a patient of interest can therefore be determined. Treatment selection can be conducted manually or electronically. In one embodiment, a reference expression profile database is established for each treatment and each prognosis gene.

Identification of prognosis gene may be affected by the disease stage of a solid tumor. For instance, prognosis genes can be identified from patients at a particular disease stage. Genes thus identified may be more effective in predicting clinical outcome of a patient of interest who is also at that disease stage.

Disease stages may also affect treatment selection. For instance, for RCC patients in stages I or II, radical or partial nephrectomy is commonly selected. For RCC patients in stage III, radical nephrectomy is among the preferred treatments. For RCC patients in stage IV, cytokine immunotherapy, combined immunotherapy and chemotherapy, or other drug therapies can be employed. Therefore, the disease stage of a patient of interest can be used to assist the gene expression-based selection for a favorable treatment of the patient.

It should be understood that the above-described embodiments and the following examples are given by way of illustration, not limitation. Various changes and modifications within the scope of the present invention will become apparent to those skilled in the art from the present description.

### V. EXAMPLES

### Example 1

### Isolation of RNA and Preparation of Labeled Microarray Targets

Prior to initiation of therapy, whole blood samples (8 mL) were collected into Vacutainer sodium citrate cell purification tubes (CPTs) and PBMCs were isolated according to the manufacturer's protocol (Becton Dickinson). All blood samples were shipped in CPTs overnight prior to PBMC processing. PBMCs were purified over Ficoll gradients, washed two times with PBS and counted. Total RNA was isolated from PBMC pellets using the RNeasy mini kit (Qiagen, Valencia, Calif.). Labeled target for oligonucleotide arrays was prepared using a modification of the procedure described in Lockhart, et al., NATURE BIOTECHNOLOGY, 14:1675-80 (1996). 2 μg total RNA was converted to cDNA by priming with an oligo-dT primer containing a T7 DNA polymerase promoter at the 5′ end. The cDNA was used as the template for in vitro transcription using a T7 DNA polymerase kit (Ambion, Woodlands, Tex.) and biotinylated CTP and UTP (Enzo). Labeled cRNA was fragmented in 40 mM Tris-acetate pH 8.0, 100 mM KOAc, 30 mM MgOAc for 35 minutes at 94° C. in a final volume of 40 μl.

### Example 2

### Hybridization to Affymetrix Microarrays and Detection of Fluorescence

Individual RCC samples were hybridized to HgU95A genechip (Affymetrix). No samples were pooled. As described above, 45 RCC patients were involved in the study. Tumors of the RCC patients were histopathologically classified as specific renal cell carcinoma subtypes using the Heidelberg classification of renal cell tumors described in Kovacs, et al., J. PATHOL., 183:131-133 (1997).

10 μg of labeled target was diluted in lx MES buffer with 100 μg/ml herring sperm DNA and 50 μg/ml acetylated BSA. To normalize arrays to each other and to estimate the sensitivity of the oligonucleotide arrays, in vitro synthesized transcripts of 11 bacterial genes were included in each hybridization reaction as described in Hill, et al., SCIENCE, 290: 809-812 (2000). The abundance of these transcripts ranged from 1:300,000 (3 ppm) to 1:1000 (1000 ppm) stated in terms of the number of control transcripts per total transcripts. As determined by the signal response from these control transcripts, the sensitivity of detection of the arrays ranged between about 1:300,000 and 1:100,000 copies/million. Labeled probes were denatured at 99° C. for 5 minutes and then 45° C. for 5 minutes and hybridized to oligonucleotide arrays comprised of over 12,500 human genes (HgU95A, Affymetrix). Arrays were hybridized for 16 hours at 45° C. The hybridization buffer was comprised of 100 mM MES, 1 M [Na⁺], 20 mM EDTA, and 0.01% Tween 20. After hybridization, the cartridges were washed extensively with wash buffer (6×SSPET), for instance, three 10-minute washes at room temperature. These hybridization and washing conditions are collectively referred to as “nucleic acid array hybridization conditions.” The washed cartridges were then stained with phycoerythrin coupled to streptavidin.

12×MES stock contains 1.22 M MES and 0.89 M [Na⁺]. For 1000 ml, the stock can be prepared by mixing 70.4 g MES free acid monohydrate, 193.3 g MES sodium salt and 800 ml of molecular biology grade water, and adjusting volume to 1000 ml. The pH should be between 6.5 and 6.7. 2× hybridization buffer can be prepared by mixing 8.3 ml of 12×MES stock, 17.7 mL of 5 M NaCl, 4.0 mL of 0.5 M EDTA, 0.1 mL of 10% Tween 20 and 19.9 mL of water. 6×SSPET contains 0.9 M NaCl, 60 mM NaH₂PO₄, 6 mM EDTA, pH 7.4, and 0.005% Triton X-100. In some cases, the wash buffer can be replaced with a more stringent wash buffer. 1000 ml stringent wash buffer can be prepared by mixing 83.3 mL of 12×MES stock, 5.2 mL of 5 M NaCl, 1.0 mL of 10% Tween 20 and 910.5 mL of water.

### Example 3

### Gene Expression Data Analysis

Data analysis and absent/present call determination were performed on raw fluorescent intensity values using GENECHIP 3.2 software (Affymetrix). GENECHIP 3.2 software uses algorithms to calculate the likelihood as to whether a gene is “absent” or “present” as well as a specific hybridization intensity value or “average difference” for each transcript represented on the array. For instance, “present” calls are calculated by estimating whether a transcript is detected in a sample based on the strength of the gene's signal compared to background. The algorithms used in these calculations are described in the Affymetrix GeneChip Analysis Suite User Guide (Affymetrix). The “average difference” for each transcript was normalized to “frequency” values according to the procedures of Hill, et al., SCIENCE, 290: 809-812 (2000). This was accomplished by referring the average difference values on each chip to a global calibration curve constructed from the average difference values for the 11 control transcripts with known abundance that were spiked into each hybridization solution. This calibration was used to convert average difference values for all transcripts to frequency estimates, stated in units of parts per million (ppm) ranging from about 1:300,000 (3 ppm) to 1:1000 (1000 ppm). This process also served to normalize between arrays.

Specific transcripts were evaluated further if they met the following criteria. First, genes that were designated “absent” by the GENECHIP 3.2 software in all samples were excluded from the analysis. Second, in comparisons of transcript levels between arrays, a gene was required to be present in at least one of the arrays. Third, for comparisons of transcript levels between groups, a Student's t-test was applied to identify a subset of transcripts that had a significant (p<0.05) differences in frequency values. In certain cases, a fourth criterion, which requires that average fold changes in frequency values across the statistically significant subset of genes be 2-fold or greater, was also used.

Unsupervised hierarchical clustering of genes was performed using the procedure described in Eisen, et al., supra. Nearest-neighbor prediction analysis and supervised cluster analysis was performed using metrics illustrated in Golub, et al., supra. For hierarchical clustering and nearest-neighbor prediction analysis, data were log transformed and normalized to have a mean value of zero and a variance of one. A Student's t-test was used to compare PBMC expression profiles in different outcome classes. In the comparisons, a p value<0.05 can be used to indicate statistical significance.

A k-nearest-neighbor's approach was used to perform a neighborhood analysis of real and randomly permuted data using a correlation metric P(g,c)=(μ1−μ2)/(σ1+σ2), where g is the expression vector of a gene, c is the class vector, μ1 and σ1 define the mean expression level and standard deviation of the gene in class 1, and μ2 and σ2 define the mean expression level and standard deviation of the gene in class 2.

### Example 4

### Gene Expression Analyses Using A More Stringent Filter

In this example, only those transcripts meeting a more stringent data reduction filter were used (at least 25% present calls, and an average frequency across all 45 RCC PBMCs≧5 ppm). This more stringent filter was used to avoid the inclusion low level transcripts in the predictive models. For nearest-neighbor analysis all expression data in training sets and test sets were log transformed prior to analysis. In training sets of data, models containing increasing numbers of features (transcript sequences) were built using a two-sided approach (equal numbers of features in each class) with a S2N similarity metric that used median values for the class estimate. All comparisons were binary distinctions, and each model (with increasing numbers of features) was evaluated by leave one out cross validation. Prediction of class membership in the test sets was performed using a k-nearest-neighbor algorithm in Genecluster version 2.0. In these predictions, the number of neighbors was set to k=3, the cosine distance measure used, and all k neighbors were given equal weights.

As demonstrated above, the Cox proportional hazards regression suggested an association between gene expression and time until disease progression, and an even stronger association between gene expression and survival. On the basis of these findings, a nearest-neighbors algorithm coupled with the stringent data reduction filter was employed to identify multivariate expression patterns in PBMCs that were correlated with and could be used to predict patient outcome. In these analyses, pretreatment expression patterns correlated with the clinical outcomes of TTP and TTD were determined.

In order to evaluate the predictive utility of the profiles correlated with clinical outcomes, 70% of the patient PBMC profiles were randomly selected as a training set, and the remaining 30% of the samples formed the test set. In each approach, the profiles were stratified as originating from patients with poor or favorable outcomes. A nearest-neighbors algorithm was used to generate gene classifiers correlated with groups in the training set. The gene classifier that gave the highest accuracy of class assignment by leave-one-out cross validation was identified. Finally, this gene classifier was evaluated on the test set of samples.

Prior to running these analyses we examined the distribution of PBMC cell types in the various groups to ensure that differences in cell populations were not the sole basis for any observed differences in expression. Tables 18 and 19 demonstrate the distributions of the various cell subtypes (neutrophils, eosinophils, lymphocytes and monocytes) between PBMCs of patients assigned to either good or poor outcome categories for TTP and survival. The mean percentages and the p-value for a t-test (unequal variance) between the good and poor outcome PBMC profiles for each cell subtype are presented. None of the cell subtypes were found to be significantly confounded with the class distinctions for either clinical outcome, ensuring that transcriptional patterns, if identified, would not simply be reflections of altered cell populations between the groups but rather distinct expression patterns arising from PBMC samples with similar cellular compositions.

TABLE 18
Distributions of PBMC Cell Subtypes Between PBMC Profiles
of Patients in Good and Poor Outcome Stratifications
of TTP in Training Set

| Cell Type | TTP > 106 days | TTP < 106 days | p-value |
|---|---|---|---|
| Neutrophil (%) | 24.7 | 30.8 | 0.6885 |
| Eosinophil (%) | 1.6 | 0.7 | 0.1286 |
| Lymphocyte (%) | 47.1 | 37.9 | 0.5789 |
| Monocyte (%) | 26.5 | 30.6 | 0.68 |

TABLE 19
Distributions of PBMC Cell Subtypes Between PBMC Profiles
of Patients in Good and Poor Outcome Stratifications
of TTD in Training Set

| Cell Type | TTD > 365 days | TTP < 365 days | p-value |
|---|---|---|---|
| Neutrophil (%) | 24.3 | 28.8 | 0.7661 |
| Eosinophil (%) | 1.8 | 0.9 | 0.1931 |
| Lymphocyte (%) | 48.5 | 40.5 | 0.5007 |
| Monocyte (%) | 25.4 | 29.8 | 0.5823 |

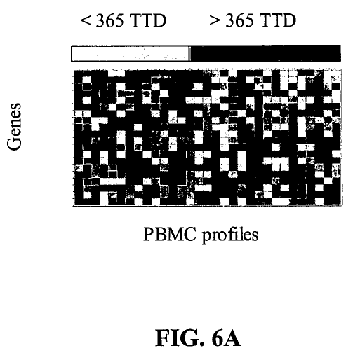

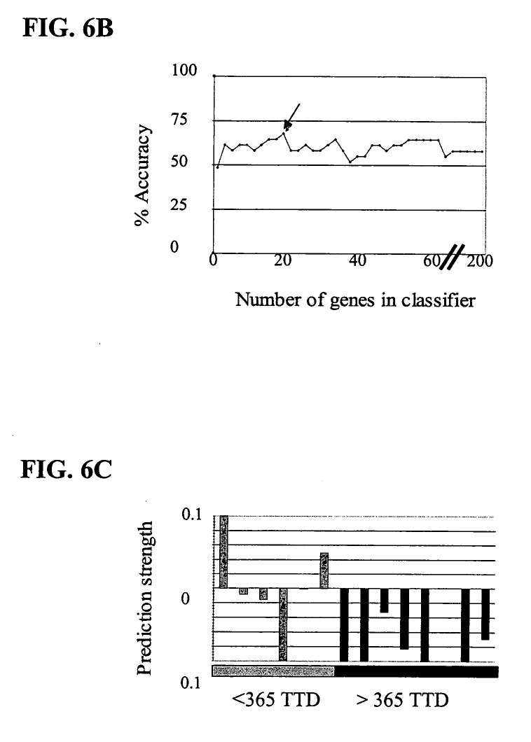

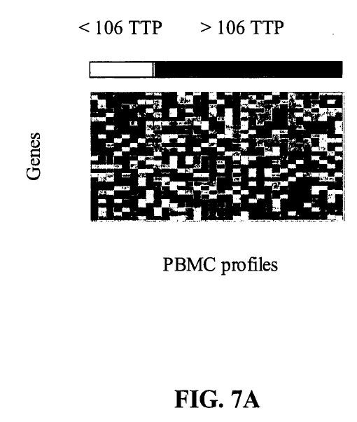

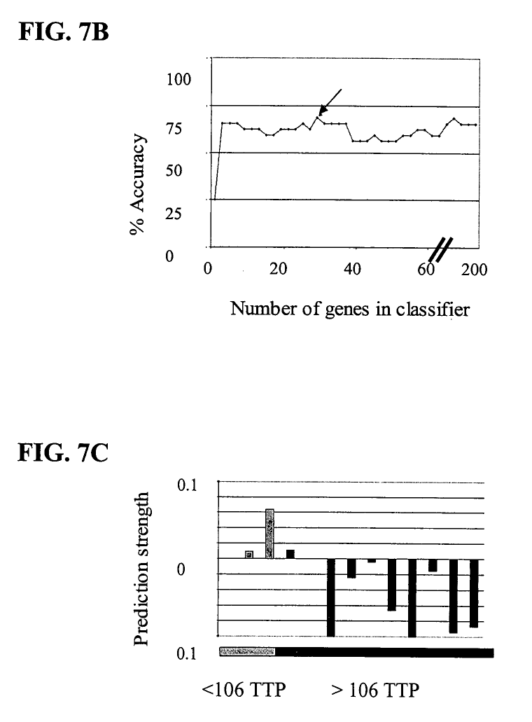

The first analysis is summarized for the comparison of short- and long-term survivors (less than or greater than one year survival) in FIGS. 6A, 6B, and 6C. Patients were stratified as described above into two groups based upon TTD less than or greater than 365 days. A GeneCluster analysis using the signal-to-noise metric identified transcripts correlated with these groups of patients (FIG. 6A). Predictive gene classifiers containing between 2 and 60 genes in steps of 2 (and 60-200 genes in steps of 10) were evaluated by leave-one-out cross validation to identify the smallest predictive model yielding the most accurate class assignments of short- and long-term survivors in the training set. In this comparison the best model found (with respect to leave-one-out cross validation accuracy) was a classifier of 20 genes (FIG. 6B and Table 20). This predictive model was then evaluated using a nearest-neighbors approach on the remaining test set of samples (FIG. 6C). This entire approach was repeated for the stratification of short vs long-term TTP as illustrated in FIGS. 7A, 7B, and 7C. In this comparison the best model found (with respect to leave-one-out cross validation accuracy) was a classifier of 30 genes (FIG. 7B and Table 21), and this predictive model was also evaluated using a nearest-neighbors approach on the remaining test set of samples (FIG. 7C). Further detail concerning overall prediction accuracies, sensitivities and specificities of the predictive models based on year-long survival and time to progression are summarized for the test sets of samples in Table 22.

TABLE 20
Prognosis Genes for Short-term (<365 days) versus Long-term (>365 days) TTD

| SEQ ID NO: | Qualifier | Gene Name | Class | Score | Perm 1% | Perm 5% | Perm (user) |
|---|---|---|---|---|---|---|---|
| 5262 | 33956_at | MD-2 | Less_365_TTD | 0.63 | 1.1363704 | 0.9071798 | 0.66693866 |
| 5247 | 41551_at | RER1 | Less_365_TTD | 0.61 | 1.0375708 | 0.79028875 | 0.6129954 |
| 5022 | 37009_at | UNK_AL035079 | Less_365_TTD | 0.59 | 0.9283793 | 0.77387965 | 0.5757412 |
| 3848 | 35300_at | EPRS | Less_365_TTD | 0.58 | 0.92103595 | 0.74762696 | 0.5645757 |
| 5008 | 39127_f_at | PPP2R4 | Less_365_TTD | 0.56 | 0.8624204 | 0.70808446 | 0.5475367 |
| 5161 | 39360_at | SNX3 | Less_365_TTD | 0.54 | 0.80717504 | 0.6861655 | 0.53616226 |
| 5054 | 41332_at | POLR2E | Less_365_TTD | 0.53 | 0.77077115 | 0.67412776 | 0.52794206 |
| 5092 | 38453_at | ICAM2 | Less_365_TTD | 0.51 | 0.744897 | 0.6632934 | 0.52192914 |
| 4914 | 33424_at | RPN1 | Less_365_TTD | 0.5 | 0.7365122 | 0.64835453 | 0.51936203 |
| | 956_at | TUBB | Less_365_TTD | 0.5 | 0.7222108 | 0.64653593 | 0.51475555 |
| 1045 | 32372_at | CTSB | Greater_365_TTD | 0.82 | 1.2004976 | 0.9564477 | 0.69520277 |
| 692 | 32635_at | KIAA1113 | Greater_365_TTD | 0.81 | 1.0586497 | 0.90758944 | 0.63466245 |
| 1793 | 33493_at | HFL-EDDG1 | Greater_365_TTD | 0.77 | 0.90262204 | 0.8435416 | 0.60823596 |
| 231 | 36474_at | KIAA0776 | Greater_365_TTD | 0.76 | 0.8723624 | 0.78129286 | 0.5796107 |
| 1199 | 31864_at | MPHOSPH6 | Greater_365_TTD | 0.75 | 0.84502566 | 0.7641664 | 0.56468636 |
| 639 | 38317_at | TCEAL1 | Greater_365_TTD | 0.73 | 0.8426697 | 0.7597285 | 0.5504346 |
| 662 | 2064_g_at | ERCC5 | Greater_365_TTD | 0.72 | 0.8337271 | 0.7298645 | 0.5382294 |
| 1154 | 39557_at | UNK_AI625844 | Greater_365_TTD | 0.72 | 0.83215594 | 0.699147 | 0.53125846 |
| 1502 | 36190_at | CDR2 | Greater_365_TTD | 0.71 | 0.8173296 | 0.6975797 | 0.5216159 |
| 151 | 40308_at | UNK_AI830496 | Greater_365_TTD | 0.71 | 0.80752265 | 0.6942027 | 0.51970375 |

TABLE 21
Prognosis Genes for Short-term (<106 days) versus Long-term (>106 days) TTP

| | | | | | | | Perm |
|---|---|---|---|---|---|---|---|
| SEQ ID NO: | Qualifier | Gene Name | Class | Score | Perm 1% | Perm 5% | (user) |
| 4664 | 181_g_at | UNK_S82470 | Less_TTP_106 | 3.41 | 5.582922 | 4.8208075 | 3.5752022 |
| 4559 | 34498_at | VNN2 | Less_TTP_106 | 3 | 5.337237 | 4.2469945 | 3.2616036 |
| 4150 | 38585_at | HBG2 | Less_TTP_106 | 2.95 | 4.1692014 | 3.714144 | 3.099498 |
| 3984 | 39833_at | CHRNE | Less_TTP_106 | 2.85 | 4.067239 | 3.6665761 | 2.9885216 |
| 4430 | 35012_at | MNDA | Less_TTP_106 | 2.84 | 4.032049 | 3.5925848 | 2.9256356 |
| 4894 | 34946_at | DORA | Less_TTP_106 | 2.75 | 3.9986155 | 3.5583446 | 2.8342075 |
| 4067 | 1558_g_at | PAK1 | Less_TTP_106 | 2.7 | 3.8789496 | 3.4725833 | 2.7667618 |
| 4807 | 35820_at | GM2A | Less_TTP_106 | 2.7 | 3.8435366 | 3.4385278 | 2.6919303 |
| 5244 | 41136_s_at | APP | Less_TTP_106 | 2.61 | 3.813862 | 3.3433113 | 2.6589744 |
| 4510 | 32776_at | RALB | Less_TTP_106 | 2.57 | 3.713758 | 3.3420131 | 2.603462 |
| 4816 | 34874_at | NTE | Less_TTP_106 | 2.45 | 3.6834376 | 3.3347135 | 2.5644205 |
| 4509 | 34319_at | S100P | Less_TTP_106 | 2.35 | 3.598251 | 3.2589953 | 2.535933 |
| 3860 | 41102_at | T54 | Less_TTP_106 | 2.31 | 3.5312018 | 3.2556353 | 2.4961586 |
| 4960 | 32046_at | PRKCD | Less_TTP_106 | 2.28 | 3.5278873 | 3.241575 | 2.4784653 |
| 4609 | 36960_at | EDR2 | Less_TTP_106 | 2.25 | 3.4799564 | 3.1926253 | 2.4267142 |
| 2645 | 34871_at | UNK_W30677 | Greater_TTP_106 | 3.89 | 6.951508 | 5.112061 | 4.082164 |
| 757 | 38518_at | SCML2 | Greater_TTP_106 | 3.67 | 5.105945 | 4.6043224 | 3.631336 |
| 2290 | 41189_at | TNFRSF12 | Greater_TTP_106 | 3.59 | 5.105614 | 4.2503996 | 3.395199 |
| 808 | 40048_at | UNK_D43951 | Greater_TTP_106 | 3.49 | 4.7581496 | 4.189143 | 3.3146112 |
| 1733 | 40396_at | P2RX5 | Greater_TTP_106 | 3.49 | 4.513983 | 4.0066333 | 3.2069612 |
| 1063 | 35177_at | KIAA0725 | Greater_TTP_106 | 3.38 | 4.4174356 | 3.9872625 | 3.1314178 |
| 1619 | 40584_at | NUP88 | Greater_TTP_106 | 3.24 | 4.3745546 | 3.9209368 | 3.0728083 |
| 1897 | 38340_at | KIAA0655 | Greater_TTP_106 | 3.23 | 4.121891 | 3.8479779 | 3.009764 |
| 1372 | 37416_at | ARHH | Greater_TTP_106 | 3.22 | 4.105443 | 3.834686 | 2.9688578 |
| 690 | 38148_at | CRY1 | Greater_TTP_106 | 3.19 | 4.051371 | 3.776217 | 2.9163232 |
| 1045 | 32372_at | CTSB | Greater_TTP_106 | 3.18 | 4.0035615 | 3.7531464 | 2.8886828 |
| 119 | 36968_s_at | OIP2 | Greater_TTP_106 | 3.12 | 3.9565299 | 3.6980143 | 2.8398302 |
| 223 | 34256_at | SIAT9 | Greater_TTP_106 | 3.11 | 3.8674347 | 3.6664524 | 2.7820752 |
| 1734 | 41767_r_at | KIAA0855 | Greater_TTP_106 | 3.1 | 3.8383002 | 3.629394 | 2.748495 |
| 1253 | 36403_s_at | UNK_AI434146 | Greater_TTP_106 | 2.96 | 3.778308 | 3.569239 | 2.690984 |

TABLE 22
Performance Characteristics of Gene Classifiers from Supervised
Approaches for Samples in the Test Set

| | Accuracy | Pos Predictive Value | Neg Predictive Value |
|---|---|---|---|
| TTP | 11/13 (85%) | 8/10 (80%) | 3/3 (100%) |
| TTD | 10/14 (72%) | 8/8 (100%) | 2/6 (33%) |

We identified expression patterns and individual transcript levels in pretreatment PBMC expression profiles that appear correlated with, and therefore predictive of, the clinical outcomes of time to progression and survival in patients with RCC.

In initial analyses, an unsupervised hierarchical clustering algorithm segregated patients solely on the basis of the similarity in their global expression profiles in PBMCs. We identified significant differences in survival between these molecularly defined subgroups of patients and, as a precautionary step, tested whether technical or demographic factors were confounded with the observed subgroups of patient PBMC profiles in good and poor outcome clusters. Key technical parameters associated with the profiles (measures of RNA quality, gene chip hybridization, etc) were not significantly different between the groups and therefore did not confound the analysis. In addition we ruled out multiple other demographic parameters (sex, age, ethnicity) as sources of the observed stratification in patient PBMC profiles. Finally, we also determined that CCI-779 dose level did not impact the observed stratifications, indicating that profiles predictive of various outcomes were not CCI-779 dose dependent.

The Kaplan-Meier based differences in survival curves for the subsets of patients in the good versus poor gene expression prognosis clusters were more distinct than the differences in survival for those same patients as predicted by their associated risk classifications (FIGS. 4A and 4B). This finding supports the continued exploration of surrogate tissue profiling for identification of gene expression patterns predictive of outcome, since prior to the expression profiling results in PBMCs reported here, the Motzer risk classification was the prognostic index best correlated with outcome in this clinical study.

Multiple supervised approaches also support the hypothesis that transcriptional levels of select genes in PBMC profiles of RCC patients are significantly correlated with disease progression and survival. Both non-parametric (Spearmans correlation, data not shown) and parametric (Cox proportional hazard modeling) univariate analyses identified individual transcripts that were significantly correlated with both disease progression and survival. Multivariate approaches using k-nearest-neighbor gene selection were also performed to identify multivariate predictors correlated with clinical outcomes of progression and survival. Supervised analyses identified gene signatures in PBMCs that were capable of identifying patients with varying accuracy with respect to TTP and survival. The overall accuracy of these predictive models on test sets of patients was 85% and 72%, respectively, and overall accuracies in both training set cross validation and in test set predictions were similar.

The results further imply that the circulating monocytes, T cells and B cells (or activated neutrophils passing through CPT) may serve as a sensitive monitor of the organism's physiological state. As these cells pass through various tissues, their reaction to the microenvironment is captured in a complex transcriptional response measured through profiling. Surprisingly, such patterns appear to not only be diagnostic of disease state (e.g., RCC) but may also reflect differential responses to variations in the clinically same disease state (e.g., advanced RCC with different degrees of aggressiveness). This suggests that the PBMCs, due to their transit through the body, may serve as an accessible surrogate monitor of tissues and systems that are not easily obtained by routine biopsies.

The functional categories of transcripts in PBMCs associated with low or high risk display several interesting trends. First, transcripts elevated in PBMCs of patients with shorter TTP or survival include those involved in cytoskeletal organization/cell motility, associated small GTPases, general pathways of proteasome-dependent catabolism and general pathways of metabolism. In contrast, transcripts elevated in PBMCs of patients with longer TTP or survival included those involved in mRNA transport, mRNA processing/splicing and ribosomal protein subunits.

Similar surrogate tissue analyses can be used to identify transcriptional profiles that are specific to a particular therapy in question (e.g., CCI-779, interferon-alpha (IFN-α), or CCI-779+IFN-α), as well as those that are simply prognostic of disease outcome regardless of therapy.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. A method comprising comparing an expression profile of at least one gene in a peripheral blood sample of a patient to at least one reference expression profile of said at least one gene, wherein the patient has a solid tumor, and each of said at least one gene is differentially expressed in peripheral blood mononuclear cells of a first class of patients as compared to peripheral blood mononuclear cells of a second class of patients, wherein both the first and second classes of patients have the solid tumor, and wherein the first class of patients has a first clinical outcome, and the second class of patients has a second clinical outcome.

2. The method according to claim 1, wherein the first and second clinical outcomes are outcomes of a therapeutic treatment of the solid tumor in the first and second classes of patients.

3. The method according to claim 2, wherein the expression profile and said at least one reference expression profile are baseline expression profiles for the therapeutic treatment.

4. The method according to claim 2, wherein the peripheral blood sample is a whole blood sample.

5. The method according to claim 2, wherein the peripheral blood sample comprises enriched peripheral blood mononuclear cells.

6. The method according to claim 2, wherein the solid tumor is RCC, and the therapeutic treatment comprises a CCI-779 therapy.

7. The method according to claim 6, wherein the first clinical outcome is TTD of less than a first specified period of time starting from initiation of the therapeutic treatment, and the second clinical outcome is TTD of longer than a second specified period of time starting from initiation of the therapeutic treatment.

8. The method according to claim 6, wherein the first clinical outcome is TTP of less than a specified period of time starting from initiation of the therapeutic treatment, and the second clinical outcome is TTP of longer than another specified period of time starting from initiation of the therapeutic treatment.

9. The method according to claim 6, wherein the first clinical outcome is a Motzer risk classification, and the second clinical outcome is another Motzer risk classification.

10. The method according to claim 2, wherein said at least one gene comprises two or more genes, and said at least one reference expression profile includes a first reference expression profile and a second reference expression profile, wherein the first reference expression profile is an average expression profile of said at least one gene in peripheral blood samples of patients selected from the first class, and the second reference expression profile is an average expression profile of said at least one gene in peripheral blood samples of patients selected from the second class, and wherein the expression profile is compared to said at least one reference expression profile by using a k-nearest-neighbors or weighted voting algorithm.

11. The method according to claim 1, wherein said at least one gene substantially correlates with a class distinction between the first class and the second class.

12. The method according to claim 1, comprising selecting a therapy for treating the solid tumor in the patient, wherein the patient has a favorable prognosis for the therapy.

13. A method comprising comparing an expression profile of at least one gene in a peripheral blood sample of a patient to at least one reference expression profile of said at least one gene, wherein the patient has a solid tumor, and each of said at least one gene is differentially expressed in peripheral blood mononuclear cells of a first class of patients as compared to peripheral blood mononuclear cells of a second class of patients, wherein the first and second classes of patients have the solid tumor, and each of the first and second classes is a subcluster formed by an unsupervised clustering analysis of gene expression profiles in peripheral blood mononuclear cells of a population of patients who have the solid tumor, and wherein the majority of the first class of patients has a first clinical outcome, and the majority of the second class of patients has a second clinical outcome.

14. The method according to claim 13, wherein the first and second clinical outcomes are outcomes of a therapeutic treatment of the solid tumor in the first and second classes of patients, and the expression profile and said at least one reference expression profile are baseline expression profiles for the therapeutic treatment.

15. The method according to claim 14, wherein the solid tumor is RCC, and the therapeutic treatment comprises a CCI-779 therapy.

16. The method according to claim 13, comprising selecting a therapy for treating the solid tumor in the patient, wherein the patient has a favorable prognosis for the therapy.

17. A method comprising comparing an expression profile of at least one gene in a peripheral blood sample of a patient to at least one reference expression profile of said at least one gene, wherein the patient has a solid tumor, and expression levels of each of said at least one gene in peripheral blood mononuclear cells of patients who have the solid tumor correlate with clinical outcomes of said patients.

18. The method according to claim 17, wherein the solid tumor is RCC, and said clinical outcomes are measured by patient response to a CCI-779 therapy, and wherein said at least one gene comprises one or more genes selected from Tables 6a, 6b, 6c, 6d, 9a, 9b, 9c, 9d, 10, 11, 12, 13, 16, 20, and 21.

19. A system comprising:
a memory or a storage medium including data that represent an expression profile of at least one gene in a peripheral blood sample of a patient who has a solid tumor;
at least another storage medium including data that represent at least one reference expression profile of said at least one gene;
a program capable of comparing the expression profile to said at least one reference expression profile; and
a processor capable of executing the program, wherein expression levels of said at least one gene in peripheral blood mononuclear cells of patients who have the solid tumor correlate with clinical outcomes of said patients.

20. A nucleic acid or protein array comprising concentrated probes for solid tumor prognosis genes, wherein each of the solid tumor prognosis genes is differentially expressed in peripheral blood mononuclear cells of a first class of patients as compared to peripheral blood mononuclear cells of a second class of patients, wherein both the first and second classes of patients have a solid tumor, and wherein the first class of patients has a first clinical outcome, and the second class of patients has a second clinical outcome.
